(19) United States
(12) Patent Application Publication
(10) Pub. No.: US 2013/0085283 A1
(43) Pub. Date: Apr. 4, 2013

(54) GERANYLGERANYLACETONE DERIVATIVES

(71) Applicants: Hiroaki Serizawa, Menlo Park, CA (US); Ankush B. Argade, Menlo Park, CA (US); Akash Datwani, Menlo Park, CA (US); Natalie Spencer, Menlo Park, CA (US)

(72) Inventors: Hiroaki Serizawa, Menlo Park, CA (US); Ankush B. Argade, Menlo Park, CA (US); Akash Datwani, Menlo Park, CA (US); Natalie Spencer, Menlo Park, CA (US)

(73) Assignee: Coyote Pharmaceuticals, Inc.

(21) Appl. No.: 13/410,211

(22) Filed: Mar. 1, 2012

Related U.S. Application Data
(60) Provisional application No. 61/543,182, filed on Oct. 4, 2011.

(30) Foreign Application Priority Data
Feb. 29, 2012 (US) PCT/US2012/027147

(51) Int. Cl. C07C 49/203; C07C 69/145; C07C 69/74; C07D 309/00; C07C 205/57; C07C 309/66; C07C 309/73; C07C 69/025; C07C 69/75

(52) U.S. Cl. 549/419; 568/417; 560/129; 560/261; 560/124; 560/122; 560/1; 560/22; 558/44; 558/56

(57) ABSTRACT

Provided herein are geranylgeranylacetone derivatives and methods of using them.

### CROSS REFERENCE TO RELATED APPLICATION

The present application claims benefit under 35 U.S.C. 119(a) of PCT Application No. PCT/US2012/027147, filed Feb. 29, 2012, and claims the benefit under 35 U.S.C. §119(e) of U.S. Provisional Application No. 61/543,182 filed Oct. 4, 2011, both of which are incorporated herein by reference in their entirety.

### FIELD OF THE INVENTION

This invention relates to geranylgeranylacetone derivatives, pharmaceutical compositions comprising such derivatives and uses thereof.

### STATE OF THE ART

Geranylgeranylacetone (GGA) has the formula:

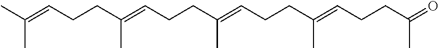 [SMILES: CC(=O)CC/C=C(\C)CC/C=C(\C)CC/C=C(\C)CCC=C(C)C]

and is reported to have neuroprotective and related effects. See, for example, PCT Pat. App. No. PCT/US2011/050071 which is incorporated herein by reference in its entirety.

### SUMMARY OF THE INVENTION

This invention is directed to the discovery of GGA derivatives which also exhibit neuroprotective and related effects. It is contemplated that these derivatives may possess one or more properties such as increased blood brain barrier penetration, enhanced activity, improved serum half-life, and/or lower toxicity.

Accordingly, in one aspect, this invention provides a compound of Formula I:

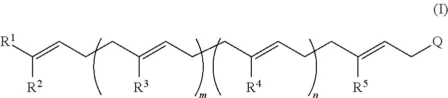

wherein

m is 0 or 1;

n is 0, 1, or 2;

each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups;

each of R³, R⁴, and R⁵ independently are hydrogen or C₁-C₆ alkyl;

Q is selected from the group consisting of:

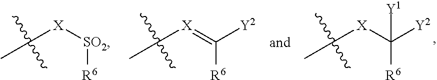

when X is bonded via a single bond, X is —O—, —NR⁷—, or —CR⁸R⁹—, and when X is bonded via a double bond, X is —CR⁸—;

Y¹ is hydrogen or —O—R¹⁰, Y² is —OR¹¹ or —NHR¹², or Y¹ and Y² are joined to form an oxo group (═O), an imine group (═NR¹³), a oxime group (═N—OR¹⁴), or a substituted or unsubstituted vinylidene (═CR¹⁶R¹⁷);

R⁶ is C₁-C₆ alkyl optionally substituted with 1-3 alkoxy or 1-5 halo group, C₂-C₆ alkenyl, C₂-C₆ alkynyl, C₃-C₁₀ cycloalkyl, C₆-C₁₀ aryl, C₃-C₈ heterocyclyl, or C₂-C₁₀ heteroaryl, wherein each cycloalkyl or heterocyclyl is optionally substituted with 1-3 C₁-C₆ alkyl groups, or wherein each aryl or heteroaryl is independently substituted with 1-3 C₁-C₆ alkyl or nitro groups;

R⁷ is hydrogen or together with R⁶ and the intervening atoms form a 5-7 membered ring optionally substituted with 1-3 C₁-C₆ alkyl groups;

each R⁸ and R⁹ independently are hydrogen, C₁-C₆ alkyl, —COR⁸¹ or —CO₂R⁸¹, or R⁸ together with R⁶ and the intervening atoms form a 5-7 membered cycloalkyl or heterocyclyl ring optionally substituted with 1-3 C₁-C₆ alkyl groups;

R¹⁰ is C₁-C₆ alkyl;

R¹¹ and R¹² are independently C₁-C₆ alkyl, C₃-C₁₀ cycloalkyl, —CO₂R¹⁵, or —CON(R¹⁵)₂, or R¹⁰ and R¹¹ together with the intervening carbon atom and oxygen atoms form a 5-6 membered heterocycle optionally substituted with 1-3 C₁-C₆ alkyl groups;

R¹³ is C₁-C₆ alkyl or C₃-C₁₀ cycloalkyl optionally substituted with 1-3 C₁-C₆ alkyl groups;

R¹⁴ is hydrogen, C₁-C₆ alkyl optionally substituted with a —CO₂H or an ester thereof or a C₆-C₁₀ aryl, C₂-C₆ alkenyl, C₂-C₆ alkynyl, C₃-C₁₀ cycloalkyl, or a C₃-C₈ heterocyclyl, wherein each cycloalkyl, heterocyclyl, or aryl, is optionally substituted with 1-3 alkyl groups;

each R¹⁵ independently are hydrogen, C₃-C₁₀ cycloalkyl, C₁-C₆ alkyl optionally substituted with 1-3 substituents selected from the group consisting of —CO₂H or an ester thereof, C₆-C₁₀ aryl, or C₃-C₈ heterocyclyl, or two R¹⁵ groups together with the nitrogen atom they are bonded to form a 5-7 membered heterocycle;

R¹⁶ is hydrogen or C₁-C₆ alkyl;

R¹⁷ is hydrogen, C₁-C₆ alkyl substituted with 1-3 hydroxy groups, —CHO, or is CO₂H or an ester thereof; and

each R⁸¹ independently is C₁-C₆ alkyl; and

provided that the compound excludes the compound of formula:

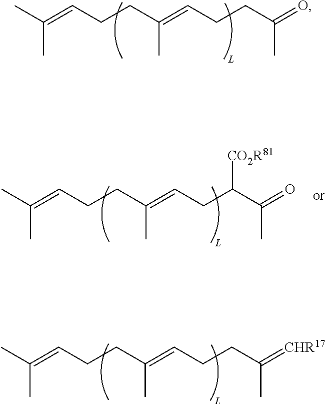

wherein L is 0, 1, 2, or 3, and R¹⁷ is CO₂H or an ester thereof or is —CH₂OH.

In another aspect, this invention provides a composition comprising a GGA derivative provided herein and a pharmaceutically acceptable excipient.

In another aspect, this invention provides a method for treating a neuron in need thereof of one or more of: (i) neuroprotection of the neuron at risk of neural damage or death, (ii) increasing the axon growth of the neuron, (iii) inhibiting the cell death of the neuron susceptible to neuronal cell death, (iv) increasing the neurite growth of the neuron, and/or (v) neurostimulation comprising increasing the expression and/or the release of one or more neurotransmitters from the neuron, the method comprising contacting said neurons with an effective amount of a compound or a composition provided herein.

In one embodiment, a pre-contacted neuron exhibits one or more of: (i) a reduction in the axon growth ability, (ii) a reduced expression level of one or more neurotransmitters, (iii) a reduction in the formation of synapses, and/or (iv) a reduction in electrical excitability. In another embodiment, the neurostimulation further comprises one or more of: (i) enhancing or inducing synapse formation of a neuron, (ii) increasing or enhancing electrical excitability of a neuron, (iii) modulating the activity of G proteins in neurons, and (iv) enhancing the activation of G proteins in neurons.

In another aspect, this invention provides a method for inhibiting the loss of cognitive abilities in a mammal that is at risk of dementia or suffering from incipient or partial dementia while retaining some cognitive skills which method comprises contacting said neuron with an effective amount of a compound or a composition provided herein.

In another aspect, this invention provides a method for inhibiting the death of neurons due to formation of or further formation of pathogenic protein aggregates either between, outside or inside neurons, wherein said method comprises contacting said neurons at risk of developing said pathogenic protein aggregates with a protein aggregate inhibiting amount of a compound or a composition provided herein. In another embodiment. The pathogenic protein aggregates from between, outside, and/or inside said neurons.

In another aspect, this invention provides a method for inhibiting the neurotoxicity of β-amyloid peptide by contacting the β-amyloid peptide with an effective amount of a compound or a composition provided herein. In another embodiment, the β-amyloid peptide is between or outside of neurons, or is part of the β-amyloid plaque.

In another aspect, this invention provides a method for inhibiting neural death and/or increasing neural activity in a mammal suffering from a neural disease, wherein the etiology of said neural disease comprises formation of protein aggregates which are pathogenic to neurons which method comprises administering to said mammal an amount of a compound or a composition of provided herein, which will inhibit further pathogenic protein aggregation provided that said pathogenic protein aggregation is not intranuclear.

In another aspect, this invention provides a method for inhibiting neural death and/or increasing neural activity in a mammal suffering from ALS or AD, wherein the etiology of said ALS or AD comprises formation of protein aggregates which are pathogenic to neurons which method comprises administering to said mammal an amount of a compound or a composition provided herein, which will inhibit further pathogenic protein aggregation provided that said pathogenic protein aggregation is not related to SBMA. In another embodiment, the amount of the compound provided herein administered alters the pathogenic protein aggregate present into a non-pathogenic form or prevents formation of pathogenic protein aggregates.

In another aspect, this invention provides a method for preventing neural death during seizures in a mammal in need thereof, which method comprises administering a therapeutically effective amount of a compound or a composition provided herein.

In certain preferred embodiments, the therapeutically effective amount of the compound is 1-12 mg/kg. In certain more preferred embodiments, the therapeutically effective amount is 1-5 mg/kg or 6-12 mg/kg. In certain still more preferred embodiments, the therapeutically effective amount is 3 mg/kg, 6 mg/kg, or 12 mg/kg.

### DETAILED DESCRIPTION OF THE INVENTION

This invention relates to geranylgeranylacetone derivatives and uses thereof. However, prior to describing this invention in greater detail, the following terms will first be defined.

It must be noted that as used herein and in the appended claims, the singular forms “a”, “an”, and “the” include plural referents unless the context clearly dictates otherwise. Thus, for example, reference to “a solvent” includes a plurality of such solvents.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations. Each numerical parameter should at least be construed in light of the number of reported significant digits and by applying ordinary rounding techniques.

As used herein, Cₘ-Cₙ, such as C₁-C₁₀, C₁-C₆, or C₁-C₄ when used before a group refers to that group containing m to n carbon atoms.

The term “about” when used before a numerical designation, e.g., temperature, time, amount, and concentration, including range, indicates approximations which may vary by (+) or (−) 10%, 5% or 1%.

As used herein, the term “AD” refers to Alzheimer's disease.

The term “alkyl” refers to monovalent saturated aliphatic hydrocarbyl groups having from 1 to 10 carbon atoms (i.e., C₁-C₁₀ alkyl) or 1 to 6 carbon atoms (i.e., C₁-C₆ alkyl), or 1 to 4 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). Alkyl substituted with a substituent refers to an alkyl group that is substituted with up to 5, preferably up to 4, and still more preferably up to 3 substituents, and includes alkyl groups substituted with 1 or 2 substituents.

The term “alkenyl” refers to monovalent aliphatic hydrocarbyl groups having from 2 to 10 carbon atoms or 2 to 6 carbon atoms and 1 or more, preferably 1, carbon carbon double bond. Examples of alkenyl include vinyl, allyl, dimethyl allyl, and the like.

The term “alkoxy” refers to —O-alkyl, where alkyl is as defined above.

The term “alkynyl” refers to monovalent aliphatic hydrocarbyl groups having from 2 to 10 carbon atoms or 2 to 6 carbon atoms and 1 or more, preferably 1, carbon carbon triple bond —(C≡C)—. Examples of alkenyl include ethynyl, propargyl, dimethylpropargyl, and the like.

The term “aryl” refers to a monovalent, aromatic mono- or bicyclic ring having 6-10 ring carbon atoms. Examples of aryl include phenyl and naphthyl. The condensed ring may or may not be aromatic provided that the point of attachment is at an aromatic carbon atom. For example, and without limitation, the following is an aryl group:

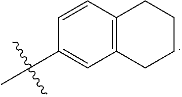 [SMILES: *c1ccc2c(c1)CCCC2]

As used herein, the term “ALS” refers to amyotrophic lateral sclerosis disease.

The term “axon” refers to projections of neurons that conduct signals to other cells through synapses. The term “axon growth” refers to the extension of the axon projection via the growth cone at the tip of the axon.

The term “—CO₂H ester” refers to an ester formed between the —CO₂H group and an alcohol, preferably an aliphatic alcohol. A preferred example included —CO₂R^{E}, wherein R^{E} is alkyl or aryl group.

The term “cycloalkyl” refers to a monovalent, preferably saturated, hydrocarbyl mono-, bi-, or tricyclic ring having 3-12 ring carbon atoms. While cycloalkyl, refers preferably to saturated hydrocarbyl rings, as used herein, it also includes rings containing 1-2 carbon-carbon double bonds. Nonlimiting examples of cycloalkyl include cyclopropyl, cyclobutyl, cyclopentyl, cyclohexyl, cycloheptyl, adamentyl, and the like. The condensed rings may or may not be non-aromatic hydrocarbyl rings provided that the point of attachment is at a cycloalkyl carbon atom. For example, and without limitation, the following is a cycloalkyl group:

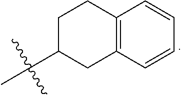 [SMILES: *C1CCc2ccccc2C1]

The term “cytoplasm” refers to the space outside of the nucleus but within the outer cell wall of an animal cell.

The term “G protein” refers to a family of proteins involved in transmitting chemical signals outside the cell and causing changes inside of the cell. The Rho family of G proteins is small G protein, which are involved in regulating actin cytoskeletal dynamics, cell movement, motility, transcription, cell survival, and cell growth. RHOA, RAC1, and CDC42 are the most studied proteins of the Rho family. Active G proteins are localized to the cellular membrane where they exert their maximal biological effectiveness.

The term “halo” refers to F, Cl, Br, and I.

The term “heteroaryl” refers to a monovalent, aromatic mono-, bi-, or tricyclic ring having 2-14 ring carbon atoms and 1-6 ring heteroatoms selected preferably from N, O, S, and P and oxidized forms of N, S, and P, provided that the ring contains at least 5 ring atoms. Nonlimiting examples of heteroaryl include furan, imidazole, pyridine, quinoline, and the like. The condensed rings may or may not be a heteroatom containing aromatic ring provided that the point of attachment is a heteroaryl atom. For example, and without limitation, the following is a heteroaryl group:

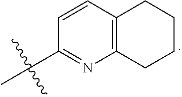 [SMILES: *c1ccc2c(n1)CCCC2]

The term “heterocyclyl” or heterocycle refers to a non-aromatic, mono-, bi-, or tricyclic ring containing 2-10 ring carbon atoms and 1-6 ring heteroatoms selected preferably from N, O, S, and P and oxidized forms of N, S, and P, provided that the ring contains at least 3 ring atoms. While heterocyclyl preferably refers to saturated ring systems, it also includes ring systems containing 1-3 double bonds, provided that they ring is non-aromatic. Nonlimiting examples of heterocyclyl include, piperidinyl, piperazinyl, pyrrolidinyl, tetrahydrofuranyl, and tetrahydropyranyl. The condensed rings may or may not contain a non-aromatic heteroatom containing ring provided that the point of attachment is a heterocyclyl group. For example, and without limitation, the following is a heterocyclyl group:

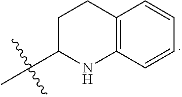 [SMILES: *C1CCc2ccccc2N1]

The term “intranuclear” or “intranuclearly” refers to the space inside the nuclear compartment of an animal cell.

The term “neural disease” refers to diseases that compromise the cell viability of neurons. Neural diseases in which the etiology of said neural disease comprises formation of protein aggregates which are pathogenic to neurons provided that the protein aggregates are not related to the disease SBMA and are not intranuclear, include but are not limited to ALS, AD, Parkinson's Disease, multiple sclerosis, and prion diseases such as Kuru, Creutzfeltdt-Jakob disease, Fatal familial insomnia, and Gerstmann-Straussler-Scheinker syndrome. These neural diseases are also different from SBMA in that they do not contain polyglutamine repeats. Neural diseases can be recapitulated in vitro in tissue culture cells. For example, AD can be modeled in vitro by adding pre-aggregated β-amyloid peptide to the cells. ALS can be modeled by depleting an ALS disease-related protein, TDP-43. Neural disease can also be modeled in vitro by creating protein aggregates through providing toxic stress to the cell. One way this can be achieved is by mixing dopamine with neurons such as neuroblastoma cells. These neural diseases can also be recapitulated in vivo in mouse models. A transgenic mouse that expresses a mutant Sod1 protein has similar pathology to humans with ALS. Similarly, a transgenic mouse that overexpresses APP has similar pathology to humans with AD.

The term “neuron” or “neurons” refers to all electrically excitable cells that make up the central and peripheral nervous system. The neurons may be cells within the body of an animal or cells cultured outside the body of an animal. The term “neuron” or “neurons” also refers to established or primary tissue culture cell lines that are derived from neural cells from a mammal or tissue culture cell lines that are made to differentiate into neurons. “Neuron” or “neurons” also refers to any of the above types of cells that have also been modified to express a particular protein either extrachromosomally or intrachromosomally. “Neuron” or “neurons” also refers to transformed neurons such as neuroblastoma cells and support cells within the brain such as glia.

The term “neuroprotective” refers to reduced toxicity of neurons as measured in vitro in assays where neurons susceptible to degradation are protected against degradation as compared to control. Neuroprotective effects may also be evaluated in vivo by counting neurons in histology sections.

The term “neurotransmitter” refers to chemicals which transmit signals from a neuron to a target cell. Examples of neurotransmitters include but are not limited to: amino acids such as glutamate, aspartate, serine, γ-aminobutyric acid, and glycine; monoamines such as dopamine, norepinephrine, epinephrine, histamine, serotonin, and melatonin; and other molecules such as acetycholine, adenosine, anadamide, and nitric oxide.

The term “protein aggregates” refers to a collection of proteins that may be partially or entirely mis-folded. The protein aggregates may be soluble or insoluble and may be inside the cell or outside the cell in the space between cells. Protein aggregates inside the cell can be intranuclear in which they are inside the nucleus or cytoplasm in which they are in the space outside of the nucleus but still within the cell membrane. The protein aggregates described in this invention are granular protein aggregates.

The term “protein aggregate inhibiting amount” refers to an amount of GGA that inhibits the formation of protein aggregates at least partially or entirely. Unless specified, the inhibition could be directed to protein aggregates inside the cell or outside the cell.

The term “pathogenic protein aggregate” refers to protein aggregates that are associated with disease conditions. These disease conditions include but are not limited to the death of a cell or the partial or complete loss of the neuronal signaling among two or more cells. Pathogenic protein aggregates can be located inside of a cell, for example, pathogenic intracellular protein aggregates or outside of a cell, for example, pathogenic extracellular protein aggregates.

As used herein, the term “SBMA” refers to the disease spinal and bulbar muscular atrophy. Spinal and bulbar muscular atrophy is a disease caused by pathogenic androgen receptor protein accumulation intranuclearly.

The term “synapse” refers to junctions between neurons. These junctions allow for the passage of chemical signals from one cell to another.

As used herein, the term “treatment” or “treating” means any treatment of a neuron or a disease or condition related to neurons in a patient, ex vivo, or in vitro, including one or more of: preventing or protecting against the disease or condition, that is, causing the relevant symptoms not to develop, for example, in a subject or a neuron at risk of suffering from such a disease or condition, thereby substantially averting onset of the disease or condition; inhibiting the disease or condition, that is, arresting or suppressing the development of relevant symptoms; and relieving the disease or condition that is, causing the regression of relevant symptoms.

### Geranylgeranylacetone Derivatives

In one aspect, this invention provides a compound of Formula I:

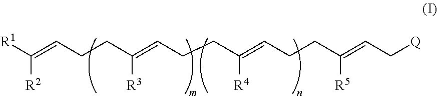

wherein

m is 0 or 1;

n is 0, 1, or 2;

each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups;

each of R³, R⁴, and R⁵ independently are hydrogen or C₁-C₆ alkyl;

Q is selected from the group consisting of:

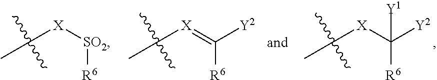

when X is bonded via a single bond, X is —O—, —NR⁷—, or —CR⁸R⁹—, and when X is bonded via a double bond, X is —CR⁸—;

Y¹ is hydrogen or —O—R¹⁰, Y² is —OR¹¹ or —NHR¹², or Y¹ and Y² are joined to form an oxo group (═O), an imine group (═NR¹³), a oxime group (═N—OR¹⁴), or a substituted or unsubstituted vinylidene (═CR¹⁶R¹⁷);

R⁶ is C₁-C₆ alkyl optionally substituted with 1-3 alkoxy or 1-5 halo group, C₂-C₆ alkenyl, C₂-C₆ alkynyl, C₃-C₁₀ cycloalkyl, C₆-C₁₀ aryl, C₃-C₈ heterocyclyl, or C₂-C₁₀ heteroaryl, wherein each cycloalkyl or heterocyclyl is optionally substituted with 1-3 C₁-C₆ alkyl groups, or wherein each aryl or heteroaryl is independently substituted with 1-3 C₁-C₆ alkyl or nitro groups;

R⁷ is hydrogen or together with R⁶ and the intervening atoms form a 5-7 membered ring optionally substituted with 1-3 C₁-C₆ alkyl groups;

each R⁸ and R⁹ independently are hydrogen, C₁-C₆ alkyl, —COR⁸¹ or —CO₂R⁸¹, or R⁸ together with R⁶ and the intervening atoms form a 5-7 membered cycloalkyl or heterocyclyl ring optionally substituted with 1-3 C₁-C₆ alkyl groups;

R¹⁰ is C₁-C₆ alkyl;

R¹¹ and R¹² are independently C₁-C₆ alkyl, C₃-C₁₀ cycloalkyl, —CO₂R¹⁵, or —CON(R¹⁵)₂, or R¹⁰ and R¹¹ together with the intervening carbon atom and oxygen atoms form a 5-6 membered heterocycle optionally substituted with 1-3 C₁-C₆ alkyl groups;

R¹³ is C₁-C₆ alkyl or C₃-C₁₀ cycloalkyl optionally substituted with 1-3 C₁-C₆ alkyl groups;

R¹⁴ is hydrogen, C₁-C₆ alkyl optionally substituted with a —CO₂H or an ester thereof or a C₆-C₁₀ aryl, C₂-C₆ alkenyl, C₂-C₆ alkynyl, C₃-C₁₀ cycloalkyl, or a C₃-C₈ heterocyclyl, wherein each cycloalkyl, heterocyclyl, or aryl, is optionally substituted with 1-3 alkyl groups;

each R¹⁵ independently are hydrogen, C₃-C₁₀ cycloalkyl, C₁-C₆ alkyl optionally substituted with 1-3 substituents selected from the group consisting of —CO₂H or an ester thereof, C₆-C₁₀ aryl, or C₃-C₈ heterocyclyl, or two R¹⁵ groups together with the nitrogen atom they are bonded to form a 5-7 membered heterocycle;

R¹⁶ is hydrogen or C₁-C₆ alkyl;

R¹⁷ is hydrogen, C₁-C₆ alkyl substituted with 1-3 hydroxy groups, —CHO, or is CO₂H or an ester thereof; and

each R⁸¹ independently is C₁-C₆ alkyl; and

provided that the compound excludes the compound of formula:

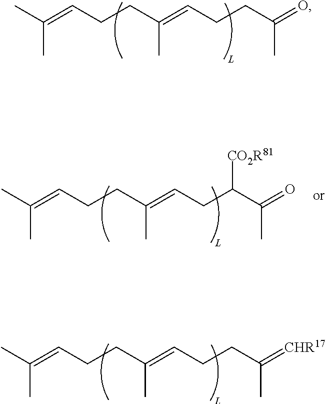

wherein L is 0, 1, 2, or 3, and R¹⁷ is CO₂H or an ester thereof or is —CH₂OH.

In one embodiment, m is 0. In another embodiment, m is 1. In another embodiment, n is 0. In another embodiment, n is 1. In another embodiment, n is 2.

In one embodiment, the compound of Formula (I) is of formula:

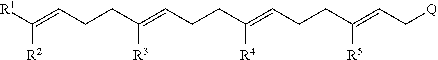

wherein R¹, R², R³, R⁴, R⁵, and Q are defined as in any aspect or embodiment here.

In one embodiment, the compound provided is of formula:

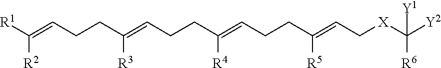

wherein R¹, R², R³, R⁴, R⁵, R⁶, X, Y¹, and Y² are defined as in any aspect and embodiment here.

In another embodiment, the compound provided is of formula:

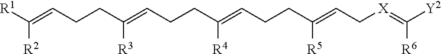

wherein R¹, R², R³, R⁴, R⁵, R⁶, X, and Y² are defined as in any aspect and embodiment here.

In another embodiment, the compound provided is of formula:

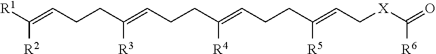

wherein R¹, R², R³, R⁴, R⁵, R⁶ and X are defined as in any aspect and embodiment here.

In another embodiment, the compound provided is of formula:

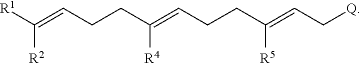

wherein R¹, R², R⁴, R⁵, and Q are defined as in any aspect and embodiment here.

In another embodiment, the compound provided is of formula:

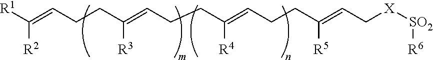

wherein R¹, R², R⁴, R⁵, m, n, X, and R⁶ are defined as in any aspect and embodiment here.

In another embodiment, the compound provided is of formula:

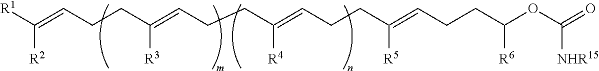

wherein R¹, R², R⁴, R⁵, R⁶, m, n, and R¹⁵ are defined as in any aspect and embodiment here.

In another embodiment, this invention provides a compound of Formula Ia:

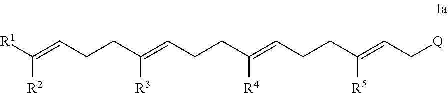

wherein each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups;

each of R³, R⁴, and R⁵ independently are hydrogen or C₁-C₆ alkyl;

Q is selected from the group consisting of:

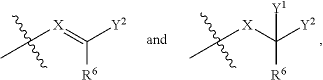

when X is bonded via a single bond, X is —O—, —NR⁷—, or —CR⁸R⁹—, and when X is bonded via a double bond, X is —CR⁸—;

Y¹ is absent or is hydrogen or —O—R¹⁰, Y² is —OR¹¹ or —NHR¹², or Y¹ and Y² are joined to form an oxo group (═O), an imine group (═NR¹³) or a oxime group (═N—OR¹⁴);

R⁶ is C₁-C₆ alkyl, C₁-C₆ alkyl substituted with 1-3 alkoxy or 1-5 halo group, C₂-C₆ alkenyl, C₂-C₆ alkynyl, C₃-C₁₀ cycloalkyl optionally substituted with 1-3 C₁-C₆ alkyl groups, C₆-C₁₀ aryl, or C₂-C₁₀ heteroaryl;

R⁷ is hydrogen or together with R⁶ and the intervening atoms form a 5-7 membered ring optionally substituted with 1-3 C₁-C₆ alkyl groups;

each R⁸ and R⁹ independently are hydrogen, C₁-C₆ alkyl, or —CO₂R⁸¹, or R⁸ together with R⁶ and the intervening atoms form a 5-7 membered ring optionally substituted with 1-3 C₁-C₆ alkyl groups;

R¹⁰ is C₁-C₆ alkyl;

R¹¹ and R¹² are independently C₁-C₆ alkyl, C₃-C₆ cycloalkyl, or —CON(R¹⁵)₂;

R¹³ is C₁-C₆ alkyl or C₃-C₇ cycloalkyl;

R¹⁴ is hydrogen, C₁-C₆ alkyl or C₃-C₆ cycloalkyl;

each R¹⁵ independently are hydrogen or C₁-C₆ alkyl or two R¹⁵ groups together with the nitrogen atom they are bonded to form a 5-7 membered heterocycle; and

R⁸¹ is C₁-C₆ alkyl; and

provided that the compound excludes the compound of formula:

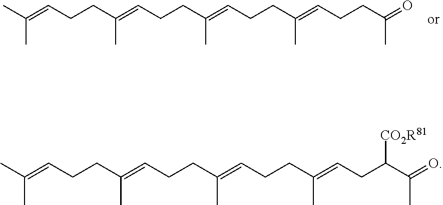

In another embodiment, each R¹ and R² are C₁-C₆ alkyl. In another embodiment, each R¹ and R² are methyl, ethyl, or isopropyl. In another embodiment, R¹ and R² together with the carbon atom they are attached to form a 5-6 membered ring optionally substituted with 1-3 C₁-C₆ alkyl groups. In another embodiment, R¹ and R² together with the carbon atom they are attached to form a ring that is:

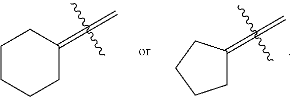 [SMILES: *=C1CCCCC1 ; *=C1CCCC1]

In another embodiment, R³, R⁴, and R⁵ are C₁-C₆ alkyl. In another embodiment, one of R³, R⁴, and R⁵ are alkyl, and the rest are hydrogen. In another embodiment, two of R³, R⁴, and R⁵ are alkyl, and the rest are hydrogen. In another embodiment, R³, R⁴, and R⁵ are hydrogen. In another embodiment, R³, R⁴, and R⁵ are methyl.

In another embodiment, X is O. In another embodiment, X is —NR⁷. In another embodiment, R⁷ is hydrogen. In another embodiment, R⁷ together with R⁶ and the intervening atoms form a 5-7 membered ring optionally substituted with 1-3 C₁-C₆ alkyl groups. In another embodiment, X is —CR⁸R⁹—. In another embodiment, X is —CR⁸—. In another embodiment, each R⁸ and R⁹ independently are hydrogen, C₁-C₆ alkyl, —COR⁸¹, or —CO₂R⁸¹. In another embodiment, R⁸ is hydrogen, and R⁹ is hydrogen, C₁-C₆ alkyl, —COR⁸¹, or —CO₂R⁸¹.

In another embodiment, R⁹ is hydrogen. In another embodiment, R⁹ C₁-C₆ alkyl. In another embodiment, R⁹ is methyl. In another embodiment, R⁹ is —CO₂R⁸¹. In another embodiment, R⁹ is —COR⁸¹.

In another embodiment, R⁸ together with R⁶ and the intervening atoms form a 5-7 membered ring. In another embodiment, the moiety:

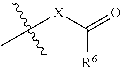

which is “Q,” has the structure:

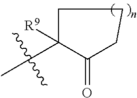

wherein R⁹ is hydrogen, C₁-C₆ alkyl, or —CO₂R⁸¹ and n is 1, 2, or 3. Within these embodiments, in certain embodiments, R⁹ is hydrogen or C₁-C₆ alkyl. In one embodiment, R⁹ is hydrogen. In another embodiment, R⁹ is C₁-C₆ alkyl.

In another embodiment, R⁶ is C₁-C₆ alkyl. In another embodiment, R⁶ is methyl, ethyl, butyl, isopropyl, or tertiary butyl. In another embodiment, R⁶ is C₁-C₆ alkyl substituted with 1-3 alkoxy or 1-5 halo group. In another embodiment, R⁶ is alkyl substituted with an alkoxy group. In another embodiment, R⁶ is alkyl substituted with 1-5, preferably, 1-3, halo, preferably fluoro, groups.

In another embodiment, R⁶ is C₂-C₆ alkenyl or C₂-C₆ alkynyl. In another embodiment, R⁶ is C₃-C₁₀ cycloalkyl. In another embodiment, R⁶ is C₃-C₁₀ cycloalkyl substituted with 1-3 C₁-C₆ alkyl groups. In another embodiment, R⁶ is cyclopropyl, cyclobutyl, cyclopentyl, cyclohexyl, or adamentyl. In another embodiment, R⁶ is C₆-C₁₀ aryl or C₂-C₁₀ heteroaryl. In another embodiment, R⁶ is a 5-7 membered heteroaryl containing at least 1 oxygen atom. In another embodiment, R⁶ is C₆-C₁₀ aryl, C₃-C₈ heterocyclyl, or C₂-C₁₀ heteroaryl, wherein each aryl, heterocyclyl, or heteroaryl is optionally substituted with 1-3 C₁-C₆ alkyl groups.

In another embodiment, Y² is —O—R¹¹. In another embodiment, Y¹ and Y² are joined to form ═NR¹³. In another embodiment, Y¹ and Y² are joined to form ═NOR¹⁴. In another embodiment, Y¹ and Y² are joined to form (═O). In another embodiment, Y¹ and Y² are joined to form ═CR¹⁶R¹⁷.

In another embodiment, Q is —CR⁹COR⁶. In another embodiment, R⁶ is C₁-C₆ alkyl optionally substituted with an alkoxy group. In another embodiment, R⁶ is C₃-C₈ cycloalkyl. In another embodiment, R⁹ is hydrogen. In another embodiment, R⁹ is C₁-C₆ alkyl. In another embodiment, R⁹ is CO₂R⁸¹. In another embodiment, R⁹ is COR⁸¹.

In another embodiment, Q is —CH₂—CH(O—CONHR¹⁵)—R⁶. In another embodiment, R¹⁵ is C₃-C₈ cycloalkyl. In another embodiment, R¹⁵ is C₁-C₆ alkyl optionally substituted with 1-3 substituents selected from the group consisting of —CO₂H or an ester thereof, C₆-C₁ aryl, or C₃-C₈ heterocyclyl. In a preferred embodiment within these embodiments, R⁶ is C₁-C₆ alkyl.

In another embodiment, R¹⁴ is hydrogen. In another embodiment, R¹⁴ is C₁-C₆ alkyl optionally substituted with a —CO₂H or an ester thereof or a C₆-C₁₀ aryl optionally substituted with 1-3 alkyl groups. In another embodiment, R¹⁴ is C₂-C₆ alkenyl. In another embodiment, R¹⁴ is C₂-C₆ alkynyl In another embodiment, R¹⁴ is C₃-C₆ cycloalkyl optionally substituted with 1-3 alkyl groups. In another embodiment, R¹⁴ is C₃-C₈ heterocyclyl optionally substituted with 1-3 alkyl groups.

In another embodiment, preferably, R¹⁶ is hydrogen. In another embodiment, R¹⁷ is CO₂H or an ester thereof. In another embodiment, R¹⁷ is C₁-C₆ alkyl substituted with 1-3 hydroxy groups. In another embodiment, R¹⁷ is C₁-C₃ alkyl substituted with 1 hydroxy group. In another embodiment, R¹⁷ is —CH₂OH.

In another embodiment, R¹⁰ and R¹¹ together with the intervening carbon atom and oxygen atoms form a heteroycle of formula:

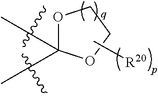

wherein q is 0 or 1, p is 0, 1, 2, or 3, and R²⁰ is C₁-C₆ alkyl.

In another embodiment, q is 1. In another embodiment, q is 2. In another embodiment, p is 0. In another embodiment, p is 1. In another embodiment, p is 2. In another embodiment, p is 3.

In another embodiment, examples of compounds provided by this invention include certain compounds tabulated below and certain compounds described in Example 1 as will be apparent to the skilled artisan upon reading this disclosure. Certain known compounds are included in the table to demonstrate the usefulness of these compounds in the methods provided herein. All the tested compounds showed certain neoroprotective activity:

TABLE 1

| | Activity | | |
|---|---|---|---|
| | | 10 | |
| Chemical Structure | 1 nm | nM | 1 μM |
| 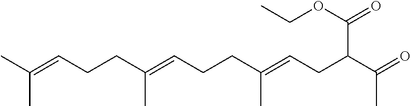 [SMILES: CCOC(=O)C(C/C=C(\C)CC/C=C(\C)CCC=C(C)C)C(C)=O] | | | 259* |
| 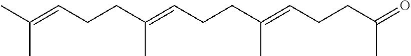 [SMILES: CC(=O)CC/C=C(\C)CC/C=C(\C)CCC=C(C)C] | | 192* | |
| 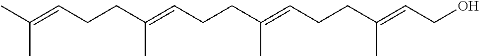 [SMILES: CC(C)=CCC/C(C)=C/CC/C(C)=C/CC/C(C)=C/CO] | | 295* | |
| 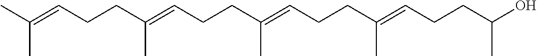 [SMILES: CC(C)=CCC/C(C)=C/CC/C(C)=C/CC/C(C)=C/CCC(C)O] | | 170 | |
| 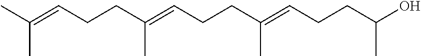 [SMILES: CC(C)=CCC/C(C)=C/CC/C(C)=C/CCC(C)O] | | 224 | |
| 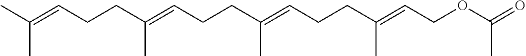 [SMILES: CC(=O)OC/C=C(\C)CC/C=C(\C)CC/C=C(\C)CCC=C(C)C] | | 289 | |
| 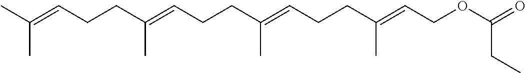 [SMILES: CCC(=O)OC/C=C(\C)CC/C=C(\C)CC/C=C(\C)CCC=C(C)C] | | 147 | |
| 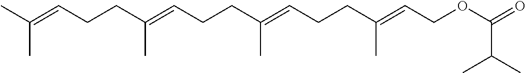 [SMILES: CC(C)=CCC/C(C)=C/CC/C(C)=C/CC/C(C)=C/COC(=O)C(C)C] | | 160 | |
| 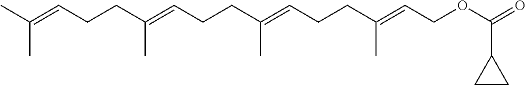 [SMILES: CC(C)=CCC/C(C)=C/CC/C(C)=C/CC/C(C)=C/COC(=O)C1CC1] | | 155 | |
| 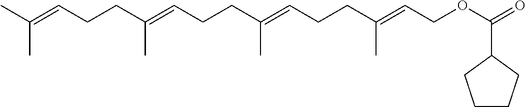 [SMILES: CC(C)=CCC/C(C)=C/CC/C(C)=C/CC/C(C)=C/COC(=O)C1CCCC1] | | 264 | |
| 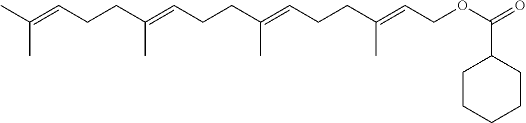 [SMILES: CC(C)=CCC/C(C)=C/CC/C(C)=C/CC/C(C)=C/COC(=O)C1CCCCC1] | | 261 | |
| 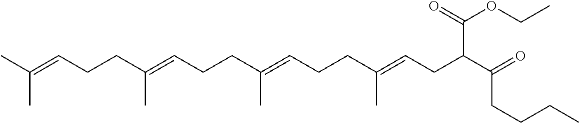 [SMILES: CCCCC(=O)C(C/C=C(\C)CC/C=C(\C)CC/C=C(\C)CCC=C(C)C)C(=O)OCC] | | 243 | |
| 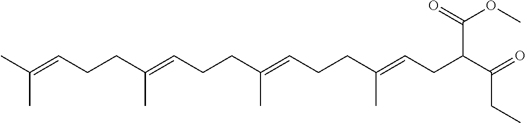 [SMILES: CCC(=O)C(C/C=C(\C)CC/C=C(\C)CC/C=C(\C)CCC=C(C)C)C(=O)OC] | | 212 | |
| 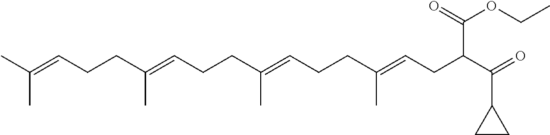 [SMILES: CCOC(=O)C(C/C=C(\C)CC/C=C(\C)CC/C=C(\C)CCC=C(C)C)C(=O)C1CC1] | | 160 | |
| 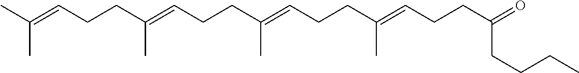 [SMILES: CCCCC(=O)CC/C=C(\C)CC/C=C(\C)CC/C=C(\C)CCC=C(C)C] | | 209 | |
| 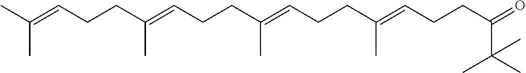 [SMILES: CC(C)=CCC/C(C)=C/CC/C(C)=C/CC/C(C)=C/CCC(=O)C(C)(C)C] | | 198 | |
| 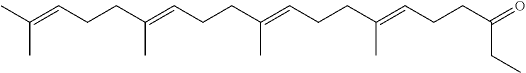 [SMILES: CCC(=O)CC/C=C(\C)CC/C=C(\C)CC/C=C(\C)CCC=C(C)C] | 186 | | |
| 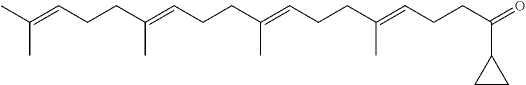 [SMILES: CC(C)=CCC/C(C)=C/CC/C(C)=C/CC/C(C)=C/CCC(=O)C1CC1] | | 180 | |
| 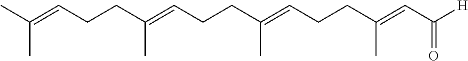 [SMILES: CC(C)=CCC/C(C)=C/CC/C(C)=C/CC/C(C)=C/C=O] | | 174 | |
| 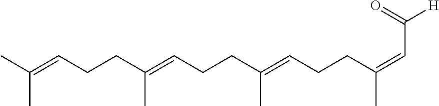 [SMILES: CC(C)=CCC/C(C)=C/CC/C(C)=C/CC/C(C)=C\C=O] | | 199 | |
| 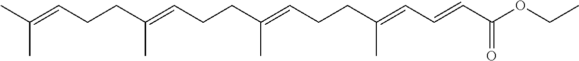 [SMILES: CCOC(=O)/C=C/C=C(\C)CC/C=C(\C)CC/C=C(\C)CCC=C(C)C] | | 200 | |
| 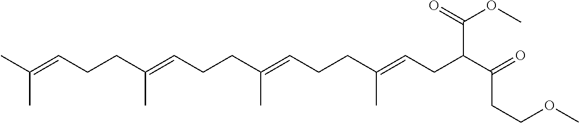 [SMILES: COCCC(=O)C(C/C=C(\C)CC/C=C(\C)CC/C=C(\C)CCC=C(C)C)C(=O)OC] | | 213 | |
| 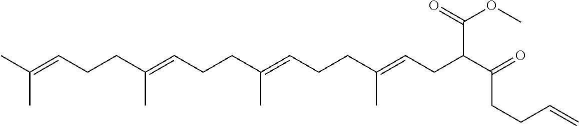 [SMILES: C=CCCC(=O)C(C/C=C(\C)CC/C=C(\C)CC/C=C(\C)CCC=C(C)C)C(=O)OC] | | 162 | |
| 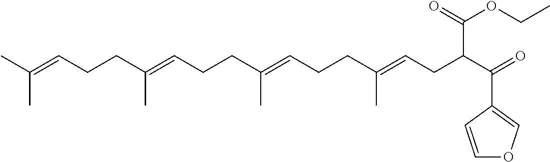 [SMILES: CCOC(=O)C(C/C=C(\C)CC/C=C(\C)CC/C=C(\C)CCC=C(C)C)C(=O)c1ccoc1] | | 152 | |
| 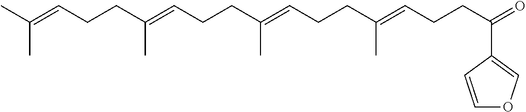 [SMILES: CC(C)=CCC/C(C)=C/CC/C(C)=C/CC/C(C)=C/CCC(=O)c1ccoc1] | | 191 | |
| 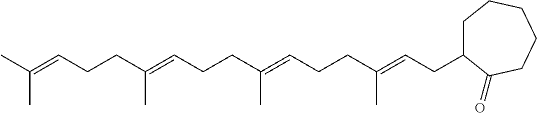 [SMILES: CC(C)=CCC/C(C)=C/CC/C(C)=C/CC/C(C)=C/CC1CCCCCC1=O] | | 151 | |
| 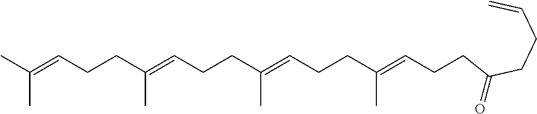 [SMILES: C=CCCC(=O)CC/C=C(\C)CC/C=C(\C)CC/C=C(\C)CCC=C(C)C] | | 206 | |
| 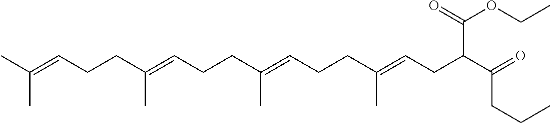 [SMILES: CCCC(=O)C(C/C=C(\C)CC/C=C(\C)CC/C=C(\C)CCC=C(C)C)C(=O)OCC] | | 188 | |
| 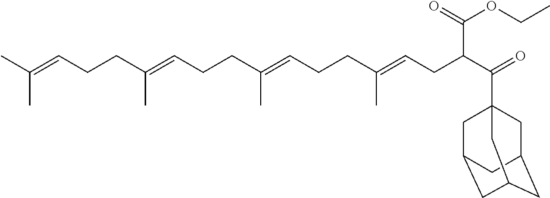 [SMILES: CCOC(=O)C(C/C=C(\C)CC/C=C(\C)CC/C=C(\C)CCC=C(C)C)C(=O)C12CC3CC(CC(C3)C1)C2] | 145 | | |
| 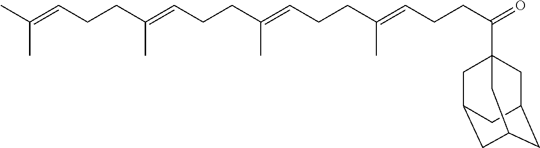 [SMILES: CC(C)=CCC/C(C)=C/CC/C(C)=C/CC/C(C)=C/CCC(=O)C12CC3CC(CC(C3)C1)C2] | | 187 | |
| 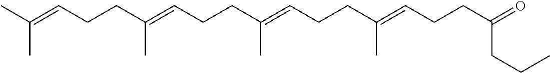 [SMILES: CCCC(=O)CC/C=C(\C)CC/C=C(\C)CC/C=C(\C)CCC=C(C)C] | | 158 | |
| 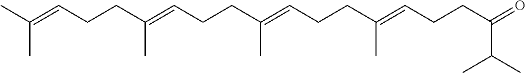 [SMILES: CC(C)=CCC/C(C)=C/CC/C(C)=C/CC/C(C)=C/CCC(=O)C(C)C] | | 165 | |
| 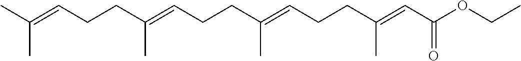 [SMILES: CCOC(=O)/C=C(\C)CC/C=C(\C)CC/C=C(\C)CCC=C(C)C] | | 154 | |
| 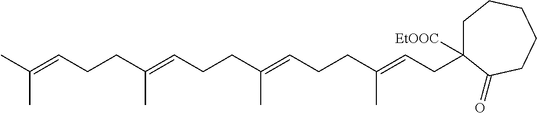 [SMILES: CCOC(=O)C1(C/C=C(\C)CC/C=C(\C)CC/C=C(\C)CCC=C(C)C)CCCCCC1=O] | | 168 | |
| 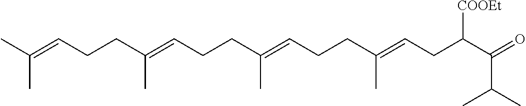 [SMILES: CCOC(=O)C(C/C=C(\C)CC/C=C(\C)CC/C=C(\C)CCC=C(C)C)C(=O)C(C)C] | 173 | | |
| 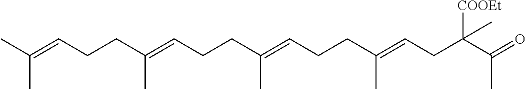 [SMILES: CCOC(=O)C(C)(C/C=C(\C)CC/C=C(\C)CC/C=C(\C)CCC=C(C)C)C(C)=O] | | 145 | |
| 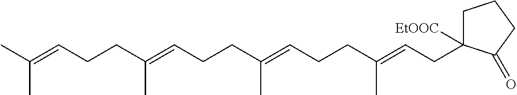 [SMILES: CCOC(=O)C1(C/C=C(\C)CC/C=C(\C)CC/C=C(\C)CCC=C(C)C)CCCC1=O] | | 131 | |
| 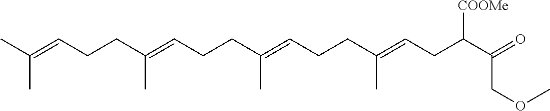 [SMILES: COCC(=O)C(C/C=C(\C)CC/C=C(\C)CC/C=C(\C)CCC=C(C)C)C(=O)OC] | | 167 | |
| 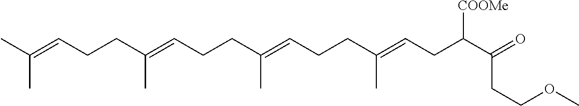 [SMILES: COCCC(=O)C(C/C=C(\C)CC/C=C(\C)CC/C=C(\C)CCC=C(C)C)C(=O)OC] | | 141 | |
| 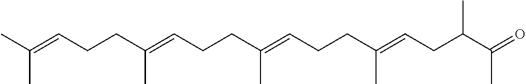 [SMILES: CC(=O)C(C)C/C=C(\C)CC/C=C(\C)CC/C=C(\C)CCC=C(C)C] | | 142 | |
| 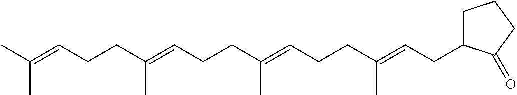 [SMILES: CC(C)=CCC/C(C)=C/CC/C(C)=C/CC/C(C)=C/CC1CCCC1=O] | | 136 | |
| 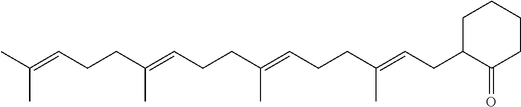 [SMILES: CC(C)=CCC/C(C)=C/CC/C(C)=C/CC/C(C)=C/CC1CCCCC1=O] | | 228 | |
| 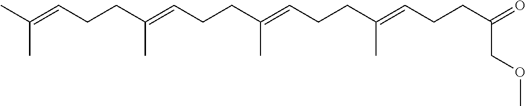 [SMILES: COCC(=O)CC/C=C(\C)CC/C=C(\C)CC/C=C(\C)CCC=C(C)C] | | 158 | |
| 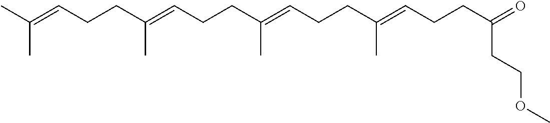 [SMILES: COCCC(=O)CC/C=C(\C)CC/C=C(\C)CC/C=C(\C)CCC=C(C)C] | | 217 | |
| 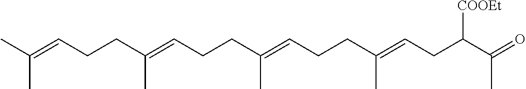 [SMILES: CCOC(=O)C(C/C=C(\C)CC/C=C(\C)CC/C=C(\C)CCC=C(C)C)C(C)=O] | | 185 | |
| 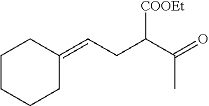 [SMILES: CCOC(=O)C(CC=C1CCCCC1)C(C)=O] | | | 162* |
| 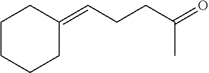 | | 188* | |
| 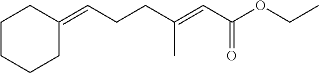 [SMILES: CCOC(=O)/C=C(\C)CCC=C1CCCCC1] | | | 161* |
| 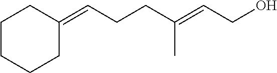 | * Under the conditions tested, this compound was not found to have activity beyond that of the control for this assay. | | |
| 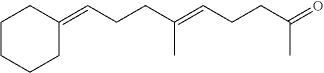 [SMILES: CC(=O)CC/C=C(\C)CCC=C1CCCCC1] | | 149* | |
| 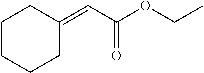 | | | 240* |
| 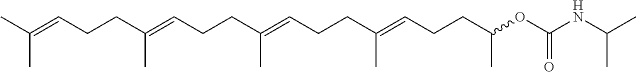 [SMILES: CC(C)=CCC/C(C)=C/CC/C(C)=C/CC/C(C)=C/CCC(C)OC(=O)NC(C)C] | | 189 | |
| 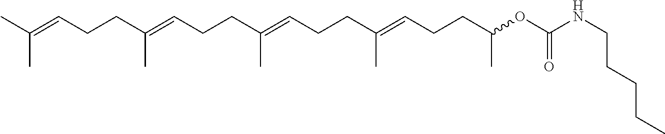 [SMILES: CCCCCNC(=O)OC(C)CC/C=C(\C)CC/C=C(\C)CC/C=C(\C)CCC=C(C)C] | | 200 | |
| 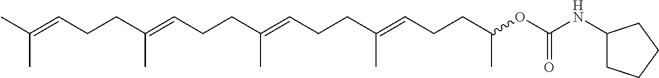 [SMILES: CC(C)=CCC/C(C)=C/CC/C(C)=C/CC/C(C)=C/CCC(C)OC(=O)NC1CCCC1] | | 201 | |
| 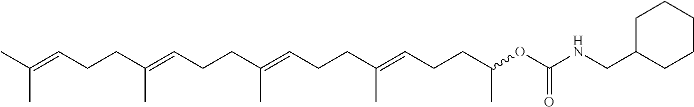 [SMILES: CC(C)=CCC/C(C)=C/CC/C(C)=C/CC/C(C)=C/CCC(C)OC(=O)NCC1CCCCC1] | | 165 | |
| 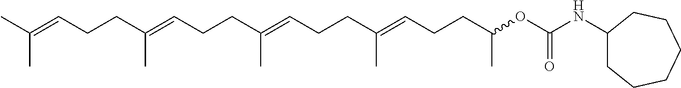 [SMILES: CC(C)=CCC/C(C)=C/CC/C(C)=C/CC/C(C)=C/CCC(C)OC(=O)NC1CCCCCC1] | | 213 | |
| 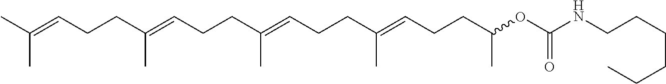 [SMILES: CCCCCCNC(=O)OC(C)CC/C=C(\C)CC/C=C(\C)CC/C=C(\C)CCC=C(C)C] | | 164 | |
| 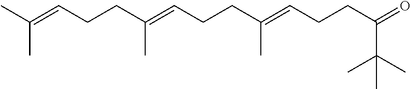 [SMILES: CC(C)=CCC/C(C)=C/CC/C(C)=C/CCC(=O)C(C)(C)C] | | 204 | |
| 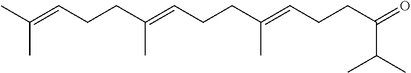 [SMILES: CC(C)=CCC/C(C)=C/CC/C(C)=C/CCC(=O)C(C)C] | | 192 | |
| 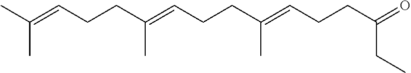 [SMILES: CCC(=O)CC/C=C(\C)CC/C=C(\C)CCC=C(C)C] | | 202 | |
| 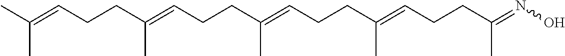 [SMILES: CC(C)=CCC/C(C)=C/CC/C(C)=C/CC/C(C)=C/CCC(C)=NO] | | 178 | |
| 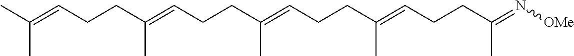 [SMILES: CON=C(C)CC/C=C(\C)CC/C=C(\C)CC/C=C(\C)CCC=C(C)C] | | 218 | |
| 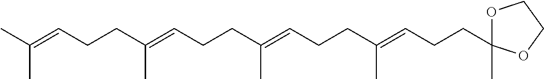 [SMILES: CC(C)=CCC/C(C)=C/CC/C(C)=C/CC/C(C)=C/CCC1(C)OCCO1] | | 252 | |
| 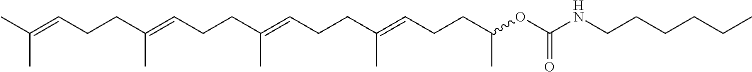 [SMILES: CCCCCCNC(=O)OC(C)CC/C=C(\C)CC/C=C(\C)CC/C=C(\C)CCC=C(C)C] | | 262 | |
| 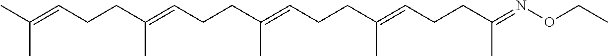 [SMILES: CCO/N=C(\C)CC/C=C(\C)CC/C=C(\C)CC/C=C(\C)CCC=C(C)C] | | 173 | |
| 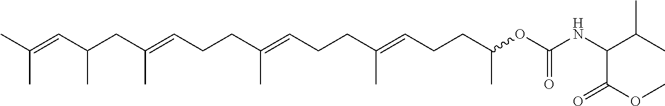 [SMILES: COC(=O)C(NC(=O)OC(C)CC/C=C(\C)CC/C=C(\C)CC/C=C(\C)CC(C)C=C(C)C)C(C)C] | | 200 | |
| 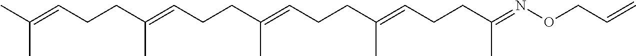 [SMILES: C=CCO/N=C(\C)CC/C=C(\C)CC/C=C(\C)CC/C=C(\C)CCC=C(C)C] | | 214 | |
| 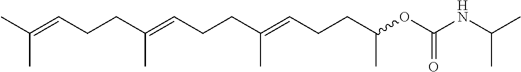 [SMILES: CC(C)=CCC/C(C)=C/CC/C(C)=C/CCC(C)OC(=O)NC(C)C] | | 205 | |
| 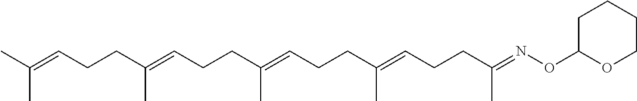 [SMILES: CC(C)=CCC/C(C)=C/CC/C(C)=C/CC/C(C)=C/CC/C(C)=N/OC1CCCCO1] | | 245 | |
| 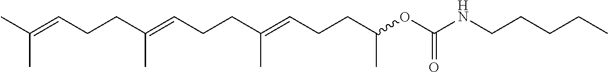 [SMILES: CCCCCNC(=O)OC(C)CC/C=C(\C)CC/C=C(\C)CCC=C(C)C] | | 245 | |
| 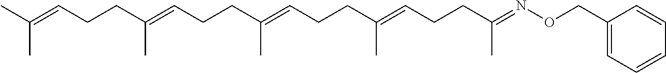 [SMILES: CC(C)=CCC/C(C)=C/CC/C(C)=C/CC/C(C)=C/CC/C(C)=N/OCc1ccccc1] | | | 287 |
| 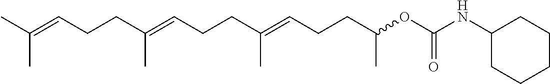 [SMILES: CC(C)=CCC/C(C)=C/CC/C(C)=C/CCC(C)OC(=O)NC1CCCCC1] | | 244 | |
| 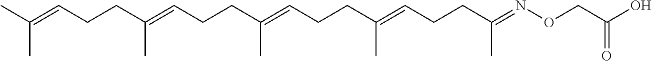 [SMILES: CC(C)=CCC/C(C)=C/CC/C(C)=C/CC/C(C)=C/CC/C(C)=N/OCC(=O)O] | | 264 | |
| 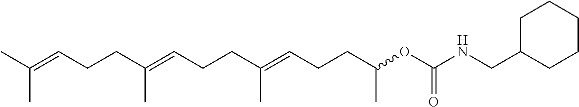 [SMILES: CC(C)=CCC/C(C)=C/CC/C(C)=C/CCC(C)OC(=O)NCC1CCCCC1] | | 229 | |
| 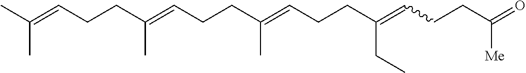 [SMILES: CCC(=CCCC(C)=O)CC/C=C(\C)CC/C=C(\C)CCC=C(C)C] | | 204 | |
| 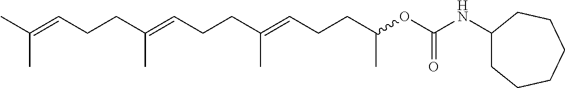 [SMILES: CC(C)=CCC/C(C)=C/CC/C(C)=C/CCC(C)OC(=O)NC1CCCCCC1] | | 176 | |
| 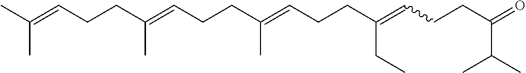 [SMILES: CCC(=CCCC(=O)C(C)C)CC/C=C(\C)CC/C=C(\C)CCC=C(C)C] | | 236 | |
| 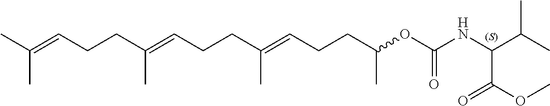 | | 254 | |
| 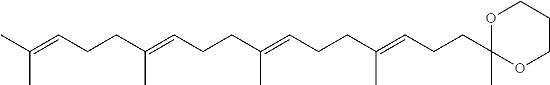 [SMILES: CC(C)=CCC/C(C)=C/CC/C(C)=C/CC/C(C)=C/CCC1(C)OCCCO1] | | 191 | |
| 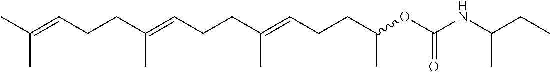 [SMILES: CCC(C)NC(=O)OC(C)CC/C=C(\C)CC/C=C(\C)CCC=C(C)C] | | 229 | |
| 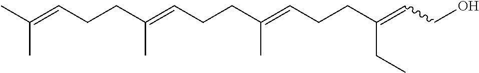 [SMILES: CCC(=CCO)CC/C=C(\C)CC/C=C(\C)CCC=C(C)C] | | 204 | |
| 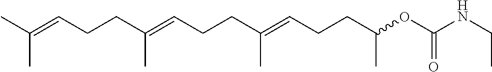 [SMILES: CCNC(=O)OC(C)CC/C=C(\C)CC/C=C(\C)CCC=C(C)C] | | 190 | |
| 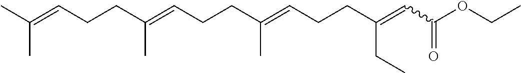 [SMILES: CCOC(=O)C=C(CC)CC/C=C(\C)CC/C=C(\C)CCC=C(C)C] | | 225 | |
| 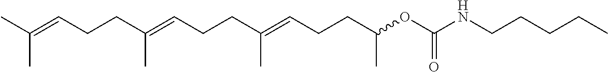 [SMILES: CCCCCNC(=O)OC(C)CC/C=C(\C)CC/C=C(\C)CCC=C(C)C] | | 179 | |
| 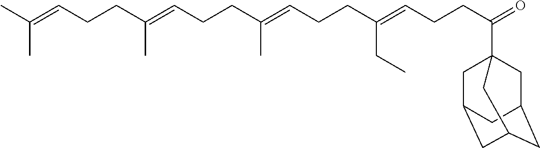 [SMILES: CC/C(=C\CCC(=O)C12CC3CC(CC(C3)C1)C2)CC/C=C(\C)CC/C=C(\C)CCC=C(C)C] | | 179 | |
| 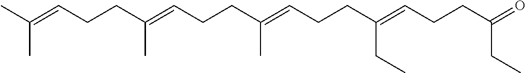 [SMILES: CCC(=O)CC/C=C(\CC)CC/C=C(\C)CC/C=C(\C)CCC=C(C)C] | | 247 | |
| 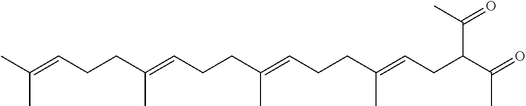 [SMILES: CC(=O)C(C/C=C(\C)CC/C=C(\C)CC/C=C(\C)CCC=C(C)C)C(C)=O] | | 244 | |
| 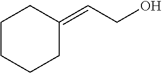 | | 245* | |
| 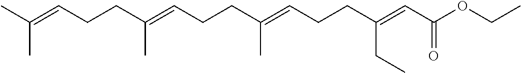 [SMILES: CCOC(=O)/C=C(\CC)CC/C=C(\C)CC/C=C(\C)CCC=C(C)C] | | 196 | |
| 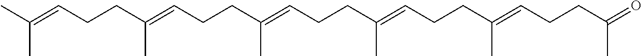 [SMILES: CC(=O)CC/C=C(\C)CC/C=C(\C)CC/C=C(\C)CC/C=C(\C)CCC=C(C)C] | | 274* | |
| 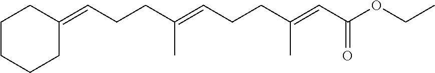 [SMILES: CCOC(=O)/C=C(\C)CC/C=C(\C)CCC=C1CCCCC1] | | 264 | |
| 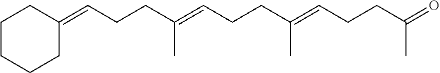 [SMILES: CC(=O)CC/C=C(\C)CC/C=C(\C)CCC=C1CCCCC1] | | 291 | |
| 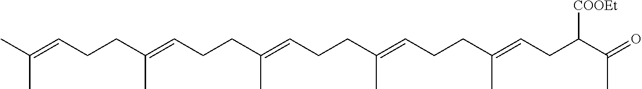 [SMILES: CCOC(=O)C(C/C=C(\C)CC/C=C(\C)CC/C=C(\C)CC/C=C(\C)CCC=C(C)C)C(C)=O] | | 278 | |
| 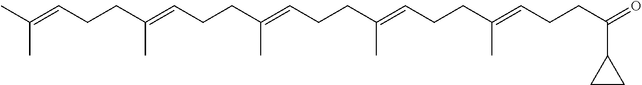 [SMILES: CC(C)=CCC/C(C)=C/CC/C(C)=C/CC/C(C)=C/CC/C(C)=C/CCC(=O)C1CC1] | | 226 | |
| 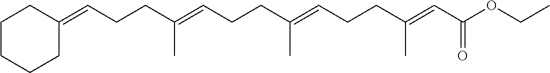 [SMILES: CCOC(=O)/C=C(\C)CC/C=C(\C)CC/C=C(\C)CCC=C1CCCCC1] | | 198 | |
| 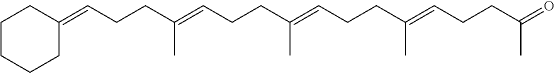 [SMILES: CC(=O)CC/C=C(\C)CC/C=C(\C)CC/C=C(\C)CCC=C1CCCCC1] | | 300 | |
| 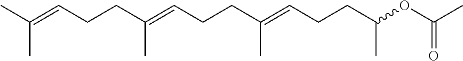 [SMILES: CC(=O)OC(C)CC/C=C(\C)CC/C=C(\C)CCC=C(C)C] | | 238 | |
| 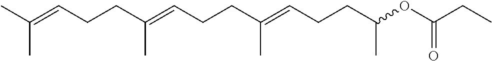 [SMILES: CCC(=O)OC(C)CC/C=C(\C)CC/C=C(\C)CCC=C(C)C] | | 149 | |
| 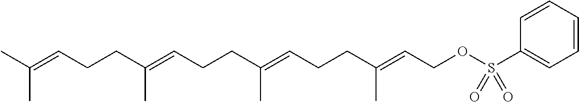 [SMILES: CC(C)=CCC/C(C)=C/CC/C(C)=C/CC/C(C)=C/COS(=O)(=O)c1ccccc1] | | 189 | |
| 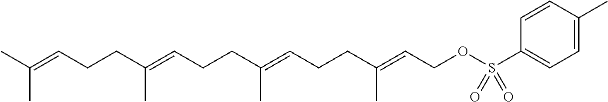 [SMILES: CC(C)=CCC/C(C)=C/CC/C(C)=C/CC/C(C)=C/COS(=O)(=O)c1ccc(C)cc1] | | 224 | |
| 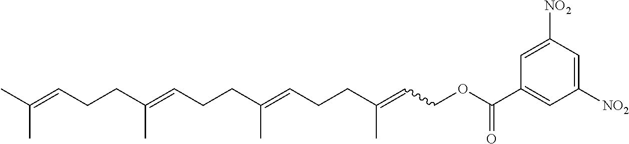 [SMILES: CC(C)=CCC/C(C)=C/CC/C(C)=C/CCC(C)=CCOC(=O)c1cc([N+](=O)[O-])cc([N+](=O)[O-])c1] | | 221 | |
| 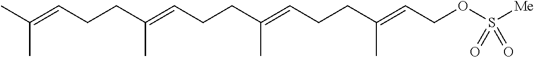 [SMILES: CC(C)=CCC/C(C)=C/CC/C(C)=C/CC/C(C)=C/COS(C)(=O)=O] | | 240 | |

*indicates compounds that are believed to be known, and are useful in the methods of this invention.

The compounds and the compositions of this invention are tested in vivo for their ability to alleviate neurodegenerations induced by Kainic acid. See, for example, PCT Pat. App. No. PCT/US2011/050071, supra. A compound or composition of this invention is orally dosed to Sprague-Dawley rats, and Kainic acid is injected. Seizure behaviors are observed and scored (see, e.g., R. J. Racine, Modification of seizure activity by electrical stimulation: II. Motor seizure, Electroencephalogr. Clin. Neurophysiol. 32 (1972) 281-294). Brain tissues of rats are sectioned on histology slides, and neurons in hippocampus tissues are stained by Nissl.

### Synthesis of GGA Derivatives

The compounds provided herein are synthesized as disclosed herein, following methods well known to the skilled artisan, and/or following methods that will become apparent to the skilled artisan upon reading this disclosure. See, for example, PCT Pat. App. No. PCT/US2011/050071, supra.

The compounds of this invention can be prepared from readily available starting materials using the general methods and procedures described and illustrated herein. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1 15 (John Wiley and Sons, 1991), Rodd's Chemistry of Carbon Compounds, Volumes 1 5 and Supplementals (Elsevier Science Publishers, 1989), Organic Reactions, Volumes 1 40 (John Wiley and Sons, 1991), March's Advanced Organic Chemistry, (John Wiley and Sons, 4^{th} Edition), and Larock's Comprehensive Organic Transformations (VCH Publishers Inc., 1989). For example, the compounds provided herein are synthesized as schematically shown below.

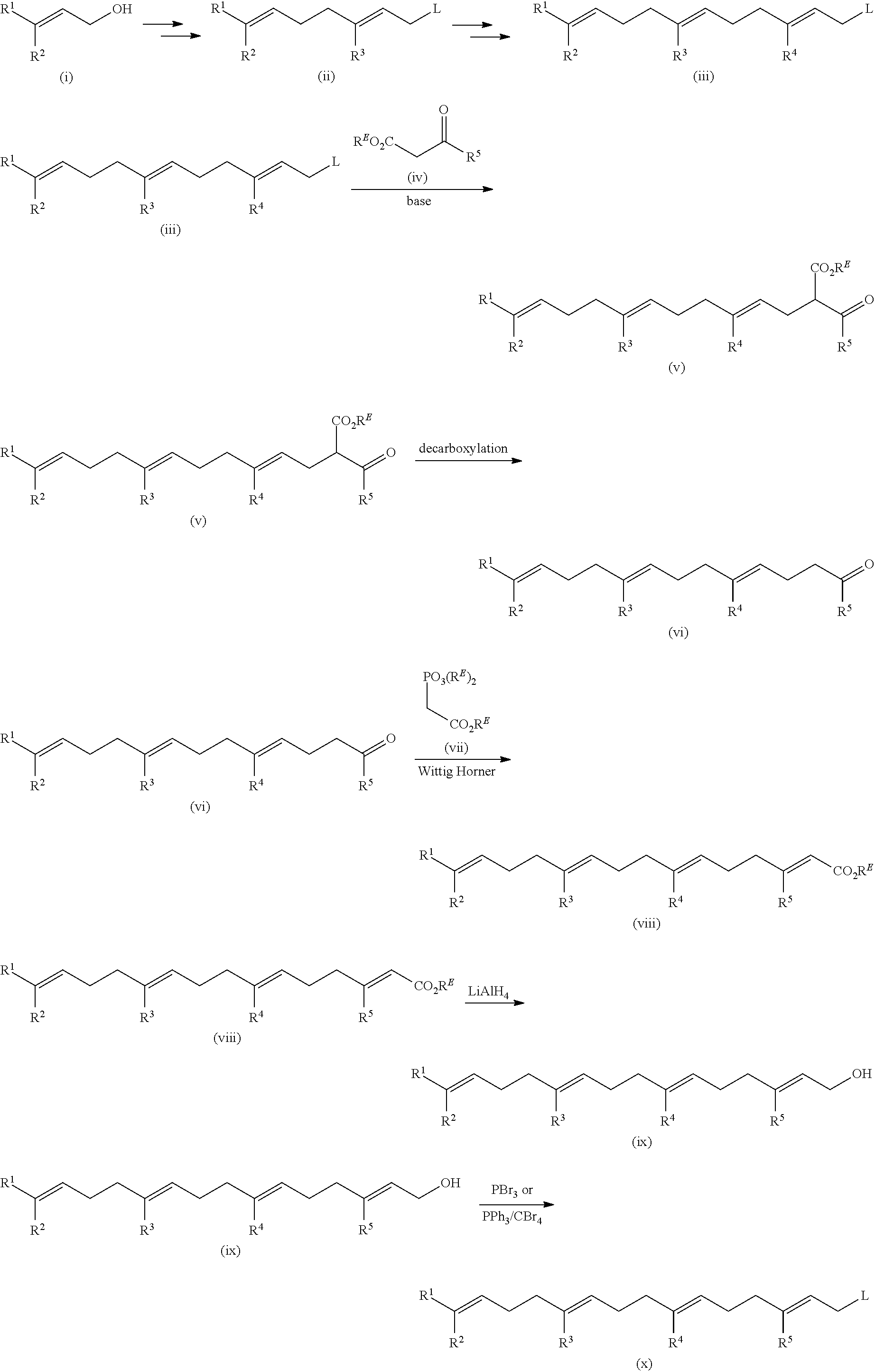

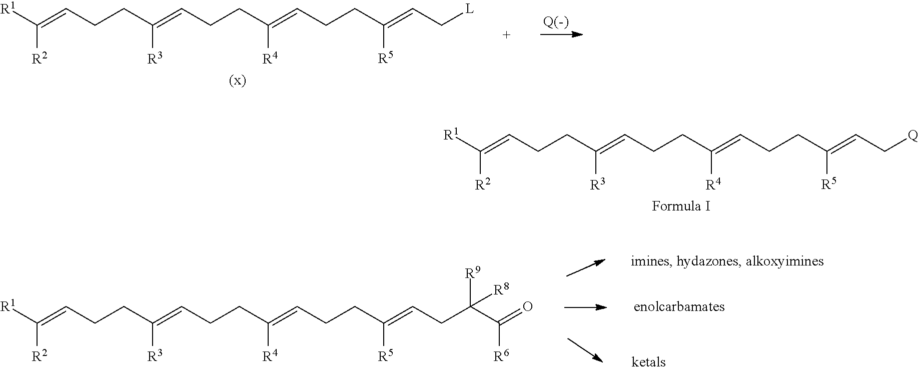

wherein R^{E} is alkyl and L is a leaving group such as chloro, bromo, or iodo, or a sulfonate such as R^{s}SO₂— wherein R^{s} is C₁-C₆ alkyl, C₁-C₆ alkyl substituted with up to 5, preferably up to 3 fluoro atoms, C₆-C₁₀ aryl, or C₆-C₁₀ aryl substituted with a halo or an alkyl group.

Starting compound (iii), which is synthesized from compound (i) by adding isoprene derivatives as described here, is alkylated with a beta keto ester (iv), in the presence of a base such as an alkoxide, to provide compound (v). Compound (v) is hydrolyzed to a carboxylate or a carboxylic acid and thermally decarboxylated to provide compound (vi). Compound (vi) is converted, following a Wittig Horner reaction with compound (vii), to compound (viii). Compound (viii) is reduced, for example with LiAlH₄, to provide compound (ix). Compound (ix) is brominated to provide compound (x). Compound (x), where L is an R^{s}SO₂— group is made by reacting compound (ix) with R^{s}SO₂Cl in the presence of a base. The transformation of compound (iii) to compound (x) illustrates methods of adding isoprene derivatives to a compound, which methods are suitable to make compound (iii) from compound (i).

A compound of Formula I is obtained by reacting compound (x) with the anion Q(−), which can be generated by reacting the compound QH with a base. Suitable nonlimiting examples of bases include hydroxide, hydride, amides, alkoxides, and the like. Various compounds of this invention, wherein the carbonyl group is converted to an imine, a hydrazone, an alkoxyimine, an enolcarbamate, a ketal, and the like, are prepared following well known methods.

Other methods for making the compounds of this invention are schematically illustrated below:

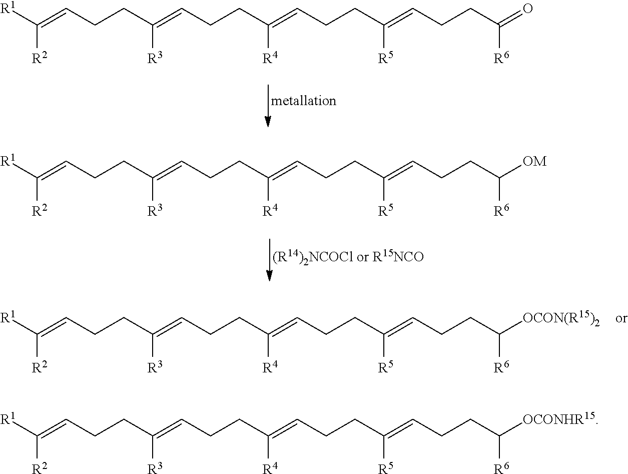

The metallation is performed, by reacting the ketone with a base such as dimsyl anion, a hindered amide base such as diisopropylamide, or hexamethyldisilazide, along with the corresponding metal cation, M. The amino carbonyl chloride or the isocyanate is prepared, for example, by reacting the amine (R¹⁴)₂NH with phosgene or an equivalent reagent well known to the skilled artisan.

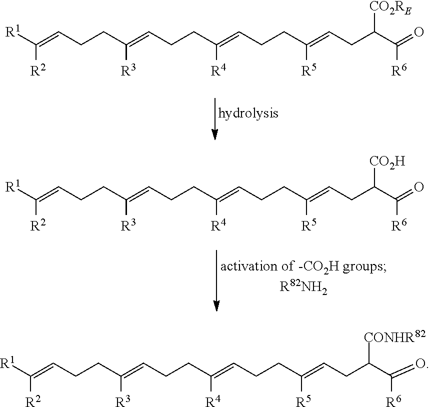

The beta keto ester is hydrolyzed while ensuring that the reaction conditions do not lead to decarboxylation. The acid is activated with various acid activating agent well known to the skilled artisan such as carbonyl diimidazole, or O-Benzotriazole-N,N,N′,N′-tetramethyl-uronium-hexafluoro-phosphate (HBTU) and reacted with the amine.

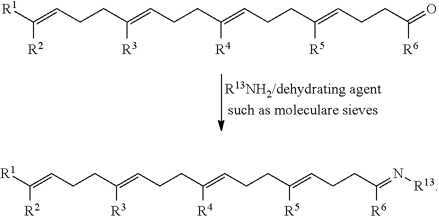

Various other compounds of this invention are prepared as illustrated in the non limiting examples herein below. Still other compounds of this invention are prepared from the compounds made in the schemes above, based on art known methods.

### Pharmaceutical Compositions

In another aspect, this invention provides a composition comprising a GGA derivative provided herein, such as for example, and without limitation, a compound of Formulas (I) and (Ia), and a pharmaceutically acceptable excipient.

Such compositions can be formulated for different routes of administration. Although compositions suitable for oral delivery will probably be used most frequently, other routes that may be used include transdermal, intravenous, intraarterial, pulmonary, rectal, nasal, vaginal, lingual, intramuscular, intraperitoneal, intracutaneous, intracranial, and subcutaneous routes. Suitable dosage forms for administering the GGA derivatives of this invention include tablets, capsules, pills, powders, aerosols, suppositories, parenterals, and oral liquids, including suspensions, solutions and emulsions. Sustained release dosage forms may also be used, for example, in a transdermal patch form. All dosage forms may be prepared using methods that are standard in the art (see e.g., Remington's Pharmaceutical Sciences, 16^{th} ed., A. Oslo editor, Easton Pa. 1980).

Pharmaceutically acceptable excipients are non-toxic, aid administration, and do not adversely affect the therapeutic benefit of the compound of this invention. Such excipients may be any solid, liquid, semi-solid or, in the case of an aerosol composition, gaseous excipient that is generally available to one of skill in the art. Pharmaceutical compositions in accordance with the invention are prepared by conventional means using methods known in the art.

The compositions disclosed herein may be used in conjunction with any of the vehicles and excipients commonly employed in pharmaceutical preparations, e.g., talc, gum arabic, lactose, starch, magnesium stearate, cocoa butter, aqueous or non-aqueous solvents, oils, paraffin derivatives, glycols, etc. Coloring and flavoring agents may also be added to preparations, particularly to those for oral administration. Solutions can be prepared using water or physiologically compatible organic solvents such as ethanol, 1,2-propylene glycol, polyglycols, dimethylsulfoxide, fatty alcohols, triglycerides, partial esters of glycerin and the like.

Solid pharmaceutical excipients include starch, cellulose, hydroxypropyl cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. In certain embodiments, the compositions provided herein comprises one or more of α-tocopherol, gum arabic, and/or hydroxypropyl cellulose.

In one embodiment, this invention provides sustained release formulations such as drug depots or patches comprising an effective amount of a compound provided herein. In another embodiment, the patch further comprises gum Arabic or hydroxypropyl cellulose separately or in combination, in the presence of alpha-tocopherol. Preferably, the hydroxypropyl cellulose has an average MW of from 10,000 to 100,000. In a more preferred embodiment, the hydroxypropyl cellulose has an average MW of from 5,000 to 50,000.

Compounds and pharmaceutical compositions of this invention maybe used alone or in combination with other compounds. When administered with another agent, the co-administration can be in any manner in which the pharmacological effects of both are manifest in the patient at the same time. Thus, co-administration does not require that a single pharmaceutical composition, the same dosage form, or even the same route of administration be used for administration of both the compound of this invention and the other agent or that the two agents be administered at precisely the same time. However, co-administration will be accomplished most conveniently by the same dosage form and the same route of administration, at substantially the same time. Obviously, such administration most advantageously proceeds by delivering both active ingredients simultaneously in a novel pharmaceutical composition in accordance with the present invention.

### Treatment Methods

This invention provides methods for inhibiting neural death and increasing neural activity. For example, and without limitation, the invention provides methods for impeding the progression of neurodegenerative diseases or injury. The pharmaceutical compositions and/or compounds described above are useful in the methods described herein. The compounds provided herein can be co-administered with memantine or Aricept, wherein the memantine or Aricept are administered as separate active ingredients. For co-administration, the compound of this invention and memantine or Aricept can be included in the same composition or can be administered as separate compositions. The compound of this invention and memantine or Aricept can be administered at the same time or at different times.

In one aspect, provided herein are methods for increasing the axon growth of neurons by contacting said neurons with an effective amount of a compound provided herein. Neural diseases can result in an impairment of signaling between neurons. This can in part be due to a reduction in the growth of axonal projections. Contacting neurons with the compounds or compositions provided herein enhances axonal growth. It is contemplated that the compounds or compositions provided herein will restore axonal grown in neurons afflicted with a neural disease. In a related embodiment, the pre-contacted neurons exhibit a reduction in the axon growth ability.

Another aspect of this invention is directed to a method for inhibiting the cell death of neurons susceptible to neuronal cell death, which method comprises contacting said neurons with an effective amount of a compound or a composition provided herein. Neurons susceptible to neuronal cell death include those that have the characteristics of a neurodegenerative disease and/or those that have undergone injury or toxic stress. One method of creating toxic stress to a cell is by mixing dopamine with neurons such as neuroblastoma cells. Another source of toxic stress is oxidative stress. Oxidative stress can occur from neuronal disease or injury. It is contemplated that contacting neurons with a compound provided herein will inhibit their death as measured by a MTT assay or other techniques commonly known to one skilled in the art.

In another aspect, provided herein are methods for increasing the neurite growth of neurons by contacting said neurons with an effective amount of a compound or a composition provided herein. The term “neurite” refers to both axons and dendrites. Neural diseases can result in an impairment of signaling between neurons. This can in part be due to a reduction in the growth of axonal and/or dendritic projections. It is contemplated that contacting neurons with a compound provided herein will enhance neurite growth. It is further contemplated that a compound provided herein will restore neurite grown in neurons afflicted with a neural disease. In a related embodiment, the pre-contacted neurons exhibit a reduction in the neurite growth ability.

In one specific embodiment of the methods disclosed herein, the compound is selected from the group consisting of

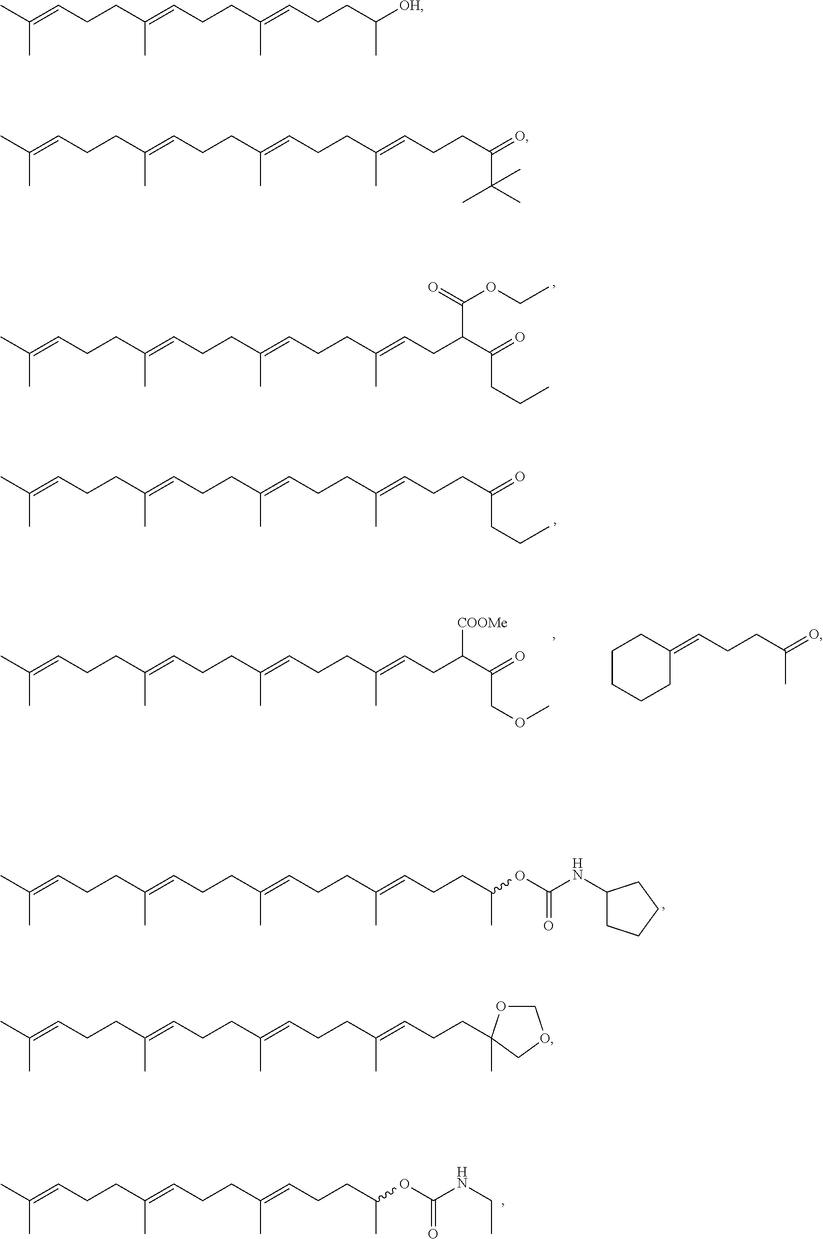

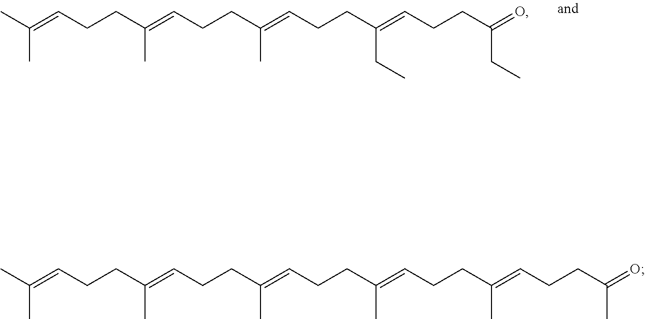

wherein the effective amount of the compound contacting the cell is less than about 1 μM. In a related embodiment, the effective amount is less than about 100 nM. Certain compounds of the invention exhibit a decrease in activity above a certain concentration. Accordingly, these compounds may be more efficacious at lower doses.

Another aspect of this invention is directed to a method for increasing the expression and/or release of one or more neurotransmitters from a neuron by contacting said neurons with an effective amount of a compound or a composition provided herein. It is contemplated that contacting neurons with an effective amount of a compound provided herein will increase the expression level of one or more neurotransmitters. It is also contemplated that contacting neurons with a compound provided herein will increase the release of one or more neurotransmitters from neurons. The release of one or more neurotransmitters refers to the exocytotic process by which secretory vesicles containing one or more neurotransmitters are fused to cell membrane, which directs the neurotransmitters out of the neuron. It is contemplated that the increase in the expression and/or release of neurotransmitters will lead to enhanced signaling in neurons, in which levels of expression or release of neurotransmitters are otherwise reduced due to the disease. The increase in their expression and release can be measured by molecular techniques commonly known to one skilled in the art.

Another aspect of this invention is directed to a method for inducing synapse formation of a neuron by contacting said neurons with an effective amount of a compound or a composition provided herein. A synapse is a junction between two neurons. Synapses are essential to neural function and permit transmission of signals from one neuron to the next. Thus, an increase in the neural synapses will lead to an increase in the signaling between two or more neurons. It is contemplated that contacting the neurons with an effective amount of a compound provided herein will increase synapse formation in neurons that otherwise experience reduced synapse formation as a result of neural disease.

Another aspect of this invention is directed to a method for increasing electrical excitability of a neuron by contacting said neurons with an effective amount of a compound or a composition provided herein. Electrical excitation is one mode of communication among two or more neurons. It is contemplated that contacting neurons with an effective amount of a compound provided herein will increase the electrical excitability of neurons in which electrical excitability and other modes of neural communication are otherwise impaired due to neural disease. Electrical excitability can be measured by electrophysiological methods commonly known to one skilled in the art.

In each of the three previous paragraphs above, the administration of a compound or a composition provided herein enhances communication between neurons and accordingly provides for a method of inhibiting the loss of cognitive abilities in a mammal that is at risk of dementia or suffering from incipient or partial dementia while retaining some cognitive skills. Incipient or partial dementia in a mammal is one in which the mammal still exhibits some cognitive skills, but the skills are being lost and/or diminished over time.

In another aspect, this invention is directed to a method for inhibiting the death of neurons due to formation of or further formation of pathogenic protein aggregates between, outside or inside neurons, wherein said method comprises contacting said neurons at risk of developing said pathogenic protein aggregates with an amount of a compound or a composition provided herein inhibitory to protein aggregate formation, provided that said pathogenic protein aggregates are not related to SBMA. In one embodiment of this invention, the pathogenic protein aggregates form between or outside of the neurons. In another embodiment of this invention, the pathogenic protein aggregates form inside said neurons. In one embodiment of this invention, the pathogenic protein aggregates are a result of toxic stress to the cell. One method of creating toxic stress to a cell is by mixing dopamine with neurons such as neuroblastoma cells. It is contemplated that contacting neurons with a compound provided herein will inhibit their death as measured by a MTT assay or other techniques commonly known to one skilled in the art.

Another aspect of the invention is directed to a method for protecting neurons from pathogenic extracellular protein aggregates which method comprises contacting said neurons and/or said pathogenic protein aggregates with an amount of a compound provided herein that inhibits further pathogenic protein aggregation. In one embodiment of this invention, contacting said neurons and/or said pathogenic protein aggregates with an effective amount of a compound provided herein alters the pathogenic protein aggregates into a non-pathogenic form. Without being limited to any theory, it is contemplated that contacting the neurons and/or the pathogenic protein aggregates with a compound provided herein will solubilize at least a portion of the pathogenic protein aggregates residing between, outside, or inside of the cells. It is further contemplated that contacting the neurons and/or the pathogenic protein aggregates with a compound provided herein will alter the pathogenic protein aggregates in such a way that they are non-pathogenic. A non-pathogenic form of the protein aggregate is one that does not contribute to the death or loss of functionality of the neuron. There are many assays known to one skilled in the art for measuring the protection of neurons either in cell culture or in a mammal. One example is a measure of increased cell viability by a MTT assay. Another example is by immunostaining neurons in vitro or in vivo for cell death-indicating molecules such as, for example, caspases or propidium iodide.

In another embodiment, this invention provides a method for protecting neurons from pathogenic intracellular protein aggregates which method comprises contacting said neurons with an amount of a compound provided herein which will inhibit further pathogenic protein aggregation provided that said protein aggregation is not related to SBMA. This method is not intended to inhibit or reduce, negative effects of neural diseases in which the pathogenic protein aggregates are intranuclear or diseases in which the protein aggregation is related to SBMA. SBMA is a disease caused by pathogenic androgen receptor protein accumulation. It is distinct from the neural diseases mentioned in this application since the pathogenic protein aggregates of SBMA contain polyglutamines and are formed intranuclearly. It is also distinct from the neural diseases described in this application because the protein aggregates are formed from androgen receptor protein accumulation. It is contemplated that contacting neurons with an effective amount of a compound provided herein will alter the pathogenic protein aggregate into a non-pathogenic form.

One embodiment of the invention is directed to a method of modulating the activity of G proteins in neurons which method comprises contacting said neurons with an effective amount of a compound provided herein. It is contemplated that contacting neurons with GGA will alter the sub-cellular localization, thus changing the activities of the G protein in the cell. In one embodiment of the invention, contacting neurons with a compound provided herein will enhance the activity of G proteins in neurons. It is contemplated that contacting a compound provided herein with neurons will increase the expression level of G proteins. It is also contemplated that contacting a compound provided herein with neurons will enhance the activity of G proteins by changing their sub-cellular localization to the cell membranes where they must be to exert their biological activities.

One embodiment of the invention is directed to a method of modulating or enhancing the activity of G proteins in neurons at risk of death which method comprises contacting said neurons with an effective amount of a compound provided herein. Neurons may be at risk of death as a result of genetic changes related to ALS. One such genetic mutation is a depletion of the TDP-43 protein. It is contemplated that neurons with depleted TDP-43 or other genetic mutations associated with ALS will have an increase or change in the activity of G proteins after being contacted with a compound provided herein. It is further contemplated that a compound provided herein will result in an increase in the activity of G proteins in these cells by changing their sub-cellular localization to the cell membranes where they must be to exert their biological activities.

Another aspect of the invention is directed to a method for inhibiting the neurotoxicity of β-amyloid peptide by contacting the β-amyloid peptide with an effective amount of a compound provided herein. In one embodiment of the invention the β-amyloid peptide is between or outside of neurons. In yet another embodiment of the invention, the β-amyloid peptide is part of the β-amyloid plaque. It is contemplated that contacting neurons with a compound provided herein will result in solubilizing at least a portion of the β-amyloid peptide, thus decreasing its neurotoxicity. It is further contemplated that a compound provided herein will decrease the toxicity of the β-amyloid peptide by altering it in such a way that it is no longer toxic to the cell.

Compounds disclosed herein are useful for inducing heat shock proteins. Example 3 demonstrates the induction of heat shock proteins by compounds disclosed herein. The induction of HSPs can be in vitro in cultured cells or in vivo in a subject such as, for example, a rat, a mouse, or a human. Accordingly, one aspect of this invention relates to a method for increasing the expression of a heat shock protein in a cell comprising contacting the cell with a compound disclosed herein. Another aspect of the invention relates to a method for increasing the expression of a heat shock protein or mRNA in a subject in need thereof comprising administering to the subject an effective amount of a compound or composition disclosed herein. An effective amount is one that provides for a therapeutic induction of HSPs in the cell or subject. In certain embodiments, the HSP is HSP70. In further embodiments, the HSP70 mRNA or protein is increased by at least 4%. In a preferred embodiment, HSP70 mRNA or protein is increased by about 15%. In other embodiments, HSP70 is induced by about 6%, 8%, 10%, 12%, 14%, 16%, 18%, 20%, 25%, 30%, or 40%. The induced heat shock proteins in the neurons or glial cells may be transmitted extracellularly and act to dissolve extracellular protein aggregates. Cell viability can be measured by standard assays known to those skilled in the art. One such example of an assay to measure cell viability is a MTT assay. Another example is a MTS assay. The modulation of protein aggregation can be visualized by immunostaining or histological staining techniques commonly known to one skilled in the art.

One embodiment of the invention is directed to a method for inhibiting neural death and increasing neural activity in a mammal suffering from neural diseases, wherein the etiology of said neural diseases comprises formation of protein aggregates which are pathogenic to neurons, and which method comprises administering to said mammal an amount of a compound provided herein which will inhibit further pathogenic protein aggregation. This method is not intended to inhibit neural death and increase neural activity in neural diseases in which the pathogenic protein aggregates are intranuclear or diseases in which the protein aggregation is related to SBMA.

Neural diseases such as AD and ALS disease have the common characteristic of protein aggregates either inside neural cells in cytoplasm or in the extracellular space between two or more neural cells. This invention also relates to a method for using a compound provided herein to inhibit the formation of the protein aggregates or alter the pathogenic protein aggregates into a non-pathogenic form. It is contemplated that this will attenuate some of the symptoms associated with these neural diseases.

In one aspect the mammal is a human afflicted with a neural disease. In one embodiment of this invention, the negative effect of the neural disease being inhibited or reduced is ALS. ALS is characterized by a loss of functionality of motor neurons. This results in the inability to control muscle movements. ALS is a neurodegenerative disease that does not typically show intranuclear protein aggregates. It is contemplated that a compound provided herein will prevent or inhibit the formation of extracellular or intracellular protein aggregates that are cytoplasm, not intranuclear and not related to SBMA. It is also contemplated that a compound provided herein will alter the pathogenic protein aggregates into a form that is non-pathogenic. Methods for diagnosing ALS are commonly known to those skilled in the art. Additionally, there are numerous patents that describe methods for diagnosing ALS. These include U.S. Pat. No. 5,851,783 and U.S. Pat. No. 7,356,521 both of which are incorporated herein by reference in their entirety.

In one aspect of this invention, the negative effect of the neural disease being inhibited or reduced is that resulting from AD. AD is a neurodegenerative disease that does not typically show intranuclear protein aggregates. It is contemplated that GGA will prevent or inhibit the formation of extracellular or intracellular protein aggregates. It is also contemplated that GGA will alter the pathogenic protein aggregates into a form that is non-pathogenic. Methods for diagnosing AD are commonly known to those skilled in the art. Additionally, there are numerous patents that describe methods for diagnosing AD. These include U.S. Pat. No. 6,130,048 and U.S. Pat. No. 6,391,553 both of which are incorporated herein by reference in their entirety.

In another embodiment, the mammal is a laboratory research mammal such as a mouse. In one embodiment of this invention, the neural disease is ALS. One such mouse model for ALS is a transgenic mouse with a Sod1 mutant gene. It is contemplated that GGA will enhance the motor skills and body weights when administered to a mouse with a mutant Sod1 gene. It is further contemplated that administering a compound provided herein to this mouse will increase the survival rate of Sod1 mutant mice. Motor skills can be measured by standard techniques known to one skilled in the art. In yet another embodiment of this invention, the neural disease is AD. One example of a transgenic mouse model for AD is a mouse that overexpresses the APP (Amyloid beta Precursor Protein). It is contemplated that administering GGA to a transgenic AD mouse will improve the learning and memory skills of said mouse. It is further contemplated that GGA will decrease the amount and/or size of β-amyloid peptide and/or plaque found inside, between, or outside of neurons. The β-amyloid peptide or plaque can be visualized in histology sections by immunostaining or other staining techniques.

In one embodiment of the invention, administering a compound provided herein to a mammal alters the pathogenic protein aggregate present into a non-pathogenic form. In another embodiment of the invention, administering a compound provided herein to a mammal will prevent pathogenic protein aggregates from forming.

Another aspect of this invention relates to a method for reducing seizures in a mammal in need thereof, which method comprises administering a therapeutically effective amount of a compound provided herein, thereby reducing seizures. The reduction of seizures refers to reducing the occurrence and/or severity of seizures. In one embodiment, the seizure is epileptic seizure. In another embodiment, the methods of this invention prevent neural death during epileptic seizures. The severity of the seizure can be measured by one skilled in the art.

In certain aspects, the methods described herein relate to administering a compound provided herein in vitro. In other aspects the administration is in vivo. In yet other aspects, the in vivo administration is to a mammal. Mammals include but are not limited to humans and common laboratory research animals such as, for example, mice, rats, dogs, pigs, cats, and rabbits.

As used herein, compounds provided herein include compounds provided in various compounds aspects and embodiments herein. It is contemplated that the compounds excluded from the compounds of Formula (I) are also useful in the various treatment method and pharmaceutical composition aspects and embodiments provided herein.

### EXAMPLES

### Example 1

### Synthesis of GGA Derivatives

Synthesis of various GGA derivatives are illustrated herein below. In the examples, the compounds are not necessarily numbered consecutively.

### 2E,6E-Farnesyl Bromide (2)

To a stirred solution of 2E,6E-Farnesyl alcohol 3 (6.9 g, 31.08 mmol) in diethyl ether (80 mL) at 0° C. was added phosphorus tribromide (0.95 mL, 10.25 mmol) dropwise over several minutes. The reaction mixture was further stirred at 0° C. for an additional 1 h. The reaction was quenched with water (5 mL), the diethyl ether was removed under a reduced pressure and the resulting slurry was suspended in water (100 mL). The aqueous slurry was the extracted with n-hexanes (3×150 mL), dried over anhydrous Na₂SO₄ and solvent was evaporated to obtain the desired bromide 2. Yield: 8.7 g (Crude); TLC Rf: 0.93 (10% EtOAc in n-Hexanes); Since this bromide 4 was found to be unstable for a silica gel column chromatography, it was used as such without any purification in the next step.

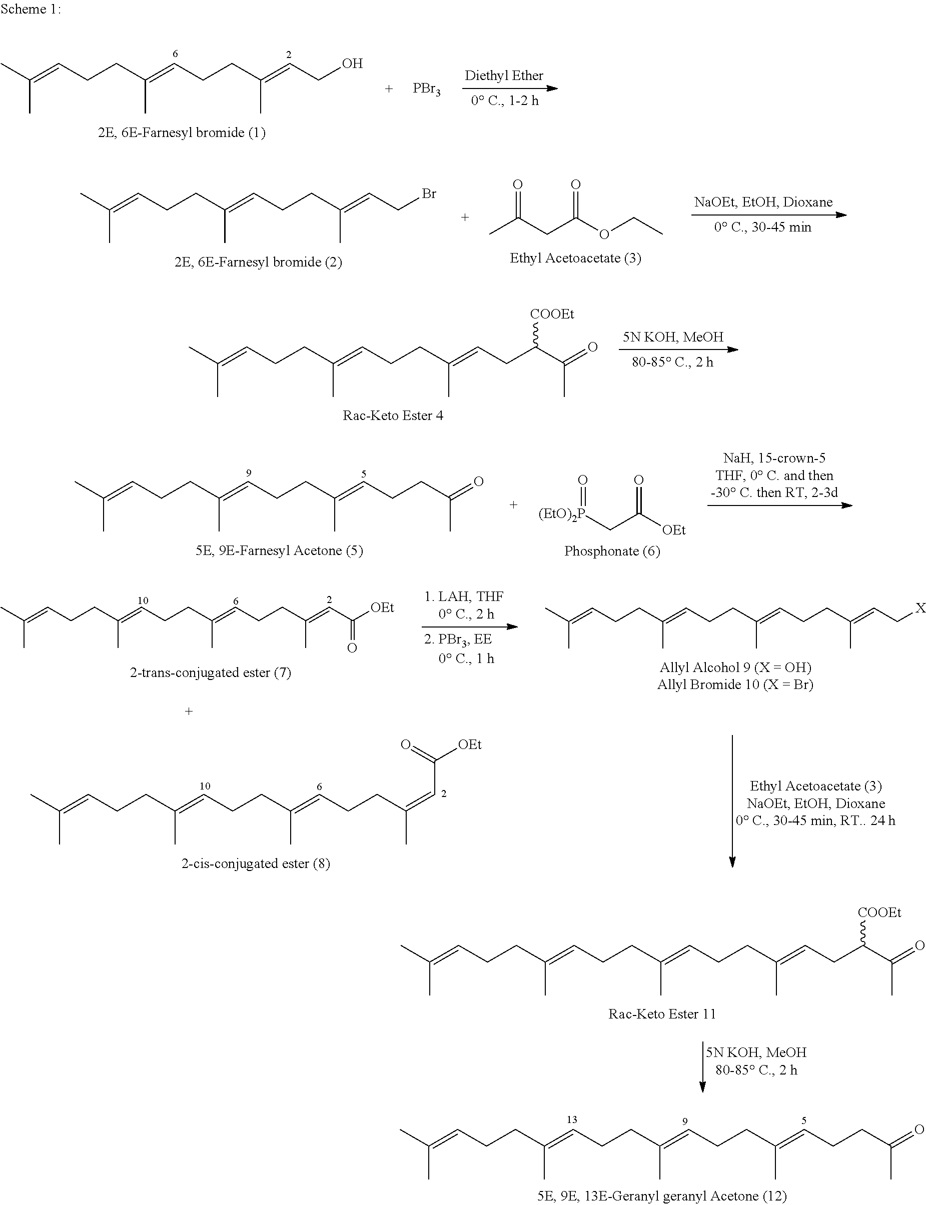

### 5E,9E-Farnesyl-rac-3-carboethoxy acetone (4)

To a solution of sodium ethoxide (13.86 mL, 42.88 mmol; 21% solution in EtOH) in ethanol (15 mL) at 0° C. was added ethyl acetoacetate (3) over a period of 5 minutes and stirred at the same temperature for 20 minutes. To it at 0° C. was added a solution of bromide 2 (8.7 g, 30.6 mmol) in dioxane (15 mL) dropwise over 5-10 minutes. The resulting reaction mixture was allowed to come to room temperature and then stirred for overnight. The reaction mixture was diluted with n-hexanes (200 mL), the organic phase was washed with water (3×50 mL), dried over anhydrous Na₂SO₄ and solvent was evaporated under a reduced pressure to obtain 11 g of ketoester 4 as a oily residue. The resulting oily product had unknown amount of ethyl acetoacetate (3) and other by-products, which was purified by a column chromatography (silica gel, hexanes then 1-3% EtOAc in n-hexanes) to yield a colorless liquid of ketoester 4. Yield: 8.7 g (88%); TLC Rf: 0.36 (5% EtOAc in n-hexanes). LCMS: MS (m/z): 357 (M+Na), 335 (MH⁺).

### 5E,9E-Farnesyl Acetone 5

To a solution of ketoester 4 (6.68 g, 20 mmol) in MeOH (25 mL) at room temperature was added aqueous 5N KOH (14 mL) solution and then stirred at 80° C. for 2.5 h. Upon cooling, the reaction was acidified with 2N HCl until pH3-4 and extracted with EtOAc (3×250 mL). The combined organic phases were washed with H₂O (2×100 mL), saturated aqueous NaHCO₃ (2×100 mL) and finally with H₂O (100 mL). After drying over anhydrous Na₂SO₄, the solvent was removed under a reduced pressure to obtain an oily residue, which was purified by column chromatography (silica gel, hexanes then 1%, 3%, 5% EtOAc in n-hexanes) to afford the desired ketone 5 as a colorless liquid. Yield: 3.4 g; TLC Rf: 0.40 (5% EtOAc in n-hexanes); LCMS: MS (m/z): 263 (MH⁺).

### trans-2E,6E,10E-Conjugated Ester 7

A dry reaction flask equipped with a magnetic stirring bar, N₂ inlet and rubber septum was charged with NaH (60% disp. in oil; 0.584 g, 6.36 mmol), 15-crown-5 (0.1 mL) and anhydrous THF (20 mL). The resulting suspension was cooled 0° C. and to it was added triethyl phoponoacetoacetate 6 (3.49 g, 17.63 mmol) carefully and dropwise. As the addition of 6 was in progress the heterogeneous material was turning clear and became completely clear after the addition was completed. The resulting clear solution was stirred for another 15 minutes and then was cooled to −30° C. To it was added the ketone 5 (3.3 g, 12.5 mmol) as a THF (20 mL) solution over a period of 15-20 minutes. The resulting mixture was allowed to warm to the room temperature and then stirred at RT for 2 days. After quenching the reaction with water (50 mL) carefully, the THF layer was separated; the aqueous layer was extracted with n-hexanes (3×100 mL) and combined with THF layer. The combined organic phases were dried over Na₂SO₄ and solvent was removed under a reduced pressure to afford an oily material, which was purified by silica gel column chromatography using n-hexanes to 1% EtOAc in hexanes. The fast moving product with TLC Rf: 0.68 (5% EtOAc/Hexanes) was identified as cis-isomer 8 and was found to be a very minor product. Yield: 0.3 g, 7%. The next product isolated was identified as trans-isomer 7. Yield: 3.6 g, 90%. TLC Rf: 0.60 (5% EtOAc/n-hexanes); LCMS: MS (m/z): 333 (MH+).

### trans-Allylic Alcohol 9

To a dry reaction flask was placed trans-conjugated ester 7 (1.87 g, 5.6 mmol) and THF (20 mL). At 0° C., under a N₂ atmosphere (with a vent) was added LAH (2M solution in THF, 2.82 mL, 5.6 mmol) drop wise with cautions over 30 min. The resulting reaction was then stirred for additional 2 h at 0° C., which was monitored by TLC. Once the reaction was completed, it was quenched with EtOAc (5 mL) followed by H₂O (4 mL) very carefully, since it generated gaseous hydrogen. The resulting jelly obtained was diluted with EtOAc (100 mL), the solid mass was filtered through celite and washed the celite pad with EtOAc (2×50 mL). The combined organic layers were dried over anhydrous Na₂SO₄ and solvent was removed under a reduced pressure, dried under high vacuum to afford 1.54 g (94%) of the desired alcohol 9. TLC Rf: 0.19 (10% EtOAc/n-hexanes); LCMS: MS (m/z): 291 (MH+).

### trans-Allylic Bromide 10

To a stirred solution of alcohol 9 (2.32 g, 7.9 mmol) in diethyl ether (15 mL) under N2 at 0° C. was added phosphorous tribromide (0.706 g, 2.6 mmol) drop wise over 10 min. The resulting reaction mixture was stirred at 0° C. for additional hour, which was followed by TLC. After completion of the reaction progress, it was quenched with water (5 mL), the diethyl ether was removed under a reduced pressure and the oily residue was diluted with water (30 mL). The aqueous material was then extracted with n-hexanes (3×50 mL), the combined hexanes were washed with brine (50 mL) dried over anhydrous MgSO₄ and concentrated under a reduced pressure to afford the desired trans-allylic bromide 10 (crude, 2.02 g, ˜90%). The bromide was dried under high vacuum and used in the next step without any additional purification to prepare ketoester 11.

### 3-Racemic ketoester 11

A reaction flask equipped with N₂ inlet, stir bar was charged with NaOEt (21% ethanolic solution, 3.24 mL, 10 mmol) followed by EtOH (5 mL). After cooling the reaction flask to 0° C., the addition of ethyl acetoacetate 3 (1.3 g, 10 mmol) was performed over 10 minutes and the resulting mixture was stirred at 30-45 min at the same temperature. To it at the same temperature was added bromide 10, (2.02 g, 7.14 mmol) as 1,4-dioxane (5 mL) solution over 10-15 minutes. The resulting reaction mixture was then allowed to attain at room temperature and stirred for overnight (˜16 h). The reaction progress was monitored by TLC. The reaction mixture was diluted with water (˜20 mL), and was extracted with n-hexanes (3×25 mL), dried over anhydrous Na₂SO₄ and the solvent was evaporated under a reduced pressure to afford a crude product containing keto ester 11 and unreacted/excess ethyl acetoacetate. The keto ester was purified by silica gel column chromatography using n-hexanes to 1-2% EtOAc in n-hexanes to afford a colorless racemic keto ester 11, TLC Rf: 0.41 (5% EtOAc/Hexanes); LCMS: MS (/z): 403 (MH+).

### 5E,9E,13E-Geranylgeranyl acetone 12 (5-trans-GGA)

A mixture of 3-rac-ketoester 11, (0.07 g, 0.174 mmol) MeOH (0.3 mL), and 5N aqueous KOH (0.15 mL) was heated at 80-85° C. for 2 h, reaction was followed by TLC. After cooling the reaction mixture, it was acidified with 2N HCl and extracted with diethyl ether, ethyl acetate or hexanes (3×400 mL). The combined organic layers were successively washed with water, aqueous NaHCO₃, brine and dried over anhydrous MgSO₄. After removal of solvent, the oily crude product was purified by silica gel column chromatography using n-hexanes to 1-2% EtOAc in n-Hexanes to afford a colorless liquid of 5-trans-GGA 12. Yield: 0.028 g (50%). TLC Rf: 0.45 (5% EtOAc/n-hexanes); LCMS: MS (m/z): 333 (MH+); 353 (M+Na).

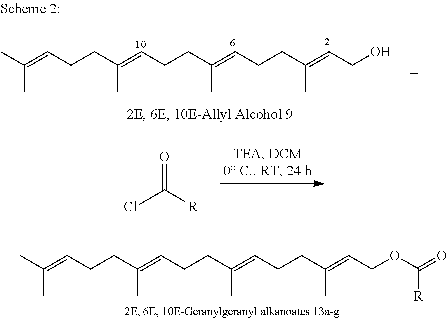

### 2E,6E,10E-Geranylgeranyl acetate 13a (R=Methyl)

A dry reaction flask equipped with a stir bar and N₂ inlet was charged with allyl alcohol 9 (0.087 g, 0.3 mmol), triethyl amine (0.062 mL, 0.45 mmol) and dichloromethane, DCM (1 mL) and cooled to 0° C. To it was added acetyl chloride (1M solution in DCM, 0.42 mL, 0.042 mmol) drop-wise and the resulting reaction was stirred at room temperature for overnight, ˜24 h. The reaction was quenched with aqueous NaHCO₃ solution, extracted with DCM (3×20 mL), the DCM extract was washed with water (20 mL), dried over anhydrous Na₂SO₄ and solvent was evaporated under a reduced pressure. The resulting oily residue was purified by a silica gel column chromatography using n-hexanes to 1-2% EtOAC in n-hexanes to afford a colorless liquid of ester 13a. Yield: 0.059 mg (60%); TLC Rf: 0.58 (10% EtOAc/n-Hexanes); LCMS: MS (m/z): 333.4 (MH+).

### 2E,6E,10E-Geranylgeranyl propionate 13b (R=Ethyl)

Similar to the preparation of ester 13a, the reaction of alcohol 9 with n-propionyl chloride afforded the desired compound 13b in 63% yield (0.065 g) as colorless oil. TLC Rf: 0.57 (10% EtOAc/n-hexanes); LCMS: MS (m/z): 347 (MH+).

### 2E,6E,10E-Geranylgeranyl iso-butyrate 13c (R=iso-Propyl)

Similar to the preparation of ester 13a, the reaction of alcohol 9 with iso-butyryl chloride afforded the desired compound 13c in 57% yield (0.061 g) as colorless oil. TLC Rf: 0.55 (10% EtOAc/n-hexanes); LCMS: MS (m/z): 361 (MH+).

### 2E,6E,10E-Geranylgeranyl cyclopropionate 13d (R=Cyclopropyl)

Similar to the preparation of ester 13a, the reaction of alcohol 9 with cyclopropanecarbonyl chloride gave the desired compound 13d in 54% yield (0.057 g) as colorless oil. TLC Rf: 0.54 (10% EtOAc/n-hexanes); LCMS: MS (m/z): 359 (MH+).

### 2E,6E,10E-Geranylgeranyl cyclopentanoate 13e (R=Cyclopentyl)

Similar to the preparation of ester 13a, the reaction of alcohol 9 with cyclopentanecarbonyl chloride gave the compound 13e in 61% yield (0.065 g) as colorless oil. TLC Rf: 0.53 (10% EtOAc/n-hexanes); LCMS: MS (m/z): 290 (M-Cyclopencarbonyl).

### 2E,6E,10E-Geranylgeranyl cyclohexanoate 13f (R=Cyclohexyl)

Similar to the preparation of ester 13a, the reaction of alcohol 9 with cyclohexanecarbonyl chloride gave the compound 13f in 65% yield (0.078 g) as colorless oil. TLC Rf: 0.53 (10% EtOAc/n-hexanes); LCMS: MS (m/z): 401 (MH+).

### 2E,6E,10E-Geranylgeranyl-3′,5′-dinitrobenzoate 13g (R=3′,5′=Dinitrophenyl)

Similar to the preparation of ester 13a, the reaction of alcohol 9 with 3,5-dinitrobenzoyl chloride gave the desired compound 13g in 60% yield (0.145 g) as colorless oil. TLC Rf: 0.46 (7% EtOAc/n-hexanes); LCMS: MS (m/z): 484.30 (M+).

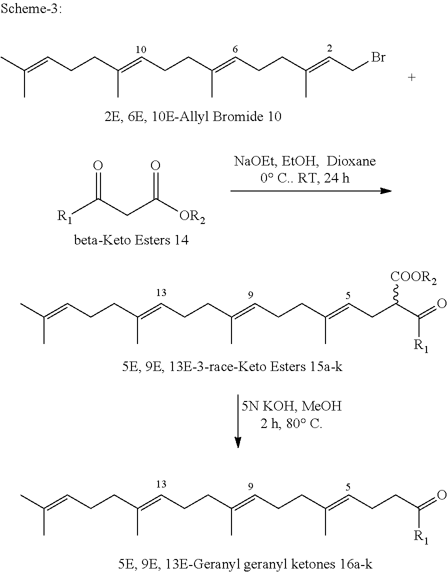

### 3-rac-Carboethoxy-5E,9E,13E-geranylgeranyl-1-n-propylacetone 15a (R₁═—CH₂CH₂CH₂CH₃; R₂═CH₂CH₃)

A dry reaction flask equipped with stir bar, N₂ inlet was charged with NaOEt (21% solution in EtOH, 0.226 mmol, 0.7 mmol), EtOH (0.5 mL). To it was added beta-ketoester 14 (R₁=n-butyl; R₂=Et; 0.110 g, 0.7 mmol) dropwise at 0° C., stirred for 30 minutes at 0° C. and another 30 minutes at room temperature. The reaction mixture was cooled to 0° C. and to it was added bromide 10 (0.166 g, 0.5 mmol) as a dioxane (0.5 mL) solution dropwise. The resulting reaction was stirred at room temperature for 24 h, quenched with water (10 mL), extracted with ethyl acetate (3×20 mL), the combined ethyl acetate extracts were dried over anhydrous Na₂SO₄ and solvent was evaporated under a reduced pressure. The obtained oily residue was then purified by silica gel column chromatography using n-hexanes then 1-2% EtOAc in n-hexanes to obtain the desired keto ester 15a. Yield: 0.133 g (60%); TLC Rf: 0.39 (5% EtOAc/n-hexanes); LCMS: MS (m/z): 445.50 (MH+).

### 5E,9E,13E-Geranylgeranyl-1-n-propyl acetone 16a (R₁═—CH₂CH₂CH₂CH₃)

A reaction flask containing keto ester 15a (0.088 g, 0.2 mmol), MeOH (0.5 mL), and 5N KOH (0.2 mL) was stirred at 80-90° C. for 2 h. Upon cooling the reaction at room temperature, it was diluted with water (10 mL), extracted with EtOAc (3×25 mL). The combined EtOAc extracts were dried and solvent was evaporated to obtain the oily material, which was purified by silica gel column chromatography using n-hexanes then 1-2% EtOAc in n-hexanes to obtain 0.029 g (40%) of the desired geranylgeranyl-1-n-propyl acetone 16a. TLC Rf: 0.42 (5% EtOAc/n-Hexanes); LCMS: MS (m/z): 373.60 (MH+).

### 3-rac-Carbomethoxy-5E,9E,13E-geranylgeranyl-1,1,1-trimethyl acetone 15b (R₁=tert-Butyl; R₂═CH₃)

Similar to the preparation of keto ester 15a, the reaction of bromide 10 with methyl 4,4-dimethyl-3-oxopentanoate afforded the requisite compound 15b, which was used in the next step without purification. TLC Rf: 0.37 (5% EtOAc/n-hexanes).

### 5E,9E,13E-Geranylgeranyl-1,1,1-trimethyl acetone 16b (R₁=tert-Butyl)

By using analogous procedure that was used to prepare 16a, the hydrolysis followed by decarboxylation of keto ester 15b (0.088 g, 0.2 mmol) afforded 0.051 g (71%) of 16b. TLC Rf: 0.40 (5% EtOAc/n-hexanes); LCMS: (m/z): 373.50 (MH+).

### 3-rac-Carbomethoxy-5E,9E,13E-geranylgeranyl-1-methyl acetone 15c (R₁═CH₂CH₃; R₂═CH₃)

By using analogous procedure that was used to prepare 15a, the reaction of bromide 10 with methyl propionylacetate gave 0.120 g (75%) of the desired 15c. TLC Rf: 0.35 (5% EtOAc/n-hexanes); LCMS: MS (m/z): 403.50 (MH+).

### 5E,9E,13E-geranylgeranyl-1-methyl acetone 16c (R₁═CH₂CH₃)

By using analogous procedure that of used for the preparation of 16a, the hydrolysis followed by decarboxylation of keto ester 15c (0.08 g, 0.2 mmol) afforded 0.041 g (59%) of 16c. TLC Rf: 0.38 (5% EtOAc/n-hexanes); LCMS: (m/z): 345.57 (MH+).

### 3-rac-Carboethoxy-5E,9E,13E-geranylgeranyl cyclopronanone 15d (R₁=cyclopropyl; R₂═CH₂CH₃)

By using analogous procedure that was used to prepare 15a, the reaction of bromide 10 with ethyl 3-cyclopropyl-3-oxopropanoate gave 0.111 g (50%) of the desired 15d. TLC Rf: 0.41 (5% EtOAc/n-hexanes); LCMS: MS (m/z): 429 (MH+).

### 5E,9E,13E-geranylgeranyl cyclopropanone 16d (R₁═cyclopropyl)

By using analogous procedure that of used for the preparation of 16a, the hydrolysis followed by decarboxylation of keto ester 15d (0.084 g, 0.2 mmol) afforded 0.040 g (57%) of 16d. TLC Rf: 0.52 (5% EtOAc/n-hexanes); LCMS: (m/z): 357.40 (MH+).

### 3-rac-Carboethoxy-1,1-dimethyl-5E,9E,13E-geranylgeranyl acetone 15e (R₁=iso-propyl; R₂═CH₂CH₃)

By using analogous procedure that was used to prepare 15a, the reaction of bromide 10 with ethyl 4-methyl-3-oxopentanoate gave 0.043 g (20%) of the desired 15e. TLC Rf: 0.56 (10% EtOAc/n-hexanes); LCMS: MS (m/z): 431.50 (MH+).

### 1,1-Dimethyl-5E,9E,13E-geranylgeranyl acetone 16e (R₁=iso-propyl)

By using analogous procedure that of used for the preparation of 16a, the hydrolysis followed by decarboxylation of keto ester 15e (0.084 g, 0.2 mmol) afforded 0.032 g (45%) of 16e. TLC Rf: 0.66 (10% EtOAc/n-hexanes); LCMS: (m/z): 359.60 (MH+).

### 3-rac-Carboethoxy-1-ethyl-5E,9E,13E-geranylgeranyl acetone 15f (R₁═CH₂CH₂CH₃; R₂═CH₂CH₃)

By using analogous procedure that was used to prepare 15a, the reaction of bromide 10 with ethyl 3-oxohexanoate gave 0.129 g (60%) of the desired 15f. TLC Rf: 0.64 (10% EtOAc/n-hexanes); LCMS: MS (m/z): 431.50 (MH+).

### 1-Ethyl-5E,9E,13E-geranylgeranyl acetone 16f (R₁═CH₂CH₂CH₃)

By using analogous procedure that of used for the preparation of 16a, the hydrolysis followed by decarboxylation of keto ester 15f (0.086 g, 0.2 mmol) afforded 0.041 g (57%) of 16f. TLC Rf: 0.56 (5-7% EtOAc/n-hexanes); LCMS: (m/z): 359.50 (MH+).

### 1-Adamentyl-3-rac-Carboethoxy-5E,9E,13E-geranylgeranyl ketone 15g (R₁=1-Adamentyl; R₂═CH₂CH₃)

By using analogous procedure that was used to prepare 15a, the reaction of bromide 10 with ethyl 3-(1-adamantyl)-3-oxopropanoate gave 0.154 g (59%) of the desired 15g. TLC Rf: 0.58 (10% EtOAc/n-hexanes); LCMS: MS (m/z): 523.30 (MH+).

### 1-Adamentyl-5E,9E,13E-geranylgeranyl ketone 16g (R₁=1-Adamentyl)

By using analogous procedure to that was used for the preparation of 16a, the hydrolysis followed by decarboxylation of keto ester 15g (0.104 g, 0.2 mmol) afforded 0.042 g (46%) of 16g. TLC Rf: 0.63 (10% EtOAc/n-hexanes); LCMS: (m/z): 451 (MH+).

### 3-rac-Carbomethoxy-5E,9E,13E-geranylgeranyl-1-methoxy acetone 15h (R₁═CH₂—O—CH₃; R₂═CH₃)

By using analogous procedure that was used to prepare 15a, the reaction of bromide 10 with methyl 4-methoxy-3-oxobutanoate gave 0.062 g (30%) of the desired 15h. TLC Rf: 0.20 (10% EtOAc/n-hexanes); LCMS (m/z): 419.30 (MH+).

### 5E,9E,13E-geranylgeranyl-1-methoxy acetone 16h (R₁═CH₂—O—CH₃)

By using analogous procedure to that was used for the preparation of 16a, the hydrolysis followed by decarboxylation of keto ester 15h (0.060 g, 0.15 mmol) afforded 0.017 g (31%) of 16h. TLC Rf: 0.28 (10% EtOAc/n-hexanes); LCMS: (m/z): 361.30 (MH+).

### 3-rac-Carbomethoxy-5E,9E,13E-geranylgeranyl-1-methylenemethoxy acetone 15i (R₁═CH₂CH₂—O—CH₃; R₂═CH₃)

By using analogous procedure that was used to prepare 15a, the reaction of bromide 10 with methyl 5-methoxy-3-oxopentanoate gave 0.052 g (24%) of the desired 15i. TLC Rf: 0.20 (10% EtOAc/n-hexanes), LCMS (m/z): 419.30 (MH+).

### 5E,9E,13E-Geranylgeranyl-1-methylenemethoxy acetone 16i (R₁═CH₂CH₂—O—CH₃)

By using analogous procedure to that was used for the preparation of 16a, the hydrolysis followed by decarboxylation of keto ester 15i (0.05 g, 0.11 mmol) afforded 0.008 g (19%) of 16i. TLC Rf: 0.27 (10% EtOAc/n-hexanes); LCMS: (m/z): 375 (MH+).

### 1-Allyl-3-rac-Carbomethoxy-5E,9E,13E-geranylgeranyl acetone 15j (R₁═CH₂CH₂CH═CH₂; R₂═CH₂CH₃)

By using analogous procedure that was used to prepare 15a, the reaction of bromide 10 with methyl 3-oxo-6-heptenoate gave 0.089 g (42%) of the desired 15j. TLC Rf: 0.60 (10% EtOAc/n-hexanes); LCMS: MS (m/z): 429.60 (MH+).

### 1-Allyl-5E,9E,13E-geranylgeranyl acetone 16j (R₁═CH₂CH₂CH═CH₂)

By using analogous procedure to that was used for the preparation of 16a, the hydrolysis followed by decarboxylation of keto ester 15j (0.084 g, 0.2 mmol) afforded 0.022 g (31%) of 16j. TLC Rf: 0.71 (10% EtOAc/n-hexanes); LCMS: (m/z): 371.60 (MH+).

### 1-Fur-3′-yl-3-rac-carboethoxy-5E,9E,13E-geranylgeranyl acetone 15k (R₁=3-Furanyl; R₂═CH₂CH₃)

By using analogous procedure that was used to prepare 15a, the reaction of bromide 10 with ethyl 3-(3-furyl)-3-oxopropanoate gave 0.065 g (29%) of the desired 15k was prepared using analogous procedure that was used to prepare of keto ester 15a. TLC Rf: 0.48 (10% EtOAc/n-hexanes); LCMS: MS (m/z): 455.30 (MH+).

### 1-Fur-3′-yl-5E,9E,13E-geranylgeranyl acetone 16k (R₁=3-Furanyl)

By using analogous procedure to that was used for the preparation of 16a, the hydrolysis followed by decarboxylation of keto ester 15k (0.06 g, 0.13 mmol) afforded 0.014 g (26%) of 16k. TLC Rf: 0.62 (10% EtOAc/n-hexanes); LCMS: (m/z): 383.60 (MH+).

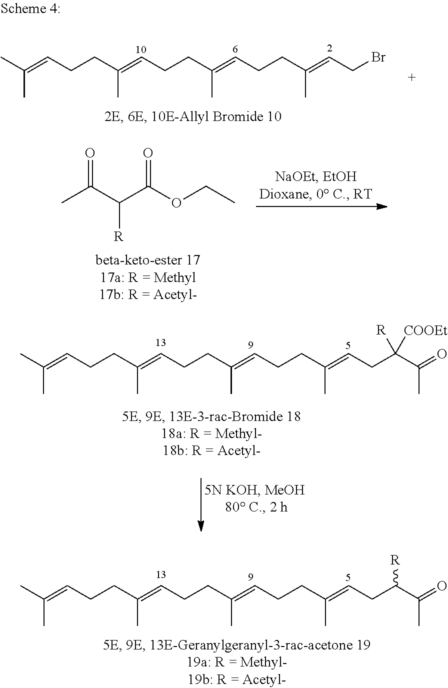

### 5E,9E,13E-Geranyl geranyl-rac-3-methyl-3-carboethoxy acetone 18a

A dry reaction flask equipped with stir bar, N₂ inlet was charged with NaOEt (21% solution in EtOH, 0.226 mmol, 0.7 mmol), EtOH (0.5 mL). To it was added beta-ketoester 17a (0.100 g, 0.7 mmol) dropwise at 0° C., stirred for 30 minutes at 0° C. and another 30 minutes at room temperature. The reaction mixture was cooled to 0° C. and to it was added bromide 10 (0.166 g, 0.5 mmol) as a dioxane (0.5 mL) solution dropwise. The resulting reaction was stirred at room temperature for 24 h, quenched with water (10 mL), extracted with ethyl acetate (3×20 mL), the combined ethyl acetate extracts were dried over anhydrous Na₂SO₄ and solvent was evaporated under a reduced pressure. The obtained oily residue was then purified by silica gel column chromatography using n-hexanes then 1-2% EtOAc in n-hexanes to obtain the desired 3-rac-keto ester 18a. Yield: 0.100 g (48%); TLC Rf: 0.47 (10% EtOAc/n-hexanes); LCMS: MS (m/z): 417.50 (MH+).

### 5E,9E,13E-Geranyl geranyl-rac-3-methyl-3-carboethoxy acetone 18b

Similar to the preparation of ketoester 18a, the ketoester 18b was prepared. Yield: 0.133 g (60%); TLC Rf: 0.40 (10% EtOAc/n-hexanes).

### 5E,9E,13E-Geranyl geranyl-rac-3-methyl acetone 19a

A reaction flask containing keto ester 18a (0.083 g, 0.2 mmol), MeOH (0.5 mL), and 5N KOH (0.2 mL) was stirred at 80-90° C. for 2 h. Upon cooling the reaction at room temperature, it was diluted with water (10 mL), extracted with EtOAc (3×25 mL). The combined EtOAc extracts were dried and solvent was evaporated to obtain the oily material, which was purified by silica gel column chromatography using n-hexanes then 1-2% EtOAc in n-hexanes to obtain 0.057 g (84%) of the desired geranyl geranyl-rac-3-acetyl acetone 19b. TLC Rf: 0.33 (10% EtOAc/n-Hexanes); LCMS: MS (m/z): 345.60 (MH+).

### 5E,9E,13E-Geranyl geranyl-rac-3-acetyl acetone 19b

Similar to the preparation of ketone 19a, the diacetyl compound 19b was prepared. Yield: 0.61 g (55%); TLC Rf: 0.43 (10% EtOAc/n-hexanes); LCMS: MS (m/e) 373 (MH+).

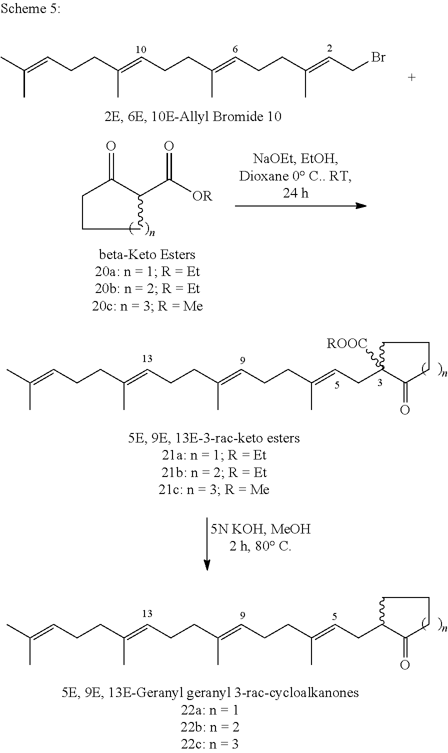

### rac-3-Carboethoxy-5E,9E,13E-geranylgeranyl cyclopentanone 21a

A dry reaction flask equipped with stir bar, N₂ inlet was charged with NaOEt (21% solution in EtOH, 0.226 mmol, 0.7 mmol), EtOH (0.5 mL). To it was added beta-ketoester 20a (0.100 mL, 0.7 mmol) dropwise at 0° C., stirred for 30 minutes at 0° C. and another 30 minutes at room temperature. The reaction mixture was cooled to 0° C. and to it was added bromide 10 (0.166 g, 0.5 mmol) as a dioxane (0.5 mL) solution dropwise. The resulting reaction was stirred at room temperature for 24 h, quenched with water (10 mL), extracted with ethyl acetate (3×20 mL), the combined ethyl acetate extracts were dried over anhydrous Na₂SO₄ and solvent was evaporated under a reduced pressure. The obtained oily residue was then purified by silica gel column chromatography using n-hexanes then 1-2% EtOAc in n-hexanes to obtain the desired keto ester 21a. Yield: 0.098 g (46%); TLC Rf: 0.40 (10% EtOAc/n-hexanes); LCMS: MS (m/z): 429.40 (MH+).

### 5E,9E,13E-Geranylgeranyl-rac-3-cyclopentanone 22a

A reaction flask containing keto ester 21a (0.086 g, 0.2 mmol), MeOH (0.5 mL), and 5N KOH (0.2 mL) was stirred at 80-90° C. for 2 h. Upon cooling the reaction at room temperature, it was diluted with water (10 mL), extracted with EtOAc (3×25 mL). The combined EtOAc extracts were dried and solvent was evaporated to obtain the oily material, which was purified by silica gel column chromatography using n-hexanes then 1-2% EtOAc in n-hexanes to obtain 0.036 g (51%) of the desired geranyl geranyl-rac-3-cyclopentanone 22a. TLC Rf: 0.41 (10% EtOAc/n-Hexanes); LCMS: MS (m/z): 357.40 (MH+).

### rac-3-carboethoxy-5E,9E,13E-Geranylgeranyl cyclohexanone 21b

A dry reaction flask equipped with stir bar, N₂ inlet was charged with NaOEt (21% solution in EtOH, 0.226 mmol, 0.7 mmol), EtOH (0.5 mL). To it was added beta-ketoester 20b (0.112 mL, 0.7 mmol) dropwise at 0° C., stirred for 30 minutes at 0° C. and another 30 minutes at room temperature. The reaction mixture was cooled to 0° C. and to it was added bromide 10 (0.166 g, 0.5 mmol) as a dioxane (0.5 mL) solution dropwise. The resulting reaction was stirred at room temperature for 24 h, quenched with water (10 mL), extracted with ethyl acetate (3×20 mL), the combined ethyl acetate extracts were dried over anhydrous Na₂SO₄ and solvent was evaporated under a reduced pressure. The obtained oily residue was then purified by silica gel column chromatography using n-hexanes then 1-2% EtOAc in n-hexanes to obtain the desired keto ester 21b. Yield: 0.128 g (58%); TLC Rf: 0.45 (10% EtOAc/n-hexanes); LCMS: MS (m/z): 443.50 (MH+).

### 5E,9E,13E-Geranyl geranyl-rac-3-cyclohexanone 22b

A reaction flask containing keto ester 21b (0.088 g, 0.2 mmol), MeOH (0.5 mL), and 5N KOH (0.2 mL) was stirred at 80-90° C. for 2 h. Upon cooling the reaction at room temperature, it was diluted with water (10 mL), extracted with EtOAc (3×25 mL). The combined EtOAc extracts were dried and solvent was evaporated to obtain the oily material, which was purified by silica gel column chromatography using n-hexanes then 1-2% EtOAc in n-hexanes to obtain 0.039 g (53%) of the desired geranylgeranyl-rac-3-cyclohexanone 22b. TLC Rf: 0.47 (10% EtOAc/n-Hexanes); LCMS: MS (m/z): 411 (M+ acetonitrile).

### rac-Carbomethoxy-5E,9E,13E-geranylgeranyl cycloheptanone 21c

A dry reaction flask equipped with stir bar, N₂ inlet was charged with NaOEt (21% solution in EtOH, 0.226 mmol, 0.7 mmol), EtOH (0.5 mL). To it was added beta-ketoester 20c (0.112 mL, 0.7 mmol) dropwise at 0° C., stirred for 30 minutes at 0° C. and another 30 minutes at room temperature. The reaction mixture was cooled to 0° C. and to it was added bromide 10 (0.166 g, 0.5 mmol) as a dioxane (0.5 mL) solution dropwise. The resulting reaction was stirred at room temperature for 24 h, quenched with water (10 mL), extracted with ethyl acetate (3×20 mL), the combined ethyl acetate extracts were dried over anhydrous Na₂SO₄ and solvent was evaporated under a reduced pressure. The obtained oily residue was then purified by silica gel column chromatography using n-hexanes then 1-2% EtOAc in n-hexanes to obtain the desired keto ester 21c. Yield: 0.125 g (55%); TLC Rf: 0.42 (10% EtOAc/n-hexanes); LCMS: MS (m/z): 457.50 (MH+).

### 5E,9E,13E-Geranyl geranyl-rac-3-cycloheptanone 22c

A reaction flask containing keto ester 21c (0.092 g, 0.2 mmol), MeOH (0.5 mL), and 5N KOH (0.2 mL) was stirred at 80-90° C. for 2 h. Upon cooling the reaction at room temperature, it was diluted with water (10 mL), extracted with EtOAc (3×25 mL). The combined EtOAc extracts were dried and solvent was evaporated to obtain the oily material, which was purified by silica gel column chromatography using n-hexanes then 1-2% EtOAc in n-hexanes to obtain 0.037 g (49%) of the desired geranyl geranyl-rac-3-cycloheptanone 22c. TLC Rf: 0.44 (10% EtOAc/n-Hexanes); LCMS: MS (m/z): 425 (M+ acetonitrile).

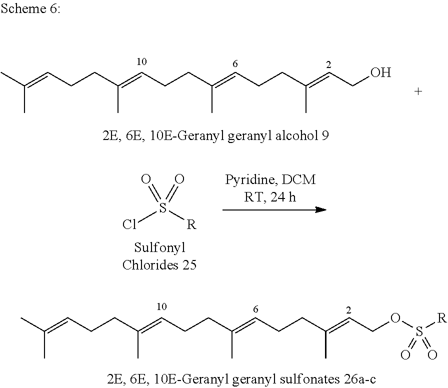

### 2E,6E,10E-Geranylgeranyl methanesulfonate 26a (R=Methyl-)

A dry reaction flask equipped with a stir bar and N₂ inlet was charged with geranylgeranyl alcohol 9 (0.087 g, 0.3 mmol), pyridine (0.048 mL, 0.6 mmol) in DCM (2 mL). To it was added, methanesulfonyl chloride 25a (0.035 mL, 0.45 mmol) and stirred for 48 h at room temperature. The reaction was followed by TLC. After the completion of the reaction, it was quenched with water (10 mL), extracted with DCM (3×20 mL) and the combined DCM solution was washed with 2N NaOH solution (20 mL) followed by water (20 mL). The DCM layer upon drying over anhydrous Na₂SO₄ was evaporated and the residue was purified by silica gel column chromatography using n-hexanes the 1-2% EtOAc in n-hexanes to afford the desired sulfonate 26a. Yield: 0.066 g (66%); TLC Rf: 0.54 (10% EtOAc/n-Hexanes); LCMS: MS (m/z): 367.10 (M-H).

The following sulfonates 26b and 26c were prepared according to the procedure used to prepare sulfonate 26a.

### 2E,6E,10E-Geranylgeranyl benzenesulfonate (26b; R=Phenyl)

The reaction of alcohol 9 with benzenesulfonyl chloride afforded the requisite sulfonate 26b. Yield: 0.087 g (68%); TLC Rf: 0.45 (10% EtOAc/n-Hexanes); LCMS: MS (m/z): 471.30 (M+Acetonitrile).

### 2E,6E,10E-Geranylgeranyl p-toluenesulfonate (26c; R=p-Toluene)

The reaction of alcohol 9 with p-toluenesulfonyl chloride afforded the requisite sulfonate 26c. Yield: 0.072 g (54%); TLC Rf: 0.42 (10% EtOAc/n-Hexanes); LCMS: MS (m/z): 443.50 (M-H).

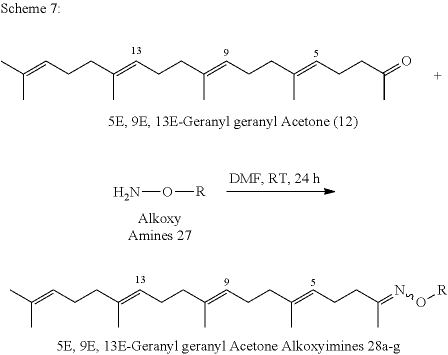

### 2E,6E,10E-Geranylgeranyl acetone hydroxyimine (28a; R═H)

To a dry reaction flask was placed Geranylgeranyl acetone 12 (0.066 g, 0.2 mmol) and dimethylformamide (DMF) (0.5 mL) under N2. To it was added hydroxylamine.HCl 27a (0.012 g, 0.3 mmol) and stirred for overnight at room temperature. The reaction was followed by TLC. After the reaction was completed, it was quenched with water (10 mL) and extracted with n-hexanes (2×20 mL). The n-hexanes layers were combined, washed with water (10 mL), dried over anhydrous Na₂SO₄ and solvent was evaporated. The resulting product was single spot by TLC so it was dried under high vacuum to obtain 0.020 g (28%) of hydroxyimine 28a. TLC Rf: 0.23 (5% EtOAc/Hexanes); LCMS: MS (m/z): 346.30 (MH+).

By using making use of the procedure used to prepare hydroxyimine 28a, the following alkoxyimines 28b to 28g were prepared by reacting ketone 12 with the corresponding alkoxy amines 27 and purified by silica gel column chromatography using n-hexanes to 1-2% EtOAc in n-hexanes.

### 2E,6E,10E-Geranylgeranyl acetone methoxyimine (28b; R=Methyl)

The reaction of ketone 12 with methyloxy amine afforded the corresponding methyloxyimine 28b. Yield: 0.038 g (53%). TLC Rf: 0.69 (5% EtOAc/Hexanes); LCMS: MS (m/z): 360 (MH+).

### 2E,6E,10E-Geranylgeranyl acetone ethoxyimine (28c; R=Ethyl)

The reaction of ketone 12 with ethyloxy amine afforded the corresponding ethyloxyimine 28c. Yield: 0.057 g (51%). TLC Rf: 0.5 (5% EtOAc/Hexanes); LCMS: MS (m/z): 374.40 (MH+).

### 2E,6E,10E-Geranylgeranyl acetone allyloxyimine (28d; R=Allyl)

The reaction of ketone 12 with allyloxy amine afforded the corresponding allyloxyimine 28d Yield: 0.055 g (48%). TLC Rf: 0.5 (5% EtOAc/Hexanes); LCMS: MS (m/z): 386.40 (MH+).

### 2E,6E,10E-Geranylgeranyl acetone tetrahydro-2H-pyran-2-oxyimine (28e; R=Tetrahydro-2H-pyran)

The reaction of ketone 12 with tetrahydro-2H-pyran-2-oxyamine afforded the corresponding tetyrahydro-2H-pyran-2-oxyimine 28e. Yield: 0.039 g (30%). TLC Rf: 0.2 (5% EtOAc/Hexanes); LCMS: MS (m/z): 346.40 (M-THP).

### 2E,6E,10E-Geranylgeranyl acetone benzyloxyimine (28f; R=Benzyl)

The reaction of ketone 12 with benzyloxy amine afforded the corresponding benzyloxyimine 28f. Yield: 0.060 g (46%). TLC Rf: 0.45 (5% EtOAc/Hexanes); LCMS: MS (m/z): 436.40 (MH+).

### 2E,6E,10E-Geranylgeranyl acetone carboxymethyloxyimine (28g; R=Carboxymethyl)

The reaction of ketone 12 with carbomethyloxy amine afforded the corresponding carbomethyloxyimine 28g. Yield: 0.073 g (61%). TLC Rf: 0.2 (5% EtOAc/Hexanes); LCMS: MS (m/z): 404.40 (MH+).

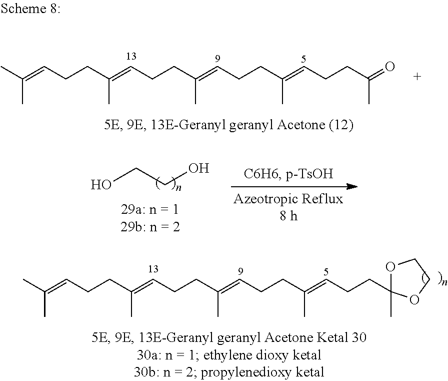

### 5E,9E,13E-Geranylgeranyl acetone 2,2-ethylenedioxyketal 30a

A dry reaction flask equipped with a stir bar, azeotropic reflux unit was charged with ketone 12 (0.110 g, 0.333 mmol), ethyelene glycol 29a (0.103 g, 1.66 mmol), p-TsOH (10 mg) and benzene (15 mL) and refluxed azeotropically for 8 hours to remove the liberated water. The resulting reaction mixture was quenched with aqueous NaHCO₃ solution and washed with water. The organic layer upon drying over anhydrous Na₂SO₄ was concentrated under a reduced pressure to afford a pure ketal 30a. TLC Rf: 0.30 (5% EtOAc/n-hexanes); Yield 0.112 g (90%); LCMS: MS (m/e) 331 (M− —CH₂CH₂OH).

### 5E,9E,13E-Geranylgeranyl acetone 2,2-(1,3-propelyenedioxy)-ketal 30b

Similar to the preparation of ketal 30a, the ketal 30b was prepared from the reaction of ketone 12 and 1,3-propelyne glycol 29b. Yield: 0.119 g (60%); TLC Rf: 0.30 (5% EtOAc/n-hexanes); LCMS: MS (m/z): 389.40 (MH+), 331 (M− —CH₂CH₂CH₂OH).

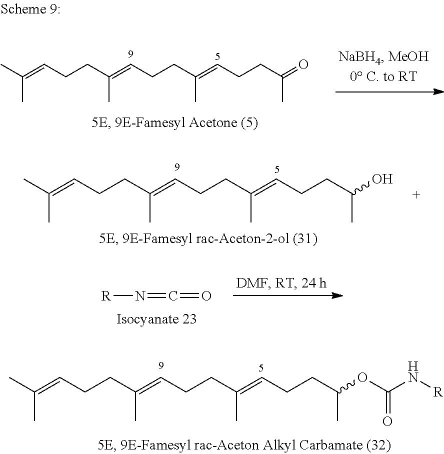

### 5E,9E-Farnesyl rac-acet-2-ol (31)

A reaction flask with a stir bar and N₂ inlet was charged with ketone 5 (1.2 g, 5 mmol) and MeOH (10 mL). After cooling the reaction flask to 0° C., the addition of NaBH₄ (0.190 g, 5 mmol) was performed in portions over several minutes and the reaction was stirred for additional hour. The reaction was monitored by TLC. The reaction was quenched with H₂O (40 mL) and the product was extracted with EtOAc (3×50 mL), dried over anhydrous Na₂SO₄ and solvent was removed under a reduced pressure to obtain the desired alcohol 31. Yield: 1.25 g (95%); TLC Rf: 0.24 (10% EtOAc/n-hexanes); LCMS: MS (m/z): 265 (MH+).

### Ethyl 5E,9E-farnesyl rac-prop-2-yl carbamate 32a (R=Ethyl)

A dry reaction flask equipped with a stir bar, N₂ inlet was charged with alcohol 31 (0.052 g, 0.2 mmol), pyridine (0.032 mL, 0.4 mmol) and DCM (2 mL). After cooling it to 0° C., ethyl isocyanate was added dropwise and the resulting reaction mixture was allowed to stir for 24 h. The reaction was monitored by TLC. After completion of the reaction, it was quenched with H₂O (5 mL), acidified, extracted with n-hexanes (3×15 mL) and the combined n-hexanes were washed with H₂O (10 mL). After drying the organic solution over anhydrous Na₂SO₄, the solvent was evaporated and the resulting residue was purified by silica gel column chromatography using 1-2% EtOAc in n-hexanes to afford the desired carbamate 32a. Yield: 0.037 g (52%); TLC Rf: 0.23 (5% EtOAc/n-Hexanes); LCMS: MS (m/z): 336.40 (MH+).

The following carbamates 32b to 32j were prepared according to the procedure that was used to prepare carbamate 32a.

### iso-Butyryl 5E,9E-farnesyl rac-prop-2-yl carbamate 32b (R=iso-Butyryl)

The reaction of alcohol 31 with iso-butyryl isocyanate afforded the expected carbamate 32b. Yield: 0.038 g (50%); TLC Rf: 0.43 (10% EtOAc/n-Hexanes); LCMS: MS (m/z): 364 (MH+).

### iso-Propyl 5E,9E-farnesyl rac-prop-2-yl carbamate 32c (R=iso-Propyl-)

The reaction of alcohol 31 with iso-propyl isocyanate afforded the expected carbamate 32c. Yield: 0.036 g (48%); TLC Rf: 0.41 (10% EtOAc/n-Hexanes); LCMS: MS (m/z): 350.40 (MH+).

### n-Pentyl 5E,9E-farnesyl rac-prop-2-yl carbamate 32d (R=n-Pentyl)

The reaction of alcohol 31 with n-pentyl isocyanate afforded the expected carbamate 32d. Yield: 0.043 g (54%); TLC Rf: 0.40 (10% EtOAc/n-Hexanes); LCMS: MS (m/z): 378 (MH+).

### n-Hexyl 5E,9E-farnesyl rac-prop-2-yl carbamate 32e (R=n-Hexyl)

The reaction of alcohol 31 with n-hexyl isocyanate afforded the expected carbamate 32e. Yield: 0.040 g (49%); TLC Rf: 0.41 (10% EtOAc/n-Hexanes); LCMS: MS (m/z): 392 (MH+).

### Cyclopentyl 5E,9E-farnesyl rac-prop-2-yl carbamate 32f (R=Cyclopentyl)

The reaction of alcohol 31 with cyclopentyl isocyanate afforded the expected carbamate 32f. Yield: 0.035 g (45%); TLC Rf: 0.36 (10% EtOAc/n-Hexanes); LCMS: MS (m/z): 376.40 (MH+).

### Cyclohexyl 5E,9E-farnesyl rac-prop-2-yl carbamate 32g (R=Cyclohexyl)

The reaction of alcohol 31 with cyclohexyl isocyanate afforded the expected carbamate 32g. Yield: 0.040 g (54%); TLC Rf: 0.40 (10% EtOAc/n-Hexanes); LCMS: MS (m/z): 390.60 (MH+).

### Cyclohexylmethyl 5E,9E-farnesyl rac-prop-2-yl carbamate 32h (R=Cyclohexylmethyl)

The reaction of alcohol 31 with cyclohexylmethyl isocyanate afforded the expected carbamate 32h. Yield: 0.037 g (47%); TLC Rf: 0.40 (10% EtOAc/n-Hexanes); LCMS: MS (m/z): 404.60 (MH+).

### Cycloheptyl 5E,9E-farnesyl rac-prop-2-yl carbamate 32i (R=Cycloheptyl)

The reaction of alcohol 31 with cycloheptyl isocyanate afforded the expected carbamate 32i. Yield: 0.043 g (54%); TLC Rf: 0.54 (10% EtOAc/n-Hexanes); LCMS: MS (m/z): 404.60 (MH+).

### 5E,9E-farnesyl rac-prop-2-yl Methyl 2-(S)-(−)-3-methylbutyrate Carbamate 32j (R=Methyl-2-(S)-(−)-3-methylbutyrate)

The reaction of alcohol 31 with methyl 2-(S)-(−)-3-methylbutyryl isocyanate afforded the expected carbamate 32j. Yield: 0.41 g (49%); TLC Rf: 0.28 (10% EtOAc/n-Hexanes); LCMS: MS (m/z): 422.60 (MH+).

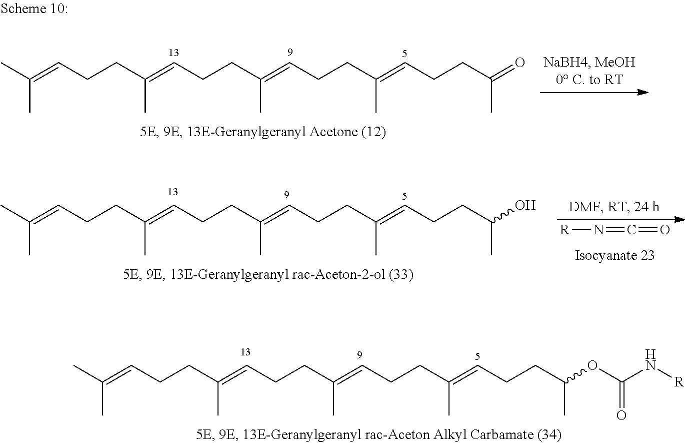

### 5E,9E,13E Geranylgeranyl aceton-2-ol (33)

A reaction flask with a stir bar and N₂ inlet was charged with ketone 12 (1.66 g, 5 mmol) and MeOH (10 mL). After cooling the reaction flask to 0° C., the addition of NaBH₄ (0.190 g, 5 mmol) was performed in portions over several minutes and the reaction was stirred for additional hour. The reaction was monitored by TLC. The reaction was quenched with H₂O (40 mL) and the product was extracted with EtOAc (3×50 mL), dried over anhydrous Na₂SO₄ and solvent was removed under a reduced pressure to obtain the desired alcohol 33. Yield: 1.53 g (92%); TLC Rf: 0.23 (10% EtOAc/n-hexanes); LCMS: MS (m/z): 335 (MH+).

### 5E,9E,13E-Geranylgeranyl-rac-prop-2-yl iso-propyl carbamate (34a; R=iso-Propyl)

A dry reaction flask equipped with a stir bar, N₂ inlet was charged with alcohol 33 (0.052 g, 0.2 mmol), pyridine (0.032 mL, 0.4 mmol) and DCM (2 mL). After cooling it to 0° C., iso-propyl isocyanate (0.49 mL, 0.5 mmol) was added dropwise and the resulting reaction mixture was allowed to stir for 24 h. The reaction was monitored by TLC. After completion of the reaction, it was quenched with H₂O (5 mL), acidified, extracted with n-hexanes (3×15 mL) and the combined n-hexanes were washed with H₂O (10 mL). After drying the organic solution over anhydrous Na₂SO₄, the solvent was evaporated and the resulting residue was purified by silica gel column chromatography using 1-2% EtOAc in n-hexanes to afford the desired carbamate 34a. Yield: 0.037 g (52%); TLC Rf: 0.23 (5% EtOAc/n-Hexanes); LCMS: MS (m/z): 336.40 (MH+).

The following carbamates 34b to 34g were prepared according to the procedure that was used to prepare carbamate 32a.

### 5E,9E,13E-Geranylgeranyl-rac-prop-2-yl n-pentyl carbamate (34b; R=n-Pentyl)

The reaction of alcohol 33 with n-pentyl isocyanate afforded the desired carbamate 34b. Yield: 0.040 g (46%); TLC Rf: 0.33 (10% EtOAc/n-Hexanes); LCMS: MS (m/z): 446.60 (MH+).

### Cyclopentyl 5E,9E,13E-geranylgeranyl-rac-prop-2-yl carbamate (34c; R=cyclopentyl)

The reaction of alcohol 33 with cyclopentyl isocyanate afforded the desired carbamate 34c. Yield: 0.041 g (47%); TLC Rf: 0.39 (10% EtOAc/n-Hexanes); LCMS: MS (m/z): 444.60 (MH+).

### Cyclohexylmethyl 5E,9E,13E-geranylgeranyl-rac-prop-2-yl carbamate (34d; R=cyclohexylmethyl)

The reaction of alcohol 33 with n-cyclohexylmethyl isocyanate afforded the desired carbamate 34d. Yield: 0.045 g (48%); TLC Rf: 0.25 (10% EtOAc/n-Hexanes); LCMS: MS (m/z): 472.60 (MH+).

### Cycloheptyl 5E,9E,13E-geranylgeranyl-rac-prop-2-yl carbamate (34e; R=cycloheptyl)

The reaction of alcohol 33 with cycloheptyl isocyanate afforded the desired carbamate 34e. Yield: 0.048 g (51%); TLC Rf: 0.57 (10% EtOAc/n-Hexanes); LCMS: MS (m/z): 472.40 (MH+).

### 5E,9E,13E-Geranylgeranyl-rac-prop-2-yl n-hexyl carbamate (34f; R=n-Hexyl)

The reaction of alcohol 33 with n-hexyl isocyanate afforded the desired carbamate 34f. Yield: 0.039 g (44%); TLC Rf: 0.36 (10% EtOAc/n-Hexanes); LCMS: MS (m/z): 446.40 (MH+).

### 5E,9E,13E-Geranylgeranyl-rac-prop-2-yl methyl 2-(S)-(−)-3-methylbutyryl carbamate (34g; R=Methyl 2-(S)-(−)-3-methylbutyrate)

The reaction of alcohol 33 with methyl 2-(S)-(−)-3-methylbutyryl isocyanate afforded the desired carbamate 34g. Yield: 0.049 g (51%); TLC Rf: 0.37 (10% EtOAc/n-Hexanes); LCMS: MS (m/z): 490.60 (MH+).

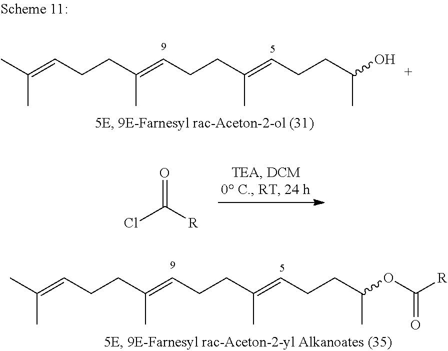

### 5E,9E-Farnesyl-rac-prop-2-yl acetate (35a; R=Methyl)

A dry reaction flask equipped with a stir bar and N₂ inlet was charged with alcohol 31 (0.053 g, 0.2 mmol), triethyl amine (0.04 mL, 0.3 mmol) and dichloromethane, DCM (2 mL) and cooled to 0° C. To it was added acetyl chloride (1M solution in DCM, 0.25 mL, 0.025 mmol) drop-wise and the resulting reaction was stirred at room temperature for overnight, ˜24 h. The reaction was quenched with aqueous NaHCO₃ solution, extracted with DCM (3×20 mL), the DCM extract was washed with water (20 mL), dried over anhydrous Na₂SO₄ and solvent was evaporated under a reduced pressure. The resulting oily residue was purified by a silica gel column chromatography using n-hexanes to 1-2% EtOAC in n-hexanes to afford a colorless liquid of ester 35a. Yield: 0.029 mg (49%); TLC Rf: 0.79 (5% EtOAc/n-Hexanes); LCMS: MS (m/z): 307.4 (MH+).

### 5E,9E-Farnesyl-rac-prop-2-yl propionate 35b (R=Ethyl)

Similar to the preparation of ester 35a, the reaction of alcohol 31 with propionyl chloride gave the ester 35b. Yield: 0.024 g (38%) as colorless oil. TLC Rf: 0.74 (5% EtOAc/n-hexanes).

### 5E,9E-Farnesyl-rac-prop-2-yl iso-butyrate 35c (R=iso-Propyl)

Similar to the preparation of ester 35a, the reaction of alcohol 31 with iso-butyryl chloride gave the ester 35c. Yield: 0.027 g (41%). TLC Rf: 0.78 (5% EtOAc/n-hexanes).

### 5E,9E-Farnesyl-rac-prop-2-yl cyclopropionate 35d (R=Cyclopropyl)

Similar to the preparation of ester 35a, the reaction of alcohol 31 with cyclopropionyl chloride gave the ester 35d. Yield: 0.023 g (35%). TLC Rf: 0.76 (5% EtOAc/n-hexanes).

### 5E,9E-Farnesyl-rac-prop-2-yl cyclopentanoate 35e (R=Cyclopentyl)

Similar to the preparation of ester 35a, the reaction of alcohol 31 with cyclopentanoyl chloride gave the ester 35e. Yield: 0.027 g (38%). TLC Rf: 0.86 (5% EtOAc/n-hexanes).

### 5E,9E-Farnesyl-rac-prop-2-yl cyclohexanoate 35f (R=Cyclohexyl)

Similar to the preparation of ester 35a, the reaction of alcohol 31 with cyclohexanoyl chloride gave the ester 35f. Yield: 0.027 g (37%). TLC Rf: 0.88 (5% EtOAc/n-hexanes).

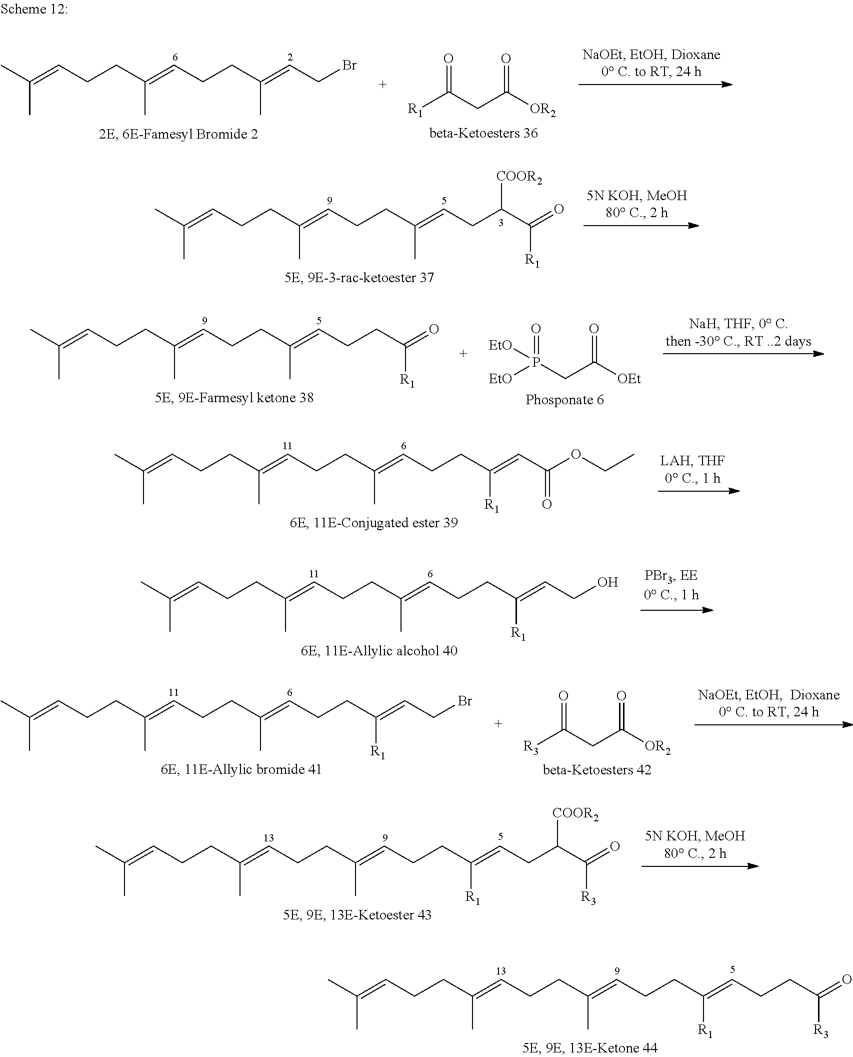

### rac-3-Carbomethoxy-5E,9E-farnesyl-1-methyl acetone 37a (R₁=Ethyl; R₂=Methyl)

A reaction flask equipped with N₂ inlet, stir bar was charged with NaOEt (21% ethanolic solution, 3.36 mL, 10.4 mmol) followed by EtOH (5 mL). After cooling the reaction flask to 0° C., the addition of methyl propionylacetate (36a; R₁=ethyl; R₂=methyl) (1.41 mL, 11.2 mmol) was performed over several minutes and the resulting mixture was stirred at 30-45 min at the same temperature. To it, at the same temperature was added bromide 2 (2.85 g, 8 mmol) as 1,4-dioxane (5 mL) solution over 20 minutes. The resulting reaction mixture was then allowed to attain at room temperature and stirred for overnight (˜16 h). The reaction progress was monitored by TLC. The reaction mixture was diluted with water (˜20 mL), and was extracted with n-hexanes (3×50 mL), dried over anhydrous Na₂SO₄ and the solvent was evaporated under a reduced pressure to afford the desired keto ester 37a (R₁=ethyl; R₂=methyl), after the purification by silica gel column chromatography using n-hexanes to 1-2% EtOAc in n-hexanes. Yield: 1.79 g (65%); TLC Rf: 0.50 (10% EtOAc/n-hexanes).

### rac-3-Carboethoxy-5E,9E-farnesyl-1,1-dimethyl acetone 37b (R₁=iso-Propyl)

Similar to the preparation of ketoester 37a, the reaction of bromide 2 (8 mmol) with ethyl isobutyrylacetate (11.2 mmol) gave the desired ketoester 37b. Yield: 1.70 g (60%); TLC Rf: 0.55 (10% EtOAc/n-hexanes); LCMS: MS (m/z): 363.60 (MH+).

### rac-3-Carboethoxy-5E,9E-farnesyl-1-methyl acetone 37c (R₁=tert-Butyl)

Similar to the preparation of ketoester 37a, the reaction of bromide 2 (8 mmol) with ethyl 4,4-dimethyl-3-oxopentanoate (11.2 mmol) gave the desired ketoester 37c. Yield: 1.73 g (57%); TLC Rf: 0.33 (5% EtOAc/n-hexanes); LCMS: MS (m/z): 377.60 (MH+).

### 5E,9E-Farnesyl-1-methyl-acetone 38a (R₁=Ethyl)

A mixture of rac-ketoester 37a (1.7 g, 5 mmol), MeOH (10.0 mL), 5N aqueous KOH (5 mL) and then heated at 80-85° C. for 2 h. After cooling the reaction mixture, it was acidified with 2N HCl and extracted with diethyl ether (3×200 mL). The diethyl ether extract was successively washed with water, aqueous NaHCO₃, brine and dried over anhydrous Na₂SO₄. After removal of solvent, the oily crude product was purified by column chromatography using 1-2% EtOAc in n-hexanes to afford the desired ketone 38a. Yield: 1.00 g (72%); TLC Rf: 0.55 (10% EtOAc/n-hexanes); LCMS: MS (m/z): 277.20 (MH+).

### 5E,9E-Farnesyl-1,1-dimethyl-acetone (38b; R₁=iso-propyl)

Similar to the preparation of ketone 38a, the hydrolysis and decarboxylation of 37b (1.79 g, 4.94 mmol) afforded the desired ketone 38b. Yield: 1.3 g (90%); TLC Rf: 0.58 (10% EtOAc/n-hexanes); LCMS: MS (m/z): 291.20 (MH+).

### 5E,9E-Farnesyl-1,1,1-trimethyl-acetone (38b; R₁=tert-butyl)

Similar to the preparation of ketone 38a, the hydrolysis and decarboxylation of 37c (1.73 g, 4.6 mmol) afforded the desired ketone 38c. Yield: 1.35 g (97%); TLC Rf: 0.55 (5% EtOAc/n-hexanes); LCMS: MS (m/z): 305.20 (MH+).

### trans-Conjugated Ester 39a (R₁=ethyl)

A dry reaction flask equipped with a stir bar, N₂ inlet was charged with NaH (60% dispersed in oil; 0.202 g, 5.07 mmol) followed by a careful addition of dry THF (10 mL) and 15-crown-5 (0.02 g, catalyst). The reaction flask was cooled to 0° C. and to it was added phosphonoacetoacetate 6 (1.09 mL, 5.43 mmol) drop wise over 10-20 min. [CAUTION! Faster addition rate of phosphonoacetate can generate exotherm]. At the end of addition of phosphonoacetate, the heterogeneous reaction mixture starts turning into homogeneous or clear solution. After a complete addition, the reaction became clear solution and stir at the same temperature for 10-15 min. The clear solution was then cooled to −35 to −40° C. and to it was added ketone 38a (1.0 g, 3.62 mmol) drop wise over 10-20 min and then the resulting reaction was allowed to come to room temperature and stirred for 2-3 days. After quenching the reaction with water (50 mL) carefully, the THF layer was separated; the aqueous layer was extracted with n-hexanes (3×100 mL) and combined with THF layer. The combined organic phases were dried over Na₂SO₄ and solvent was removed under a reduced pressure to afford an oily material, from which the trans isomer 39a was separated by silica gel column chromatography using n-hexanes to 1-2% EtOAc in n-hexanes. Yield: 1.10 g (88%); TLC Rf: 0.69 (10% EtOAc/n-hexanes); LCMS: MS (m/z): 347.30 (MH+).

### Allylic Alcohol 40 (R₁=ethyl)

To a dry reaction flask was placed trans-conjugated ester 39a (1.1 g, 3.17 mmol) and THF (10 mL). At 0° C., under a N₂ atmosphere (with a vent) was added LAH (2M solution in THF, 1.58 mL, 3.17 mmol) drop wise with cautions over ˜20 min. The resulting reaction was then stirred for additional 2 h at 0° C., which was monitored by TLC. Once the reaction was completed, it was quenched with EtOAc (5 mL) followed by H₂O (5 mL) very carefully, since it generated gaseous hydrogen. The resulting jelly obtained was diluted with EtOAc (100 mL), the solid mass was filtered through celite and washed the celite pad with EtOAc (2×50 mL). The combined organic layers were dried over anhydrous Na₂SO₄ and solvent was removed under a reduced pressure, dried under high vacuum to afford 0.90 g (93%) of the desired alcohol 40. TLC Rf: 0.21 (10% EtOAc/n-hexanes). LCMS: MS (m/z): 305.40 (MH+).

### Allylic Bromide 41 (R₁=ethyl)

To a stirred solution of alcohol 40 (1.0 g, 3.28 mmol) in diethyl ether (10 mL) under N₂ at 0° C. was added phosphorous tribromide (0.101 mL, 1.09 mmol) drop wise over 5-10 min. The resulting reaction mixture was stirred at 0° C. for additional hour, which was followed by TLC. After completion of the reaction progress, it was quenched with water (2 mL), the diethyl ether was removed under a reduced pressure and the oily residue was diluted with water (30 mL). The aqueous material was then extracted with n-hexanes (3×˜30 mL), the combined hexanes were washed with brine (30 mL) dried over anhydrous MgSO₄ and concentrated under a reduced pressure to afford the desired bromide 41 which was used as such in the next step without purification to prepare the ketoesters 43.

### 3-Racemic ketoesters 43a (R₁=Ethyl; R₃=Methyl)

A reaction flask equipped with N₂ inlet, stir bar was charged with NaOEt (21% ethanolic solution, 0.210 mL, 0.65 mmol) followed by EtOH (1 mL). After cooling the reaction flask to 0° C., the addition of ethyl acetoacetate 3 (0.087 mL, 0.7 mmol) was performed dropwise and the resulting mixture was stirred at 30-45 min at the same temperature. To it, at the same temperature was added bromide 41, (0.183 g, 0.5 mmol) as 1,4-dioxane (1 mL) solution over 5 minutes. The resulting reaction mixture was then allowed to attain at room temperature and stirred for overnight (˜16 h). The reaction progress was monitored by TLC. The reaction mixture was diluted with water (˜10 mL), and was extracted with n-hexanes (3×15 mL), dried over anhydrous MgSO₄ and the solvent was evaporated under a reduced pressure to afford a crude product containing keto ester 43a and unreacted/excess ethyl acetoacetate. It was purified by silica gel column chromatography using n-hexanes to 1-2% EtOAc in n-Hexanes to afford a colorless 3-racemic keto ester 43a. Yield: 0.128 g (62%); TLC Rf: 0.42 (10% EtOAc/Hexanes), and used in the next step to prepare the corresponding ketone 44a.

### 3-Racemic ketoester 43b (R₁=Ethyl; R₂=Ethyl; R₃=iso-Propyl)

Similar to the preparation of ketoester 43a, the reaction of bromide 41 (0.183 g, 0.5 mmol) with beta-ketoester 42 (R₃=iso-propyl; R₂=ethyl) afforded the corresponding ketoester 43b. Yield: 0.130 g (59%); TLC Rf: 0.64 (7% EtOAc/n-hexanes).

### 3-Racemic ketoester 43c (R₁=Ethyl; R₂=Ethyl; R₃=1-Adamentyl)

Similar to the preparation of ketoester 43a, the reaction of bromide 41 (0.183 g, 0.5 mmol) with beta-ketoester 42 (R₃=1-adamentyl; R₂=ethyl) afforded the corresponding ketoester 43c. Yield: 0.176 g (66%); TLC Rf: 0.60 (7% EtOAc/n-hexanes).

### 3-Racemic ketoester 43d (R₁=Ethyl; R₂=Methyl; R₃=Ethyl)

Similar to the preparation of ketoester 43a, the reaction of bromide 41 (0.183 g, 0.5 mmol) with beta-ketoester 42 (R₂=methyl; R₃=ethyl) afforded the corresponding ketoester 43d. Yield: 0.149 g (72%); TLC Rf: 0.49 (10% EtOAc/n-hexanes).

### 5E,9E,13E-Geranylgeranyl acetone derivative 44a (R₁=Ethyl; R₃=Methyl)

A mixture of 3-rac-ketoester 43a (0.120 g, 0.28 mmol), MeOH (1 mL), and 5N aqueous KOH (0.5 mL) was heated at 80-85° C. for 2 h, reaction was followed by TLC. After cooling the reaction mixture, it was acidified with 2N HCl and extracted with diethyl ether, ethyl acetate or hexanes (3×10 mL). The combined organic layers were successively washed with water, aqueous NaHCO₃, brine and dried over anhydrous MgSO₄. After removal of solvent, the oily crude product was purified by silica gel column chromatography using n-hexanes to 1-2% EtOAc in n-Hexanes to afford a colorless liquid of ketone 44a. Yield: 68 mg (70%). TLC Rf: 0.53 (10% EtOAc/n-hexanes), LCMS: MS (m/z): 345.40 (MH+).

### 5E,9E,13E-Geranylgeranyl acetone derivative (44b; R₁=Ethyl; R₃=iso-Propyl)

Similar to the preparation of ketone 44a, the hydrolysis and decarboxylation of ketoester 43b (0.088 g, 0.2 mmol) afforded the corresponding ketone 44b. Yield: 0.031 g (42%), TLC Rf: 0.75 (10% EtOAc/n-hexanes); LCMS: MS (m/z): 373.40 (MH+).

### 5E,9E,13E-Geranylgeranyl acetone derivative 44c (R₁=Ethyl; R₃=1-Adamentyl)

Similar to the preparation of ketone 44a, the hydrolysis and decarboxylation of ketoester 43c (0.170 g, 0.2 mmol) afforded the corresponding ketone 44c. Yield: 0.071 g (76%), TLC Rf: 0.64 (7% EtOAc/n-hexanes); LCMS: MS (m/z): 465.60 (MH+).

### 5E,9E,13E-Geranylgeranyl acetone derivative 44a (R₁=Ethyl; R₃=Ethyl)

Similar to the preparation of ketone 44a, the hydrolysis and decarboxylation of ketoester 43d (0.066 g, 0.2 mmol) afforded the corresponding ketone 44d. Yield: 0.044 g (62%), TLC Rf: 0.54 (7% EtOAc/n-hexanes); LCMS: MS (m/z): 359.50 (MH+).

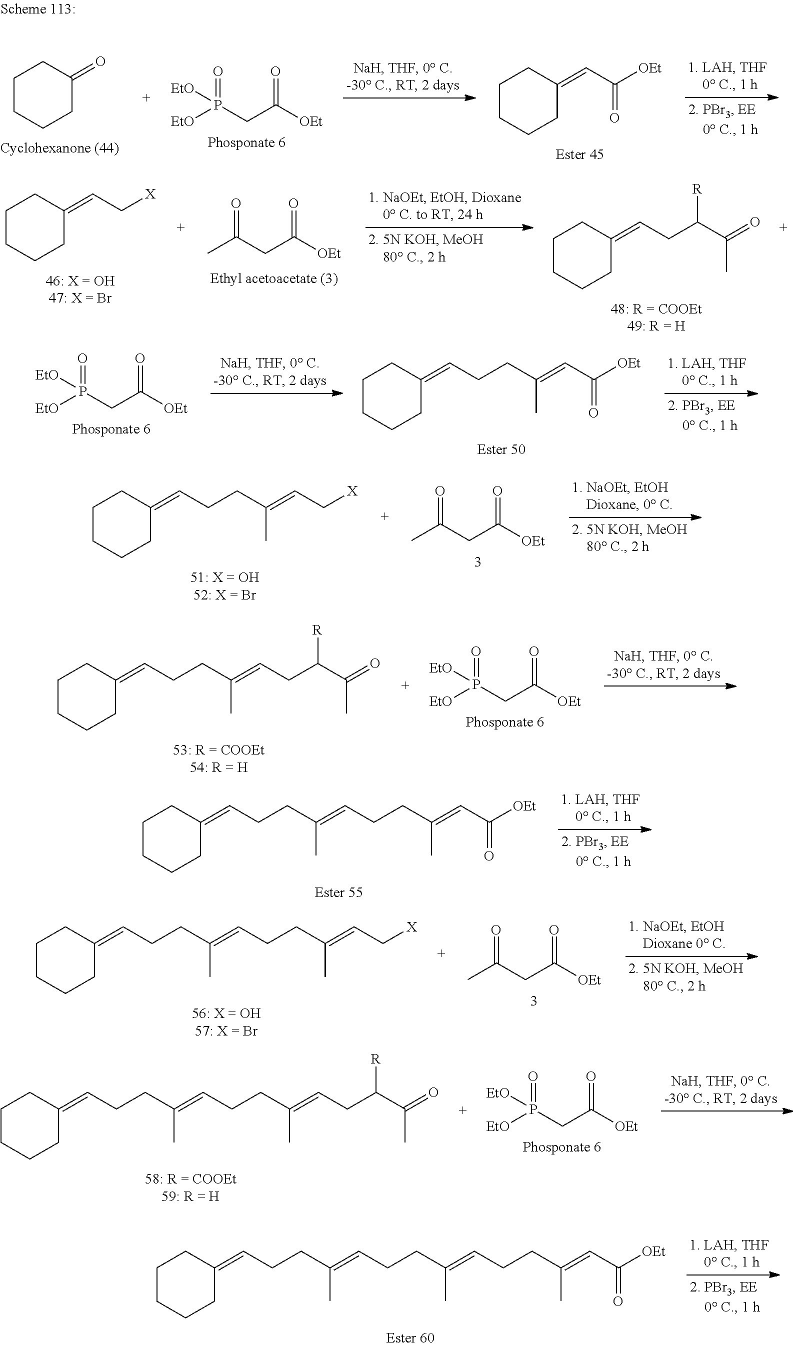

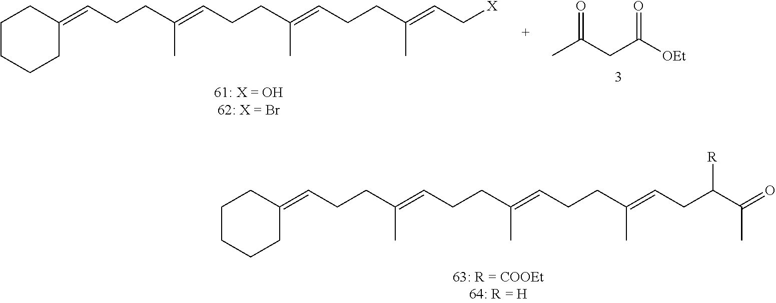

### Conjugated Ester 45

A dry reaction flask equipped with a stir bar, N₂ inlet was charged with NaH (60% dispersed in oil; 4.62 g, 130 mmol) followed by a careful addition of dry THF (200 mL) and 15-crown-5 (0.2 g, catalyst). The reaction flask was cooled to 0° C. and to it was added phosphonoacetoacetate 6 (27.75 mL, 140 mmol) drop wise over 30-45 min. [CAUTION! Faster addition rate of phosphonoacetate can generate exotherm]. At the end of addition of phosphonoacetate, the heterogeneous reaction mixture starts turning into homogeneous or clear solution. After a complete addition, the reaction became clear solution and stir at the same temperature for 10-15 min. The clear solution was then cooled to −35 to −40° C. and to it was added cyclohexanone 44 (10.34 g, 100 mmol) drop wise over ˜30 min and then the resulting reaction was allowed to come to room temperature and stirred for 2-3 days. After quenching the reaction with water (200 mL) carefully, the THF layer was separated; the aqueous layer was extracted with n-hexanes (3×200 mL) and combined with THF layer. The combined organic phases were dried over Na₂SO₄ and solvent was removed under a reduced pressure to afford an oily material, which the desired product 45 was purified by fractional distillation under a reduced pressure; 60-64° C./1 mm of Hg; Yield: 16.25 g (97%); TLC Rf: 0.15 (5% EtOAc/n-hexanes); LCMS: MS (m/z): 169.20 (MH+).

### Allylic Alcohol 46

To a dry reaction flask was placed conjugated ester 45 (13.4 g, 80 mmol) and THF (160 mL). At 0° C., under a N₂ atmosphere (with a vent) was added LAH (2M solution in THF, 40 mL, 80 mmol) drop wise with cautions over ˜40-60 min. The resulting reaction was then stirred for additional 2 h at 0° C., which was monitored by TLC. Once the reaction was completed, it was quenched with EtOAc (10 mL) followed by H₂O (20 mL) very carefully, since it generated gaseous hydrogen. The resulting jelly obtained was diluted with EtOAc (100 mL), the solid mass was filtered through celite and the celite pad was washed with EtOAc (2×50 mL). The combined organic layers were dried over anhydrous Na₂SO₄ and solvent was removed under a reduced pressure, and the resulting oily material was dried under high vacuum to afford 9.67 g (96%) of the desired alcohol 46. TLC Rf: 0.085 (10% EtOAc/n-hexanes); LCMS: MS (m/z): 109 (M− OH).

### Allylic Bromide 47

To a stirred solution of alcohol 46 (7.0 g, 55.5 mmol) in diethyl ether (70 mL) under N₂ at 0° C. was added phosphorous tribromide (1.71 mL, 18.51 mmol) drop wise over 10-15 min. The resulting reaction mixture was stirred at 0° C. for additional hour, which was followed by TLC. After completion of the reaction progress, it was quenched with water (10 mL), the diethyl ether was removed under a reduced pressure and the oily residue was diluted with water (200 mL). The aqueous material was then extracted with n-hexanes (3×˜200 mL), the combined hexanes were washed with brine (150 mL) dried over anhydrous MgSO₄ and concentrated under a reduced pressure to afford the desired bromide 47 (10.4 g, 99%) which was used as such in the next step without purification to prepare the ketoesters 48.

### Ketoester 48

A reaction flask equipped with N₂ inlet, stir bar was charged with NaOEt (21% ethanolic solution, 23.15 mL, 71.5 mmol) followed by EtOH (40 mL). After cooling the reaction flask to 0° C., the addition of ethyl acetoacetate 3 (6.94 mL, 77 mmol) was performed over several minutes and the resulting mixture was stirred at 30-45 min at the same temperature. To it, at the same temperature was added bromide 47, (10.4 g, 55 mmol) as 1,4-dioxane (40 mL) solution over 20 minutes. The resulting reaction mixture was then allowed to attain at room temperature and stirred for overnight (˜16 h). The reaction progress was monitored by TLC. The reaction mixture was diluted with water (˜50 mL), and was extracted with n-hexanes (3×200 mL), dried over anhydrous Na₂SO₄ and the solvent was evaporated under a reduced pressure to afford 8 g of a mixture of ketoester 48 and ethyl acetoacetate, which was used in the next step without purification. TLC Rf: 0.43 (10% EtOAc/n-hexanes); LCMS: MS (m/z): 237.20 (MH+).

### Ketone 49

A mixture of ketoester 48 with ethyl acetoacetate 3 (8 g), MeOH (20.0 mL), 5N aqueous KOH (10 mL) and then heated at 80-85° C. for 2 h. After cooling the reaction mixture, it was acidified with 2N HCl and extracted with diethyl ether (3×100 mL). The diethyl ether extract was successively washed with water, aqueous NaHCO₃, brine and dried over anhydrous Na₂SO₄. After removal of solvent, the oily crude product was purified by column chromatography using 1-2% EtOAc in n-hexanes to afford the desired ketone 49. Yield: 3.2 g (36%, from bromide 47); TLC Rf: 0.31 (10% EtOAc/n-hexanes); LCMS: MS (m/z): 167.10 (MH+).

### Conjugated Ester 50

A dry reaction flask equipped with a stir bar, N₂ inlet was charged with NaH (60% dispersed in oil; 1.04 g, 26 mmol) followed by a careful addition of dry THF (60 mL) and 15-crown-5 (0.02 g, catalyst). The reaction flask was cooled to 0° C. and to it was added phosphonoacetoacetate 6 (5.55 mL, 28 mmol) drop wise over 30-45 min. [CAUTION! Faster addition rate of phosphonoacetate can generate exotherm]. At the end of addition of phosphonoacetate, the heterogeneous reaction mixture starts turning into homogeneous or clear solution. After a complete addition, the reaction became clear solution and stir at the same temperature for 10-15 min. The clear solution was then cooled to −35 to −40° C. and to it was added ketone 49 (3.2 g, 20 mmol) drop wise over ˜20 min and then the resulting reaction was allowed to come to room temperature and stirred for 2-3 days. After quenching the reaction with water (20 mL) carefully, the THF layer was separated; the aqueous layer was extracted with n-hexanes (3×50 mL) and combined with THF layer. The combined organic phases were dried over Na₂SO₄ and solvent was removed under a reduced pressure to afford an oily material, which was purified by silica gel column chromatography using n-hexanes to 1-2% EtOAc in n-hexanes to afford the desired trans-conjugated ester 50 (3.2 g, 76%). TLC Rf: 0.41 (10% EtOAc/n-hexanes; LCMS: MS (m/z): 237.20 (MH+).

### Trans-Allylic Alcohol 51

To a dry reaction flask was placed trans-conjugated ester 50 (3.2 g, 13.44 mmol) and THF (50 mL). At 0° C., under a N₂ atmosphere (with a vent) was added LAH (2M solution in THF, 6.72 mL, 13.44 mmol) drop wise with cautions over ˜20 min. The resulting reaction was then stirred for additional 2 h at 0° C., which was monitored by TLC. Once the reaction was completed, it was quenched with EtOAc (5 mL) followed by H₂O (5 mL) very carefully, since it generated gaseous hydrogen. The resulting jelly obtained was diluted with EtOAc (100 mL), the solid mass was filtered through celite and the celite pad was washed with EtOAc (2×50 mL). The combined organic layers were dried over anhydrous Na₂SO₄ and solvent was removed under a reduced pressure, and the resulting oily material was dried under high vacuum to afford 2.3 g (88%) of the desired alcohol 51. TLC Rf: 0.09 (10% EtOAc/n-hexanes); LCMS: MS (m/z): 195.20 (MH+).

### Trans-Allylic Bromide 52

To a stirred solution of alcohol 51 (2.3 g, 11.85 mmol) in diethyl ether (30 mL) under N₂ at 0° C. was added phosphorous tribromide (0.366 mL, 3.95 mmol) drop wise over 10-15 min. The resulting reaction mixture was stirred at 0° C. for additional hour, which was followed by TLC. After completion of the reaction progress, it was quenched with water (5 mL), the diethyl ether was removed under a reduced pressure and the oily residue was diluted with water (50 mL). The aqueous material was then extracted with n-hexanes (3×˜75 mL), the combined hexanes were washed with brine (50 mL) dried over anhydrous MgSO₄ and concentrated under a reduced pressure to afford the desired bromide 52 (2.9 g, 99%) which was used as such in the next step without purification to prepare the ketoesters 53. TLC Rf: 0.73 (10% EtOAc/n-hexanes).

### trans-Ketoester 53

A reaction flask equipped with N₂ inlet, stir bar was charged with NaOEt (21% ethanolic solution, 5.37 mL, 16.59 mmol) followed by EtOH (7 mL). After cooling the reaction flask to 0° C., the addition of ethyl acetoacetate 3 (2.01 mL, 16.59 mmol) was performed over several minutes and the resulting mixture was stirred at 30-45 min at the same temperature. To it, at the same temperature was added bromide 52, (2.9 g, 11.7 mmol) as 1,4-dioxane (7 mL) solution over 10 minutes. The resulting reaction mixture was then allowed to attain at room temperature and stirred for overnight (˜16 h). The reaction progress was monitored by TLC. The reaction mixture was diluted with water (˜10 mL), and was extracted with n-hexanes (3×50 mL), dried over anhydrous Na₂SO₄ and the solvent was evaporated under a reduced pressure to afford a mixture of ketoester 53 and unreacted ethyl acetoacetate 3, which was used in the next step without purification. TLC Rf: 0.43 (10% EtOAc/n-hexanes).

### Trans-Ketone 54

A mixture of trans-ketoester 53 with ethyl acetoacetate 3, MeOH (20.0 mL), 5N aqueous KOH (10 mL) and then heated at 80-85° C. for 2 h. After cooling the reaction mixture, it was acidified with 2N HCl and extracted with diethyl ether (3×100 mL). The diethyl ether extract was successively washed with water, aqueous NaHCO₃, brine and dried over anhydrous Na₂SO₄. After removal of solvent, the oily crude product was purified by column chromatography using 1-2% EtOAc in n-hexanes to afford the desired ketone 54. Yield: 0.640 g (23%, from bromide 52); TLC Rf: 0.55 (10% EtOAc/n-hexanes); LCMS: MS (m/z): 235.20 (MH+).

### Trans-Conjugated Ester 55

A dry reaction flask equipped with a stir bar, N₂ inlet was charged with NaH (60% dispersed in oil; 0.141 g, 3.54 mmol) followed by a careful addition of dry THF (10 mL) and 15-crown-5 (0.01 g, catalyst). The reaction flask was cooled to 0° C. and to it was added phosphonoacetoacetate 6 (0.760 mL, 3.82 mmol) drop wise over 15 min. [CAUTION! Faster addition rate of phosphonoacetate can generate exotherm]. At the end of addition of phosphonoacetate, the heterogeneous reaction mixture starts turning into homogeneous or clear solution. After a complete addition, the reaction became clear solution and stir at the same temperature for 10-15 min. The clear solution was then cooled to −35 to −40° C. and to it was added ketone 54 (0.640 g, 2.73 mmol) drop wise over ˜20 min and then the resulting reaction was allowed to come to room temperature and stirred for 2-3 days. After quenching the reaction with water (5 mL) carefully, the THF layer was separated; the aqueous layer was extracted with n-hexanes (3×20 mL) and combined with THF layer. The combined organic phases were dried over Na₂SO₄ and solvent was removed under a reduced pressure to afford an oily material, which was purified by silica gel column chromatography using n-hexanes to 1-2% EtOAc in n-hexanes to afford the desired trans-conjugated ester 55 (0.630 g, 76%). TLC Rf: 0.52 (5% EtOAc/n-hexanes; LCMS: MS (m/z): 305.30 (MH+).

### Trans-Allylic Alcohol 56

To a dry reaction flask was placed trans-conjugated ester 55 (0.608 g, 2 mmol) and THF (10 mL). At 0° C., under a N₂ atmosphere (with a vent) was added LAH (2M solution in THF, 1.0 mL, 2 mmol) drop wise with cautions over ˜5 min. The resulting reaction was then stirred for additional 2 h at 0° C., which was monitored by TLC. Once the reaction was completed, it was quenched with EtOAc (2 mL) followed by H₂O (2 mL) very carefully, since it generated gaseous hydrogen. The resulting jelly obtained was diluted with EtOAc (25 mL), the solid mass was filtered through celite and the celite pad was washed with EtOAc (2×20 mL). The combined organic layers were dried over anhydrous Na₂SO₄ and solvent was removed under a reduced pressure, and the resulting oily material was dried under high vacuum to afford 0.500 g (95%) of the desired alcohol 56. TLC Rf: 0.10 (10% EtOAc/n-hexanes), used in the next step based on TLC characterization.

### Trans-Allylic Bromide 57

To a stirred solution of alcohol 56 (0.5 g, 1.90 mmol) in diethyl ether (5 mL) under N₂ at 0° C. was added phosphorous tribromide (0.063 mL, 3.95 mmol) drop wise. The resulting reaction mixture was stirred at 0° C. for additional hour, which was followed by TLC. After completion of the reaction progress, it was quenched with water (2 mL), the diethyl ether was removed under a reduced pressure and the oily residue was diluted with water (10 mL). The aqueous material was then extracted with n-hexanes (3×˜20 mL), the combined hexanes were washed with brine (50 mL) dried over anhydrous MgSO₄ and concentrated under a reduced pressure to afford the desired bromide 57, which was used as such in the next step without purification to prepare the ketoesters 58.

### trans-Ketoester 58

A reaction flask equipped with N₂ inlet, stir bar was charged with NaOEt (21% ethanolic solution, 0.799 mL, 2.47 mmol) followed by EtOH (1 mL). After cooling the reaction flask to 0° C., the addition of ethyl acetoacetate 3 (0.366 mL, 2.66 mmol) was performed over several minutes and the resulting mixture was stirred at 30-45 min at the same temperature. To it, at the same temperature was added bromide 57, as 1,4-dioxane (1 mL) solution dropwise. The resulting reaction mixture was then allowed to attain at room temperature and stirred for overnight (˜16 h). The reaction progress was monitored by TLC. The reaction mixture was diluted with water (˜5 mL), and was extracted with n-hexanes (3×20 mL), dried over anhydrous Na₂SO₄ and the solvent was evaporated under a reduced pressure to afford a mixture of ketoester 58 and unreacted ethyl acetoacetate 3, which was used in the next step without purification. TLC Rf: 0.36 (10% EtOAc/n-hexanes).

### Trans-Ketone 59

A mixture of trans-ketoester 58 with ethyl acetoacetate 3, MeOH (2.0 mL), 5N aqueous KOH (1.0 mL) and then heated at 80-85° C. for 2 h. After cooling the reaction mixture, it was acidified with 2N HCl and extracted with diethyl ether (3×10 mL). The diethyl ether extract was successively washed with water, aqueous NaHCO₃, brine and dried over anhydrous Na₂SO₄. After removal of solvent, the oily crude product was purified by column chromatography using 1-2% EtOAc in n-hexanes to afford the desired ketone 59. Yield: 0.180 g (31%, from bromide 57); TLC Rf: 0.42 (10% EtOAc/n-hexanes); LCMS: MS (m/z): 303.30 (MH+).

### Trans-Conjugated Ester 60

A dry reaction flask equipped with a stir bar, N₂ inlet was charged with NaH (60% dispersed in oil; 0.029 g, 0.73 mmol) followed by a careful addition of dry THF (2 mL) and 15-crown-5 (0.005 g, catalyst). The reaction flask was cooled to 0° C. and to it was added phosphonoacetoacetate 6 (0.155 mL, 0.784 mmol) drop wise. [CAUTION! Faster addition rate of phosphonoacetate can generate exotherm]. At the end of addition of phosphonoacetate, the heterogeneous reaction mixture starts turning into homogeneous or clear solution. After a complete addition, the reaction became clear solution and stir at the same temperature for 10-15 min. The clear solution was then cooled to −35 to −40° C. and to it was added ketone 59 (0.170 g, 0.56 mmol) drop wise and then the resulting reaction was allowed to come to room temperature and stirred for 2-3 days. After quenching the reaction with water (5 mL) carefully, the THF layer was separated; the aqueous layer was extracted with n-hexanes (3×10 mL) and combined with THF layer. The combined organic phases were dried over Na₂SO₄ and solvent was removed under a reduced pressure to afford an oily material, which was purified by silica gel column chromatography using n-hexanes to 1-2% EtOAc in n-hexanes to afford the desired trans-conjugated ester 60 (0.170 g, 82%). TLC Rf: 0.67 (5% EtOAc/n-hexanes; LCMS: MS (m/z): 373.30 (MH+).

### Trans-Allylic Alcohol 61

To a dry reaction flask was placed trans-conjugated ester 60 (0.170 g, 0.456 mmol) and THF (5 mL). At 0° C., under a N₂ atmosphere (with a vent) was added LAH (2M solution in THF, 0.228 mL, 0.456 mmol) drop wise with cautions over ˜5 min. The resulting reaction was then stirred for additional 2 h at 0° C., which was monitored by TLC. Once the reaction was completed, it was quenched with EtOAc (1 mL) followed by H₂O (1 mL) very carefully, since it generated gaseous hydrogen. The resulting jelly obtained was diluted with EtOAc (10 mL), the solid mass was filtered through celite and the celite pad was washed with EtOAc (2×10 mL). The combined organic layers were dried over anhydrous Na₂SO₄ and solvent was removed under a reduced pressure, and the resulting oily material was dried under high vacuum to afford 0.130 g (86%) of the desired alcohol 61. TLC Rf: 0.10 (10% EtOAc/n-hexanes).

### Trans-Allylic Bromide 62

To a stirred solution of alcohol 61 (0.130 g, 0.393 mmol) in diethyl ether (5 mL) under N₂ at 0° C. was added phosphorous tribromide (0.012 mL, 0.131 mmol) drop wise. The resulting reaction mixture was stirred at 0° C. for additional hour, which was followed by TLC. After completion of the reaction progress, it was quenched with water (2 mL), the diethyl ether was removed under a reduced pressure and the oily residue was diluted with water (10 mL). The aqueous material was then extracted with n-hexanes (3×˜10 mL), the combined hexanes were washed with brine (10 mL) dried over anhydrous MgSO₄ and concentrated under a reduced pressure to afford the desired bromide 62, which was used as such in the next step without purification to prepare the ketoesters 63.

### trans-Ketoester 63

A reaction flask equipped with N₂ inlet, stir bar was charged with NaOEt (21% ethanolic solution, 0.164 mL, 0.507 mmol) followed by EtOH (0.5 mL). After cooling the reaction flask to 0° C., the addition of ethyl acetoacetate 3 (0.069 mL, 0.546 mmol) was performed over several minutes and the resulting mixture was stirred at 30-45 min at the same temperature. To it, at the same temperature was added bromide 62, as 1,4-dioxane (0.5 mL) solution dropwise. The resulting reaction mixture was then allowed to attain at room temperature and stirred for overnight (˜16 h). The reaction progress was monitored by TLC. The reaction mixture was diluted with water (˜5 mL), and was extracted with n-hexanes (3×10 mL), dried over anhydrous Na₂SO₄ and the solvent was evaporated under a reduced pressure to afford a mixture of ketoester 63 and unreacted ethyl acetoacetate 3, which was used in the next step without purification. TLC Rf: 0.38 (5% EtOAc/n-hexanes).

### Trans-Ketone 64

A mixture of trans-ketoester 63 with ethyl acetoacetate 3, MeOH (2.0 mL), 5N aqueous KOH (1.0 mL) and then heated at 80-85° C. for 2 h. After cooling the reaction mixture, it was acidified with 2N HCl and extracted with diethyl ether (3×10 mL). The diethyl ether extract was successively washed with water, aqueous NaHCO₃, brine and dried over anhydrous Na₂SO₄. After removal of solvent, the oily crude product was purified by column chromatography using 1-2% EtOAc in n-hexanes to afford the desired ketone 64. Yield: 0.028 g (17%, from bromide 57); TLC Rf: 0.41 (5% EtOAc/n-hexanes); LCMS: MS (m/z): 371.40 (MH+).

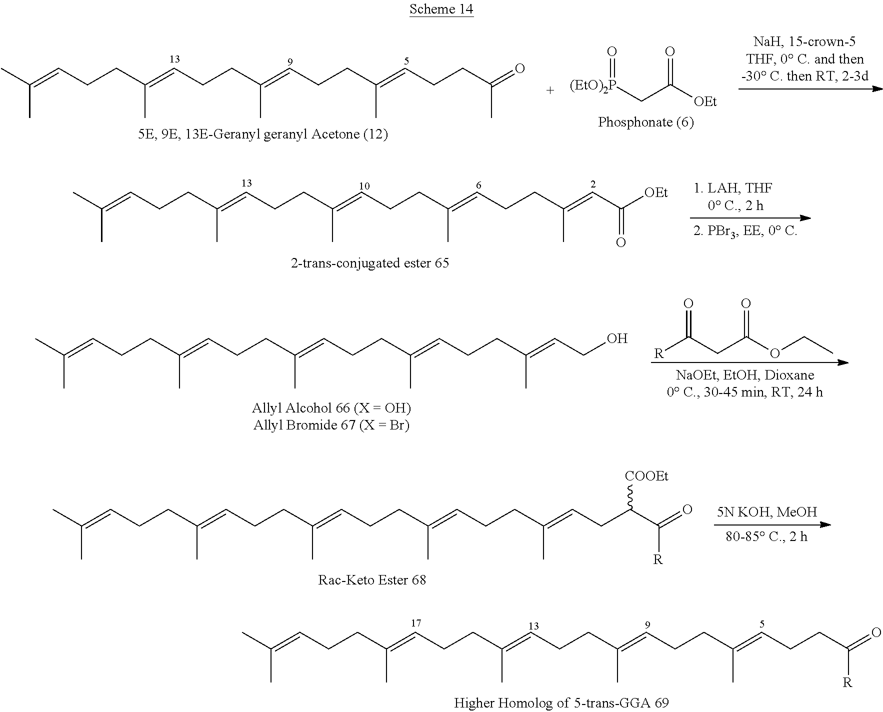

### trans-2E,6E,10E,13E-Conjugated Ester 65

A dry reaction flask equipped with a magnetic stirring bar, N₂ inlet and rubber septum was charged with NaH (60% disp. in oil; 0.278 g, 7 mmol), 15-crown-5 (0.05 mL) and anhydrous THF (10 mL). The resulting suspension was cooled 0° C. and to it was added triethyl phoponoacetoacetate 6 (1.51 mL, 7.5 mmol) carefully and dropwise. As the addition of 6 was in progress the heterogeneous material was turning clear and became completely clear after the addition was completed. The resulting clear solution was stirred for another 15 minutes and then was cooled to −30° C. To it was added the ketone 12 (1.66 g, 5 mmol) as a THF (10 mL) solution over a period of 15-20 minutes. The resulting mixture was allowed to warm to the room temperature and then stirred at RT for 2 days. After quenching the reaction with water (25 mL) carefully, the THF layer was separated; the aqueous layer was extracted with n-hexanes (3×50 mL) and combined with THF layer. The combined organic phases were dried over Na₂SO₄ and solvent was removed under a reduced pressure to afford an oily material, from which the trans-isomer 65 was isolated by silica gel column chromatography using n-hexanes to 1% EtOAc in hexanes. Yield: 1.7 g, (85%) TLC Rf: 0.39 (5% EtOAc/n-hexanes); LCMS: MS (m/e) 401.60 (MH+).

### trans-Allylic Alcohol 66

To a dry reaction flask was placed trans-conjugated ester 65 (1.7 g, 4.25 mmol) and THF (10 mL). At 0° C., under a N₂ atmosphere (with a vent) was added LAH (2M solution in THF, 2.12 mL, 4.25 mmol) drop wise with cautions over 20 min. The resulting reaction was then stirred for additional 1 h at 0° C., which was monitored by TLC. Once the reaction was completed, it was quenched with EtOAc (3 mL) followed by H₂O (3 mL) very carefully, since it generated gaseous hydrogen. The resulting jelly obtained was diluted with EtOAc (50 mL), the solid mass was filtered through celite and washed the celite pad with EtOAc (2×50 mL). The combined organic layers were dried over anhydrous Na₂SO₄ and solvent was removed under a reduced pressure, dried under high vacuum to afford 1.35 g (91%) of the desired alcohol 66. TLC Rf: 0.14 (10% EtOAc/n-hexanes); LCMS: MS (m/z): 251.6 (MH+).

### trans-Allylic Bromide 67

To a stirred solution of alcohol 66 (0.800 g, 2.23 mmol) in diethyl ether (10 mL) under N2 at 0° C. was added phosphorous tribromide (0.070 mL, 0.744 mmol) drop wise over 10 min. The resulting reaction mixture was stirred at 0° C. for additional hour, which was followed by TLC. After completion of the reaction progress, it was quenched with water (5 mL), the diethyl ether was removed under a reduced pressure and the oily residue was diluted with water (20 mL). The aqueous material was then extracted with n-hexanes (3×20 mL), the combined hexanes were washed with brine (50 mL) dried over anhydrous MgSO₄ and concentrated under a reduced pressure to afford the desired trans-allylic bromide 67 (crude, 0.826 g, ˜89%). The bromide was dried under high vacuum and used in the next step without any additional purification to prepare ketoesters 68.

### 3-Racemic ketoester 68a (R=Methyl)

A reaction flask equipped with N₂ inlet, stir bar was charged with NaOEt (21% ethanolic solution, 0.463 mL, 1.43 mmol) followed by EtOH (1 mL). After cooling the reaction flask to 0° C., the addition of ethyl acetoacetate 3 (0.194 mL, 1.54 mmol) was performed over several minutes and the resulting mixture was stirred at 30-45 min at the same temperature. To it at the same temperature was added bromide 67 (0.413 g, 0.98 mmol) as 1,4-dioxane (1 mL) solution over 10-15 minutes. The resulting reaction mixture was then allowed to attain at room temperature and stirred for overnight (˜16 h). The reaction progress was monitored by TLC. The reaction mixture was diluted with water (˜10 mL), and was extracted with n-hexanes (3×15 mL), dried over anhydrous Na₂SO₄ and the solvent was evaporated under a reduced pressure to afford keto ester 68a after the purification by silica gel column chromatography using 1-2% EtOAc/n-hexanes. TLC Rf: 0.44 (5% EtOAc/n-hexanes); LCMS: MS (m/z): 472 (MH+).

### Ketone 69a (R=Methyl)

A mixture of resulting 3-rac-ketoester 68a, MeOH (3 mL), and 5N aqueous KOH (1.5 mL) was heated at 80-85° C. for 2 h, reaction was followed by TLC. After cooling the reaction mixture, it was acidified with 2N HCl and extracted with diethyl ether, ethyl acetate or hexanes (3×20 mL). The combined organic layers were successively washed with water, aqueous NaHCO₃, brine and dried over anhydrous MgSO₄. After removal of solvent, the oily crude product was purified by silica gel column chromatography using n-hexanes to 1-2% EtOAc in n-Hexanes to afford a colorless liquid of ketone 68a. Yield: 0.198 g (51%). TLC Rf: 0.55 (5% EtOAc/n-hexanes); LCMS: MS (m/z): 399.40 (MH+).

### 3-Racemic ketoester 68b (R=Cyclopropyl)

A reaction flask equipped with N₂ inlet, stir bar was charged with NaOEt (21% ethanolic solution, 0.463 mL, 1.43 mmol) followed by EtOH (1 mL). After cooling the reaction flask to 0° C., the addition of ethyl 3-cyclopropyl-3-oxopropanoate (0.227 mL, 1.54 mmol) was performed over several minutes and the resulting mixture was stirred at 30-45 min at the same temperature. To it at the same temperature was added bromide 67 (0.413 g, 0.98 mmol) as 1,4-dioxane (1 mL) solution over 10-15 minutes. The resulting reaction mixture was then allowed to attain at room temperature and stirred for overnight (˜16 h). The reaction progress was monitored by TLC. The reaction mixture was diluted with water (˜10 mL), and was extracted with n-hexanes (3×15 mL), dried over anhydrous Na₂SO₄ and the solvent was evaporated under a reduced pressure to afford a crude product containing keto ester 68b and unreacted/excess ethyl 3-cyclopropyl-3-oxopropanoate. The crude material was then used to hydrolyze and decarboxylate to give ketone 69b, without any purification, TLC Rf: 0.52 (5% EtOAc/n-hexanes).

### Ketone 69b (R=Cyclopropyl)

A mixture of resulting crude 3-rac-ketoester 68b, MeOH (3 mL), and 5N aqueous KOH (1.5 mL) was heated at 80-85° C. for 2 h, reaction was followed by TLC. After cooling the reaction mixture, it was acidified with 2N HCl and extracted with diethyl ether, ethyl acetate or hexanes (3×20 mL). The combined organic layers were successively washed with water, aqueous NaHCO₃, brine and dried over anhydrous MgSO₄. After removal of solvent, the oily crude product was purified by silica gel column chromatography using n-hexanes to 1-2% EtOAc in n-Hexanes to afford a colorless liquid of ketone 68b. Yield: 0.199 g (48%). TLC Rf: 0.66 (5% EtOAc/n-hexanes); LCMS: MS (m/z): 425.40 (MH+).

### Example 2

### Compounds Provide In Vitro Neuroprotection

Neuro2A cells were cultured with a GGA derivative in the presence or absence of an inhibitor against a G-protein (GGTI-298). After differentiation was induced, cells that extended neurites were counted as an activity of the compound. The activities of the compounds at 1 nM, 10 nM, and 104 were calculated for certain analogs and are tabulated in table 1. The activities are shown in arbitrary units and were normalized by the activity of CNS-102 (GGA trans-isomer) in each parallel experiments. For each of the compounds listed in table 1, the activity data provided showed an increase in activity over a control experiment with no addition of compound, unless otherwise indicated.

### Example 3

### Efficacy of Compounds in Alleviating Neurodegeneration Induced by Kainic Acid

The indicated compounds or vehicle control were orally dosed to Sprague-Dawley rats, and Kainic acid was injected. Seizure behaviors were observed and scored (Ref. R. J. Racine, Modification of seizure activity by electrical stimulation: 11. Motor seizure, Electroencephalogr. Clin. Neurophysiol. 32 (1972) 281-294. Modifications were made for the methods). Brain tissues of rats were sectioned on histology slides, and neurons in hippocampus tissues were stained by Nissl. Neurons damaged by Kainic acid (mean of hippocampus CA3 neurons damaged) and behavior scores (mean of seizure behavior scores) were quantified. These results are depicted in the following tables:

| | Mean of Hippocampus |
|---|---|
| | CA3 neurons damaged |
| Compounds | (Arbitrary units) |
| 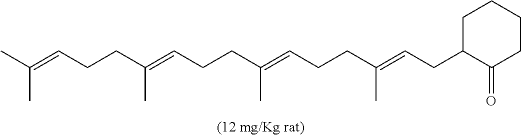 [SMILES: CC(C)=CCC/C(C)=C/CC/C(C)=C/CC/C(C)=C/CC1CCCCC1=O] | 2.64 |
| 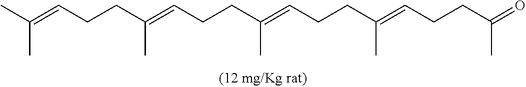 [SMILES: CC(=O)CC/C=C(\C)CC/C=C(\C)CC/C=C(\C)CCC=C(C)C] | 9.46 |
| Vehicle | 15.61 |

| | Mean of Hippocampus |
|---|---|
| | CA3 neurons damaged |
| Compounds | (Arbitrary units) |
| 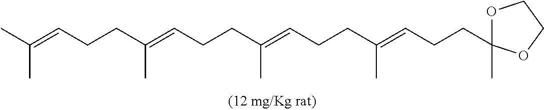 [SMILES: CC(C)=CCC/C(C)=C/CC/C(C)=C/CC/C(C)=C/CCC1(C)OCCO1] | 12.11 |
| Vehicle | 15.31 |

| | Mean of Hippocampus |
|---|---|
| | CA3 neurons damaged |
| Compounds | (Arbitrary units) |
| 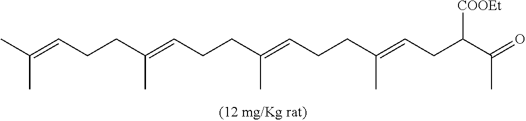 [SMILES: CCOC(=O)C(C/C=C(\C)CC/C=C(\C)CC/C=C(\C)CCC=C(C)C)C(C)=O] | ~5.57 |
| Vehicle | ~7.74 |

| | Mean of Hippocampus |
|---|---|
| | CA3 neurons damaged |
| Compounds | (Arbitrary units) |
| 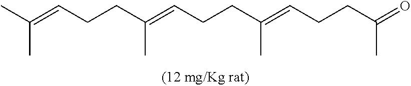 [SMILES: CC(=O)CC/C=C(\C)CC/C=C(\C)CCC=C(C)C] | 10.61 |
| 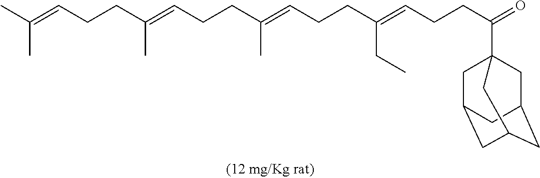 [SMILES: CC/C(=C\CCC(=O)C12CC3CC(CC(C3)C1)C2)CC/C=C(\C)CC/C=C(\C)CCC=C(C)C] | 12.54 |
| Vehicle | 19.95 |

| | Mean of Hippocampus |
|---|---|
| | CA3 neurons damaged |
| Compounds | (Arbitrary units) |
| 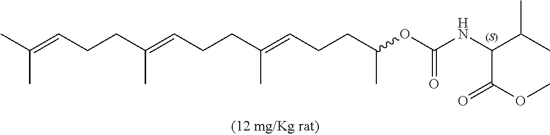 | 6.61 |
| Vehicle | 9.76 |

| | Mean of Hippocampus |
|---|---|
| | CA3 neurons damaged |
| Compounds | (Arbitrary units) |
| 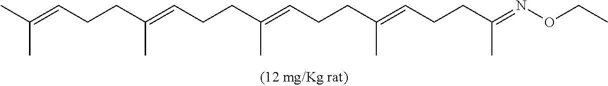 [SMILES: CCO/N=C(\C)CC/C=C(\C)CC/C=C(\C)CC/C=C(\C)CCC=C(C)C] | 14.66 |
| Vehicle | 17.59 |

| | Mean of Hippocampus |
|---|---|
| | CA3 neurons damaged |
| Compounds | (Arbitrary units) |
| 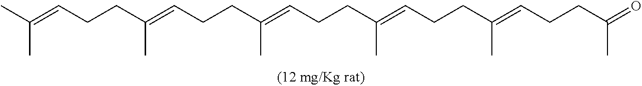 [SMILES: CC(=O)CC/C=C(\C)CC/C=C(\C)CC/C=C(\C)CC/C=C(\C)CCC=C(C)C] | 2.22 |
| Vehicle | 12.71 |

| | Mean of Hippocampus |
|---|---|
| | CA3 neurons damaged |
| Compounds | (Arbitrary units) |
| 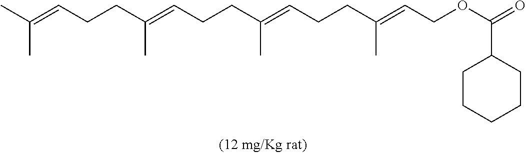 [SMILES: CC(C)=CCC/C(C)=C/CC/C(C)=C/CC/C(C)=C/COC(=O)C1CCCCC1] | 1.55 |
| 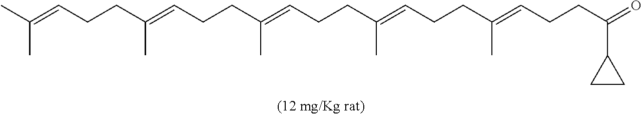 [SMILES: CC(C)=CCC/C(C)=C/CC/C(C)=C/CC/C(C)=C/CC/C(C)=C/CCC(=O)C1CC1] | 5.98 |
| Vehicle | 6.59 |

| | Mean of Hippocampus |
|---|---|
| | CA3 neurons damaged |
| Compounds | (Arbitrary units) |
| 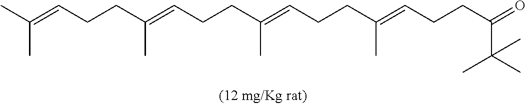 [SMILES: CC(C)=CCC/C(C)=C/CC/C(C)=C/CC/C(C)=C/CCC(=O)C(C)(C)C] | 8.08 |
| Vehicle | 12.91 |

| | Mean of Hippocampus |
|---|---|
| | CA3 neurons damaged |
| Compounds | (Arbitrary units) |
| 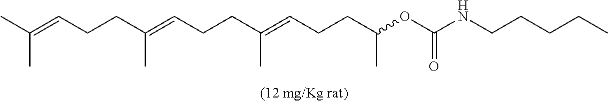 [SMILES: CCCCCNC(=O)OC(C)CC/C=C(\C)CC/C=C(\C)CCC=C(C)C] | 14.73 |
| Vehicle | 7.09 |

| | Mean of Hippocampus |
|---|---|
| | CA3 neurons damaged |
| Compounds | (Arbitrary units) |
| 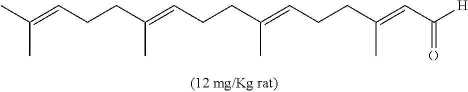 [SMILES: CC(C)=CCC/C(C)=C/CC/C(C)=C/CC/C(C)=C/C=O] | 1.58 |
| Vehicle | 1.72 |

| | Mean of Seizure |
|---|---|
| | behaviors scores |
| Compounds | (Arbitrary units) |
| 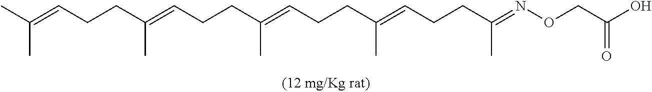 [SMILES: CC(C)=CCC/C(C)=C/CC/C(C)=C/CC/C(C)=C/CC/C(C)=N/OCC(=O)O] | 13.37 |
| Vehicle | 26.68 |

| | Mean of Seizure |
|---|---|
| | behaviors scores |
| Compounds | (Arbitrary units) |
| 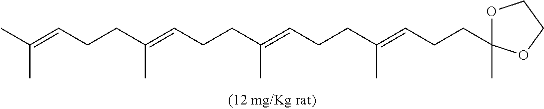 [SMILES: CC(C)=CCC/C(C)=C/CC/C(C)=C/CC/C(C)=C/CCC1(C)OCCO1] | 16.35 |
| Vehicle | 41.56 |

| | Mean of Seizure |
|---|---|
| | behaviors scores |
| Compounds | (Arbitrary units) |
| 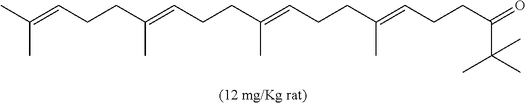 [SMILES: CC(C)=CCC/C(C)=C/CC/C(C)=C/CC/C(C)=C/CCC(=O)C(C)(C)C] | 11.25 |
| Vehicle | 19.75 |

| | Mean of Seizure |
|---|---|
| | behaviors scores |
| Compounds | (Arbitrary units) |
| 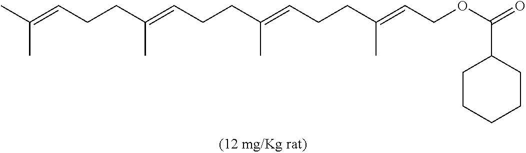 [SMILES: CC(C)=CCC/C(C)=C/CC/C(C)=C/CC/C(C)=C/COC(=O)C1CCCCC1] | 18.68 |
| Vehicle | 26.31 |

| | Mean of Seizure |
|---|---|
| | behaviors scores |
| Compounds | (Arbitrary units) |
| 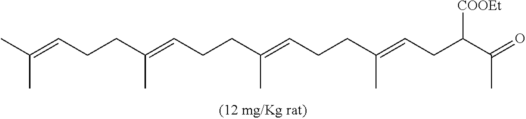 [SMILES: CCOC(=O)C(C/C=C(\C)CC/C=C(\C)CC/C=C(\C)CCC=C(C)C)C(C)=O] | 3.83 |
| Vehicle | 13.37 |

These results indicate that the compounds provide protection to neurons from neuronal damage. It is contemplated that such effects of trans-GGA also renders it useful for protecting tissue damage during seizures, ischemic attacks, and neural impairment such as in glaucoma.

### Example 4

### Expression of Heat Shock Proteins In Vitro

Mouse Neuro2A neuroblastoma cells were cultured in DMEM supplemented with 10% FBS for 24 hrs. The cells were treated with various concentrations of the indicated compounds. Then differentiation was induced by retinoic acid in DMEM supplemented with 2% FBS. An inhibitor against a G-protein, GGTI-298, was incubated. After 24 hrs incubation, cells were harvested, and lysates were prepared from the harvested cells. Western blotting analysis was done for the same protein amounts of these lysates, and western signals were detected by chemiluminescence and quantified in parallel with comparisons of those detected in the absence of the compound. Western signals in the absence of the compounds were normalized as 1. These results are depicted in the table below:

| | HSP70 | | |
|---|---|---|---|
| Compound | 100 | | |
| Dose | nM | 1 μM | 10 μM |
| 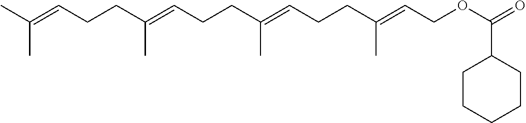 [SMILES: CC(C)=CCC/C(C)=C/CC/C(C)=C/CC/C(C)=C/COC(=O)C1CCCCC1] | 2.53 | 2.34 | 2.53 |
| 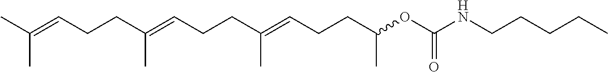 [SMILES: CCCCCNC(=O)OC(C)CC/C=C(\C)CC/C=C(\C)CCC=C(C)C] | 1.16 | 1.26 | 1.93 |
| 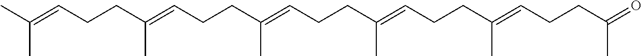 [SMILES: CC(=O)CC/C=C(\C)CC/C=C(\C)CC/C=C(\C)CC/C=C(\C)CCC=C(C)C] | 1.92 | 2.78 | 3.53 |
| 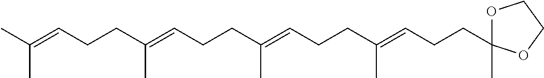 [SMILES: CC(C)=CCC/C(C)=C/CC/C(C)=C/CC/C(C)=C/CCC1(C)OCCO1] | 3.16 | 3.50 | 3.49 |
| 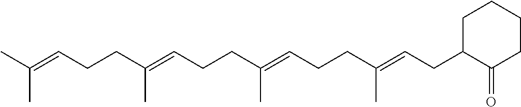 [SMILES: CC(C)=CCC/C(C)=C/CC/C(C)=C/CC/C(C)=C/CC1CCCCC1=O] | 3.85 | 3.78 | 2.25 |
| 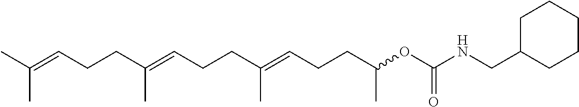 [SMILES: CC(C)=CCC/C(C)=C/CC/C(C)=C/CCC(C)OC(=O)NCC1CCCCC1] | 1.55 | 1.49 | 2.53 |
| 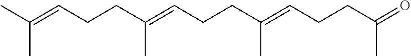 [SMILES: CC(=O)CC/C=C(\C)CC/C=C(\C)CCC=C(C)C] | 1.87 | 5.86 | 1.79 |
| 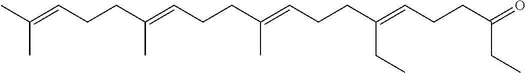 [SMILES: CCC(=O)CC/C=C(\CC)CC/C=C(\C)CC/C=C(\C)CCC=C(C)C] | 1.79 | 6.08 | 4.76 |
| 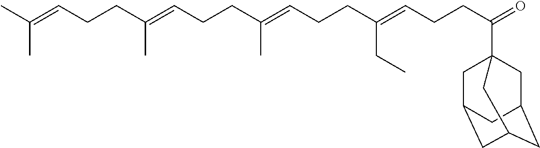 [SMILES: CC/C(=C\CCC(=O)C12CC3CC(CC(C3)C1)C2)CC/C=C(\C)CC/C=C(\C)CCC=C(C)C] | 1.71 | 2.00 | 1.00 |
| 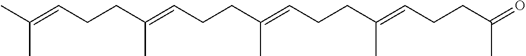 [SMILES: CC(=O)CC/C=C(\C)CC/C=C(\C)CC/C=C(\C)CCC=C(C)C] | 1.38 | 2.05 | 2.14 |
| 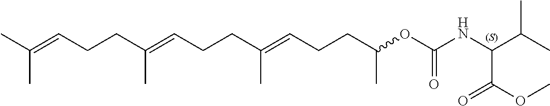 | 1.26 | 1.07 | 1.29 |

### Example 4

### Expression of Heat Shock Proteins In Vivo

GGA trans isomer in 5% Gum Arabic as an aqueous suspension formulation were orally dosed to Sprague-Dawley rats at 12 mg/Kg body weight. Rat brain tissues were extracted in various time points after the oral dosing. mRNA were prepared from those brain tissues extracted, and cDNA were produced. qPCR analysis was done by using primers specifically designed to detect mRNA of HSPs. GAPDH gene was used as a control to compare quantities of HSP cDNAs amplified by qPCR analysis. Amounts of cDNA quantified at time 0 are normalized as 100%, and relative amounts of cDNA compared with those at various time points are depicted in the tables below:

| Time | HSP27 | HSP90 | HSP70 | HSP60 | GAPDH |
|---|---|---|---|---|---|
| 0 hr | 100% | 100% | 100% | 100% | 100% |
| 12 hr | 115.38% | 107.69% | 118.46% | 113.84% | 96.93% |
| 24 hr | 114.61% | 106.92% | 107.69% | 130.00% | 99.24% |
| 48 hr | 116.15% | 105.38% | 106.15% | 116.92% | 100% |
| 96 hr | 103.84% | 100.77% | 103.85% | 103.85% | 102.31% |

The compounds tabulated below or vehicle controls were orally dosed at 12 mg/kg to Sprague-Dawley rats. Rat brain tissues were extracted in various time points after the oral dosing. Lysates were prepared from the harvested brain tissues. Western blotting analysis was done for the same protein amounts of these lysates, and western signals of HSP70 were detected by chemiluminescence and quantified in parallel with comparisons of those detected at time 0 hr. Western signals at time 0 hr were normalized as 100. These results are depicted in the table below.

| Time | Vehicle | 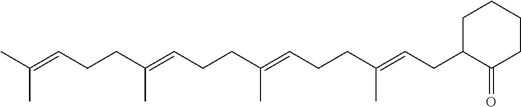 | Trans GGA |
|---|---|---|---|
| 0 hr | 100% | 100% | 100% |
| 12 hr | 102% | 116% | 115% |
| 48 hr | 105% | 114% | 115% |
| 96 hr | 99% | 97% | 114% |

1. A compound of formula: 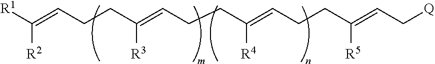
wherein
m is 0 or 1;
n is 0, 1, or 2;
each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups;
each of R³, R⁴, and R⁵ independently are hydrogen or C₁-C₆ alkyl;
Q is selected from the group consisting of: 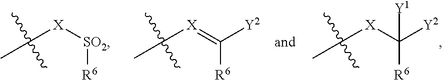
when X is bonded via a single bond, X is —O—, —NR⁷—, or —CR⁸R⁹—, and when X is bonded via a double bond, X is —CR⁸—;
Y¹ is hydrogen or —O—R¹⁰, Y² is —OR¹¹ or —NHR¹², or Y¹ and Y² are joined to form an oxo group (═O), an imine group (═NR¹³), a oxime group (═N—OR¹⁴), or a substituted or unsubstituted vinylidene (═CR¹⁶R¹⁷);
R⁶ is C₁-C₆ alkyl optionally substituted with 1-3 alkoxy or 1-5 halo group, C₂-C₆ alkenyl, C₂-C₆ alkynyl, C₃-C₁₀ cycloalkyl, C₆-C₁₀ aryl, C₃-C₈ heterocyclyl, or C₂-C₁₀ heteroaryl, wherein each cycloalkyl or heterocyclyl is optionally substituted with 1-3 C₁-C₆ alkyl groups, or wherein each aryl or heteroaryl is independently substituted with 1-3 C₁-C₆ alkyl or nitro groups;
R⁷ is hydrogen or together with R⁶ and the intervening atoms form a 5-7 membered ring optionally substituted with 1-3 C₁-C₆ alkyl groups;
each R⁸ and R⁹ independently are hydrogen, C₁-C₆ alkyl, —COR⁸¹ or —CO₂R⁸¹, or R⁸ together with R⁶ and the intervening atoms form a 5-7 membered cycloalkyl or heterocyclyl ring optionally substituted with 1-3 C₁-C₆ alkyl groups;
R¹⁰ is C₁-C₆ alkyl;
R¹¹ and R¹² are independently C₁-C₆ alkyl, C₃-C₁₀ cycloalkyl, —CO₂R¹⁵, or —CON(R¹⁵)₂, or R¹⁰ and R¹¹ together with the intervening carbon atom and oxygen atoms form a heterocycle optionally substituted with 1-3 C₁-C₆ alkyl groups;
R¹³ is C₁-C₆ alkyl or C₃-C₁₀ cycloalkyl optionally substituted with 1-3 C₁-C₆ alkyl groups;
R¹⁴ is hydrogen, C₁-C₆ alkyl optionally substituted with a —CO₂H or an ester thereof or a C₆-C₁₀ aryl, C₂-C₆ alkenyl, C₂-C₆ alkynyl, C₃-C₁₀ cycloalkyl, or a C₃-C₈ heterocyclyl, wherein each cycloalkyl, heterocyclyl, or aryl, is optionally substituted with 1-3 alkyl groups;
each R¹⁵ independently are hydrogen, C₃-C₁₀ cycloalkyl, C₁-C₆ alkyl optionally substituted with 1-3 substituents selected from the group consisting of —CO₂H or an ester thereof, C₆-C₁₀ aryl, or C₃-C₈ heterocyclyl, or two R¹⁵ groups together with the nitrogen atom they are bonded to form a 5-7 membered heterocycle;
R¹⁶ is hydrogen or C₁-C₆ alkyl;
R¹⁷ is hydrogen, C₁-C₆ alkyl substituted with 1-3 hydroxy groups, —CHO, or is CO₂H or an ester thereof; and
each R⁸¹ independently is C₁-C₆ alkyl; and
provided that the compound excludes the compound of formula: 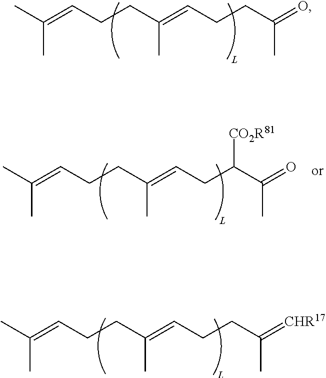
wherein L is 0, 1, 2, or 3, and R¹⁷ is CO₂H or an ester thereof or is —CH₂OH.

2-58. (canceled)

59. The compound of claim 1, wherein said compound is represented by the formula: 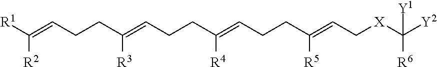
wherein R¹, R², R³, R⁴, R⁵, R⁶, X, Y¹, and Y² are defined as in claim 1.

60. The compound of claim 1, wherein said compound is represented by the formula: 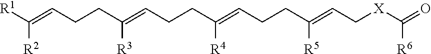
wherein R¹, R², R³, R⁴, R⁵, R⁶, and X are defined as in claim 1.

61. The compound of claim 1 of formula: 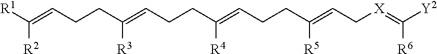
wherein R¹, R², R³, R⁴, R⁵, R⁶, X and Y² are defined as in claim 1.

62. The compound of claim 1, wherein each R¹ and R² are C₁-C₆ alkyl.

63. The compound of claim 1, wherein R¹ and R² together with the carbon atom they are attached to form a 5-6 membered ring optionally substituted with 1-3 C₁-C₆ alkyl groups.

64. The compound of claim 1, wherein each R³, R⁴, and R⁵ are C₁-C₆ alkyl.

65. The compound of claim 1, wherein X is O.

66. The compound of claim 1, wherein X is —NR⁷.

67. The compound of any one of claim 1, wherein R⁷ is hydrogen or R⁷ together with R⁶ and the intervening atoms form a 5-7 membered ring optionally substituted with 1-3 C₁-C₆ alkyl groups.

68. The compound of claim 1, wherein X is —CR⁸R⁹—.

69. The compound of claim 1, wherein each R⁸ and R⁹ independently are hydrogen, C₁-C₆ alkyl, or —CO₂R⁸¹.

70. The compound of claim 69, wherein R⁸ is hydrogen.

71. The compound of claim 70, wherein R⁹ is hydrogen or R⁹ is C₁-C₆ alkyl.

72. The compound of claim 1, wherein R⁸ together with R⁶ and the intervening atoms form a 5-7 membered ring optionally substituted with 1-3 C₁-C₆ alkyl groups.

73. The compound of claim 72, wherein R⁹ is hydrogen or C₁-C₆ alkyl.

74. The compound of claim 72, wherein R⁶ is C₁-C₆ alkyl.

75. The compound of claim 1, wherein R⁶ is C₁-C₆ alkyl substituted with an alkoxy or a halo group.

76. The compound of claim 1, wherein R⁶ is C₂-C₆ alkenyl, or C₂-C₆ alkynyl.

77. The compound of claim 1, wherein R⁶ is C₃-C₁₀ cycloalkyl.

78. The compound of claim 1, wherein R⁶ is C₆-C₁₀ aryl or C₂-C₁₀ heteroaryl.

79. The compound of claims 1, wherein the moiety: 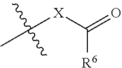
has the structure: 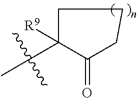
wherein R⁹ is hydrogen, alkyl, or —CO₂R⁸¹ and n is 1, 2, or 3.

80. The compound of claim 1, wherein Y² is —O—R¹¹.

81. The compound of claim 1, wherein Y¹ and Y² are joined to form (═NR¹³).

82. The compound of claim 1, wherein Y¹ and Y² are joined to form (═O).

83. The compound of any one of claim 1, wherein m is 1 and n is 1.

84. A composition comprising a compound of claim 1 and a pharmaceutically acceptable excipient.

85. A method for treating a neuron in need thereof of one or more of:
(i) neuroprotection of the neuron at risk of neural damage or death,
(ii) increasing the axon growth of the neuron,
(iii) inhibiting the cell death of the neuron susceptible to neuronal cell death,
(iv) increasing the neurite growth of the neuron, and/or
(v) neurostimulation comprising increasing the expression and/or the release of one or more neurotransmitters from the neuron,
the method comprising contacting said neurons with an effective amount of a compound of claim 1.

86. A method for treating a neuron in need thereof of one or more of:
(i) neuroprotection of the neuron at risk of neural damage or death,
(ii) increasing the axon growth of the neuron,
(iii) inhibiting the cell death of the neuron susceptible to neuronal cell death,
(iv) increasing the neurite growth of the neuron, and/or
(v) neurostimulation comprising increasing the expression and/or the release of one or more neurotransmitters from the neuron,
the method comprising contacting said neurons with an effective amount of a composition of claim 84.
